(19) United States
(12) Patent Application Publication
(10) Pub. No.: US 2005/0255537 A1
(43) Pub. Date: Nov. 17, 2005

(54) Method for monitoring treatment with a parathyroid hormone

(76) Inventors: Janet Mary Hock, Indianapolis, IN (US); Julie H. Satterwhite, Zionsille, IN (US)

Correspondence Address: ELI LILLY AND COMPANY;PATENT DIVISION, P.O. BOX 6288, INDIANAPOLIS, IN, 46206-6288, US

(21) Appl. No.: 11/151,907

(22) Filed: Jun. 14, 2005

Related U.S. Application Data
(63) Continuation of application No. 10/070,660, filed on Aug. 27, 2002, now abandoned.
(60) Provisional application No. 60/154,879, filed on Sep. 20, 1999.
(60) Provisional application No. 60/156,803, filed on Sep. 30, 1999.
(60) Provisional application No. 60/196,370, filed on Apr. 12, 2000.

(51) Int. Cl. A61K 31/198; C12Q 1/42

(52) U.S. Cl. 435/21; 514/567

(57) ABSTRACT

The present invention relates to a method for monitoring effects of administration of a parathyroid hormone by determining levels of one or more markers of an activity of this hormone. Suitable markers of bone formation include one or more enzymes indicative of osteoblastic processes of bone formation, preferably bone specific alkaline phosphatase, and/or one or more products of collagen biosynthesis, preferably a procollagen I C-terminal propetide. Suitable markers of bone resorption and turnover include one or more products of collagen degradation, preferably an N-terminal telopeptide (NTX). In addition, methods for concurrently reducing the risk of both vertebral and non-vertebral bone fracture in a male human subject at risk of or having ossteoporosis are also disclosed, involving administration of human parathyroid hormone (amino acid sequence 1-34) without concurrent administration of an antiseropositive agent other than vitamin D or calcium.

### TECHNICAL FIELD

The present invention relates to a method for monitoring effects of administration of a parathyroid hormone by correlating such effects with levels of one or more markers of an activity of this hormone, and for using change in a biochemical marker of bone formation or turnover for predicting subsequent change in spine bone mineral density resulting from repetitive administration of a parathyroid hormone to a human subject. Specifically, the present method monitors the response of a serum or urine level of one or more markers of bone formation and resorption. In addition, the invention relates to methods for concurrently reducing the risk of both vertebral and non-vertebral bone fracture in a male human subject at risk of or having osteoporosis, by administering a parathyroid hormone parathyroid hormone without concurrent administration of an antiresorptive agent other than vitamin D or calcium.

### BACKGROUND OF THE INVENTION

Existing agents for treatment and prevention of bone trauma, diseases resulting in osteopenia and osteoporosis, such as estrogen, bisphosphonates, fluoride, or calcitonin can prevent bone loss and induce a 3-5% increase of bone mass by refilling the remodeling space, but net bone formation is net significantly stimulated. The retention of bone by inhibition of bone turnover may not be sufficient protection against fracture risk or other deleterious effects of conditions that increase risk of bone trauma. Anabolic agents that increase bone strength by stimulating bone formation preferentially may provide better protection against fracture in patients with established osteoporosis, but these agents do not treat or prevent several other indications that arise in osteoporosis.

Parathyroid hormone (PTH) is a secreted, 84 amino acid product of the mammalian parathyroid gland that controls serum calcium levels through its action on various tissues, including bone. The N-terminal 34 amino acids of bovine and human PTH (PTH(1-34)) is deemed biologically equivalent to the full length hormone. Other amino terminal fragments of PTH (including 1-31 and 1-38 for, example), or PTHrP (PTH-related peptide/protein) or analogues of either or both, that activate the PTH/PTHrP receptor (PTH1 receptor) have shown similar biologic effects on bone mass, although the magnitude of such effects may vary.

Studies in humans with various forms of PTH have demonstrated an anabolic effect on bone, and have prompted significant interest in its use for the treatment of osteoporosis and related bone disorders. The significant anabolic effects of PTH on bone, including stimulation of bone formation which results in a net gain in bone mass and/or strength, have been demonstrated in many animal models and in humans.

It is commonly believed that PTH administration in humans and in relevant animal models has a negative effect on cortical bone. In fact, naturally occurring increases in endogenous PTH which occur in the disorder hyperparathyroidism, result in thinning of cortical bone accompanied by an increase in connectivity and mass of trabecular bone. Past studies suggest that when Haversian cortical bone (found in humans and higher mammals) remodels under the influence of PTH, there will be a re-distribution of bone such that cortical bone mass and strength decrease, while trabecular bone increases in mass and strength. For example, in published clinical studies of administering PTH, cortical bone mass decreased after treatment with exogenous PTH and these findings have raised concern that tent with PTH will lead to reduced cortical bone mass and strength. One concern raised by such studies is that there would be a loss of total skeletal bone mass due to the loss of cortical bone. This is of high clinical relevance as, in osteoporosis, the greater loss of predominantly bone compared to loss of cortical bone, means that mechanical loading is predominantly borne by the remaining cortical bone. Continued loss of cortical bone would increase the fracture risk. Therefore, it is important that a therapeutic agent for osteoporosis maintain or increase a subject's residual cortical bone.

The effects of PTH on cortical bone have been investigated in nonhuman animals with Haversian remodeling, such as dogs, ferrets, sheep and monkeys, but sample sizes are typically too small for reliable statistical analysis. The impact of the changes induced by PTH treatment on mechanical properties of cortical bone in such animals remains unknown. Published studies of rodents have shown increased cortical bone mass during administration of PTH but a loss of this benefit after withdrawal of PTH. However, rodent cortical bone has a distinctly different structure from Haversian cortical bone, and remodels by surface appositional formation and resorption, rather than by intracortical remodeling of osteons. Furthermore, technological limitations in biomechanical testing on the relatively short bones of rodents give rise to artifacts of measurement when an agent, such as a PTH, alters bone geometry to thicken the bone. Such artifacts make extrapolation of rat cortical bone responses to those of humans or other animals with osteonal remodeling unreliable. Therefore, the existing data for animals, like humans, undergoing Haversian remodeling indicates that PTH may have an adverse impact on cortical bone, causing net loss of bone mass through depletion of cortical bone.

As a consequence, it has been a popular belief regarding the action of PTH that patients may not achieve sufficient benefit from admin ion of PTH to justify its use. In fact, it is commonly believed that patients require additional drug therapy to treat or prevent conditions or disorders that accompany osteoporosis or bone trauma. For example it is believed that osteoporosis patients require concurrent or subsequent treatment with an antiresorptive to minimize loss of bone induced by PTH. It was also believed that patients would require additional medications to reduce the incidence of or to treat disorders such as cancer, diabetes, a cerebrovascular disorder, and other disorders that affect subjects that might otherwise benefit from administration of PTH. In fact, this model requiring additional therapeutic agents for additional indications has been the basis for several clinical studies in women. For example, three clinical studies have used PTH in post-menopausal women undergoing concurrent therapy with calcitonin or estrogen, or in premenopausal women taking GnRH agonist, Synarel, for endometriosis. The opposing effects of estrogen and PTH on cortical bone turnover make it particularly difficult to observe effects of just PTH during combination therapy with these two agents.

Further, there are currently-no methods employing biological markers that are suitable for determining the course of therapy with parathyroid hormone. Although bone imaging or X-rays can be used to confirm treatment progress and outcomes, the use of markers provides an earlier and more accessible and economical alternative. Given the contradictory nature of beliefs regarding the various possible biological effects of therapy with parathyroid hormone, current knowledge could not provide a sensible prediction of the resulting levels of the numerous markers of these biological effects. For example, the rate of formation or degradation of the bone matrix can be assessed by measuring an enzymatic activity of bone-forming or -resorbing cells or by measuring bone matrix components released in to the circulation during bone formation or resorption. Bone formation can be assessed by measuring bone formation markers including serum osteocalcin, total and bone specific alkaline phosphatase, and procollagen I carboxyterminal extension peptide. Bone resorption can be assessed by measuring bone resorption markers including fasting urinary calcium, hydroxyproline, hydroxylysine glycosides, plasma tartrate-resistant acid phosphatase, and urinary excretion of the collagen pyridinium crosslinks and associated peptides such as N-telopeptide.

Although certain individual biological activities of a parathyroid hormone might be predicted to produce some effect on one of these markers in an in vitro system, there is a need for a method that correlates effective therapy using parathyroid hormone with levels of one or more biological markers.

### SUMMARY OF THE INVENTION

The present invention relates to a method for monitoring effects of administration of a parathyroid hormone by correlating such effects with levels of one or more markers of an activity of this hormone. Specifically, the present method monitors the response of a level of one or more markers of bone formation and resorption. Suitable markers of bone formation include one or more enzymes indicative of osteoblastic processes of bone formation, preferably bone specific alkaline phosphatase, and/or one or more products of collagen biosynthesis preferably a procollagen I C-terminal propeptide. Suitable markers of bone resorption include one or more products of collagen degradation, preferably an N-terminal telopeptide. In a preferred embodiment, the present method monitors the response of levels of one or more markers of bone formation and resorption including a bone specific alkaline phosphatase, a procollagen I C-terminal propeptide, N-telopeptide, free deoxypyridinoline or a combination thereof. The present method can distinguish administration of a parathyroid hormone from hormone replacement therapy or treatment with an antiresorptive agent.

In another aspect, the present invention provides a method for using change in a biochemical marker of bone formation for predicting subsequent change in spine bone mineral density resulting from repetitive administration of a parathyroid hormone to a human subject. In this method the biochemical marker of bone formation is an enzyme indicative of osteoblastic processes of bone formation or a product of collagen biosynthesis. This method comprises the steps of:
(a) determining the amount of difference for the subject between the level of the biochemical marker in a biological sample taken from the subject prior to administration of the hormone and the level in a sample taken after administration of hormone begins;
(b) comparing the amount of difference for the subject determined in step (a) with known amounts of difference for other human subjects determined as in step (a) to find a known amount of difference for other human subjects that is about the same as said that for the subject, wherein the parathyroid hormone has been administered to the other human subjects under the same conditions as for the subject of interest, and correlated amounts of subsequent change in spine bone mineral density resulting from administration of parathyroid hormone under these conditions are known for the known amounts of difference for other human subjects; and
(c) determining the known correlated amount of subsequent change in spine bone mineral density for the difference for the subject, thereby predicting that the subsequent change in spine bone mineral density due to administration of a parathyroid hormone to the subject will be that known correlated amount of subsequent change in spine bone mineral density.

In a preferred embodiment of this method, the repetitive administration is daily administration, the parathyroid hormone is hPTH(1-34), the biochemical marker of bone formation is the product of collagen biosynthesis in serum known as procollagen I C-terminal peptide (PICP) and the biological sample taken after administration of said hormone begins is taken about one month after administration of said hormone begins. This method may be used to predict change in spinal bone mineral density at a period of months or years, preferably about one year, after administration of the hormone begins.

According the invention, the method of predicting change in spine bone mineral density (dBMD) may further comprise a step in which the predicted dBMD determined in step (c) is adjusted for age and gender of the subjects, for base line PICP level of the subjects before administration of said hormone begs, and/or for the concentration of bone-specific alkaline phosphatase determined at about 3 moths after administration of hormone begins. Kits comprising reagents and instructions for using the above bone markers for prediction of spinal bone mineral density according to the methods of the invention also are provided by this invention.

The present invention also provides a method of treatment of osteoporosis or osteopenia, particularly in men, which is shown herein to substantially increase both vertebral and nonvertebral bone mineral density (BMD). Treatment of postmenopausal women with osteoporosis with parathyroid hormone (human PTH(1-34)) under the same conditions has been shown to concurrently reduce the risk of both vertebral and non-vertebral bone fracture. See PCT Patent Application No. PCT/US99/18961, published as WO 00/10596 on 2 march 20000. Given the similarities in responses to parathyroid hormone of men and women, in terms of both spinal and non-spinal BMD increases, as well as in bone marker responses described herein, concurrent reductions in the risk of both vertebral and non-vertebral bone fracture similar to those observed in women with osteoporosis are also expected in men with osteoporosis when the women and men are similarly treated with parathyroid hormone.

Accordingly, the present invention provides a method for concurrently reducing the risk of both vertebral and non-vertebral bone fracture in a male human subject at risk of or having osteoporosis, which may be either idiopathic or hypogonadal (age-related or other) in origin. This method comprises administering to the subject a parathyroid hormone, preferably the parathyroid hormone consisting of amino acid sequence 1-34 of human parathyroid hormone. This hormone is administered without concurrent administration of an antiresorptive agent other than vitamin D or calcium, in a daily dose in the range of at least about 15 μg to about 40 μg, for at least about 12 months up to about 3 years. In another embodiment, the invention provides an article of manufacture comprising packaging material and a pharmaceutical composition contained within that packaging material, where the composition comprises a parathyroid hormone consisting of amino acid sequence 1-34 of human parathyroid and the packaging material comprising printed matter which indicates that the composition is effective for concurrently reducing the risk of both vertebral and non-vertebral bone fracture in a male human subject at risk of or having osteoporosis when administered according to the present invention.

### BRIEF DESCRIPTION OF THE DRAWINGS

FIG. 1 illustrates the effect of administration of parathyroid hormone on levels of a bone specific alkaline phosphatase. Values for times greater than 12 months were determined from samples taken after discontinuation of PTH administration (median interval from discontinuation to sample was about 5-6 weeks).

FIG. 2 illustrates the effect of administration of parathyroid hormone on levels of a procollagen I C-terminal propeptide. Values for times greater than 12 months were after discontinuation of PTH (as in FIG. 1).

FIG. 3 illustrates the effect of administration of parathyroid hormone on levels of an N-telopeptide. Values for times greater than 12 months were after discontinuation of PTH (as in FIG. 1).

FIG. 4 illustrates the effects of administration of parathyroid hormone plus hormone replacement therapy or the administration of hormone replacement therapy on levels of a bone specific alkaline phosphatase. Values for times greater than 12 months were after discontinuation of PTH (as in FIG. 1).

FIG. 5 illustrates the effects of administration of parathyroid hormone plus hormone replacement therapy or the administration of hormone replacement therapy on levels of a procollagen I C-terminal propeptide. Values for times greater than 12 months were after discontinuation of PTH (as in FIG. 1).

FIG. 6 illustrates the effects of administration of parathyroid hormone plus hormone replacement therapy or the administration of hormone replacement therapy on levels of an N-telopeptide. Values for times greater than 12 months were after discontinuation of PTH (as in FIG. 1).

FIG. 7 illustrates the effects of administration of parathyroid hormone or administration of an antiresorptive agent on levels of a bone specific alkaline phosphatase. Values for times greater than 12 months were after continuation of PTH (as in FIG. 1).

FIG. 8 illustrates the effects of administration of parathyroid hormone or administration of an antiresorptive agent on levels of a procollagen IC-terminal propeptide. Values for times greater than 12 months were after discontinuation of PTH (as in FIG. 1).

FIG. 9 illustrates the effect of administration of parathyroid hormone or administration of an antiresorptive agent on levels of an N-telopeptide. Values for times greater than 12 months were after discontinuation of PTH (as in FIG. 1).

FIG. 10 illustrates the relationships between biochemical marker concentrations at 1 month and change in total lumbar spine BMD in females after 21 months of therapy. Individual predicted values from final treatment-response models are shown.

FIG. 11 illustrates the relationships between change from baseline for each biochemical marker at 1 month and change in total lumbar spine BMD in females after 21 months of therapy. Individual predicted values from final treatment-response models are shown.

FIG. 12 illustrates the final response-indicator model comparison of predicted total lumbar spine bone mineral density in females, showing that the goodness-of-fit of the model is represented by agreement between predicted BMD values, as well as by weighted residuals.

FIG. 13 illustrates the predicted effect of each covariate on the change in total lumbar spine BMD in females. Selected covariate values represent the mean, 5th, 25th, 75th and 95th percentile values from the patient population. Covariate of interest is varied while the remaining covariates are held constant at their mean.

FIG. 14 illustrates the range of predicted variability in total lumbar spine BMD response to hPTH(1-34) therapy for female patients in high and low responder categories. Shaded regions represent 25th and the 75th percentile BMD values calculated from 1000 simulation iterations for patients in the high and low responder categories. Covariate values are 5th and 95th percentile values from patient population.

FIG. 15 illustrates the relationships between biochemical marker concentrations at 1 month and change in femoral neck BMD in females after 21 months of hPTH(1-34) therapy. Individual predicted values from final treatment-response models are shown.

FIG. 16 illustrates the relationships between change from baseline for each biochemical marker at 1 month and change in femoral neck BMD in females after 21 months of hPTH(1-34) therapy. Individual predicted values from final treatment-response models are shown.

FIG. 17 illustrates the final hPTH(1-34) response-indicator model comparison of predicted femoral neck bone mineral density in females, showing that the goodness-of-fit of the model is represented by agreement between predicted BMD values, as well as by weighted residuals.

FIG. 18 illustrates the range of predicted variability in femoral neck BMD response to hPTH(1-34) therapy from the final response-indicator model in females. Shaded regions represent 25th and 75th percentile BMD values calculated from 1000 simulation iterations.

FIG. 19 illustrates effects of hPTH(1-34) therapy on lumbar spine BMD (mean percent change from baseline) by visit for an randomly assigned male patients.

FIG. 20 illustrates effects of hPTH(1-34) therapy on femoral BMD (mean percent change from baseline) by visit for all randomly assigned male patients.

FIG. 21 illustrates effects of hPTH(1-34) therapy on total hip BMD (mean percent change from baseline) by visit for all randomly assigned male patients.

FIG. 22 illustrates effects of hPTH(1-34) therapy on serum procollagen I carboxy-terminal propeptide (PICP) (mean percent change from baseline) by visit for all randomly assigned mare patients.

FIG. 23 illustrates effects of hPTH(1-34) therapy on serum bone-specific alkaline phosphatase (BSAP) (mean percent change from baseline) by visit for all randomly assigned male patients.

FIG. 24 illustrates effects of hPTH(1-34) therapy on urinary N-telopeptide/creatinine ratio (urinary NTX) (mean percent change from baseline) by visit for all randomly assigned male patients.

FIG. 25 illustrates an outline of the pharmacodynamic analyses performed in Example 6. Abbreviations: f( )=function of BMD=bone mineral density, BCM=biochemical marker, PICP=procollagen I carboxy-terminal propeptide, BSAP=bone-specific alkaline phosphatase, NTX=urinary N-telopeptide, DPD= urinary free deoxypyridinoline.

FIG. 26 illustrates the general process used for pharmacodynamic model development in each of the analyses of Example 6.

FIG. 27 illustrates the final neural network: comparison of observed and predicted change in total lumbar spine BMD for both females and males.

FIGS. 28-31 illustrate the final neural network predicted effect of covariates on change in total lumbar spine bone mineral density. Selected covariate values represent the mean, 5th, 25th, 75th, and 95th percentile values from the patient population. Covariate of interest is varied while the remaining covariates are held constant at their mean. Except where noted, patient is in 20-μg treatment group and has a baseline spine BMD of 0.85 g/cm². FIG. 28 illustrates the effect of treatment group (20 μg or 40 μg, left and right panels, lively) on the predicted change in spine BMD at 12 months based on change in PICP at 1 month. Separate curves for females and males are shown for each treatment group. FIG. 29 illustrates the effect of age at study entry (for females and males, left and right panels, respectively) on the predicted change in spine BMD at 12 months based an change in PICP at 1 month. FIG. 30 illustrates the effect of PICP at Baseline (pM) (for females and males, left and right panels, respectively) on the predicted change in spine BMD at 12 months based on change in PICP at 1 month. FIG. 31 illustrates the effect of BASP at 3 Months (pM) (for females and males, left and right panels, respectively) on the predicted change in spine BMD at 12 months based on change in PICP at 1 month.

FIGS. 32 and 33 illustrate change in PICP at 1 month versus individual predicted change in total lumbar spine bone mineral density at 12 months of treatment for female and male subjects (respectively) with Baseline PCIP less than 100 pM (left panels) or at least 100 pM (right panels). One data point not displayed on the plot for males with baseline PICP less than 100 pM: 498 pM vs. 193 g/cm². One data point not displayed on the plot for males with baseline PICP at least 100 pM: 533 pM vs. 0.175 g/cm².

FIG. 34 illustrates BSAP at 3 months versus individual predicted change in total lumbar spine bone mineral density at 12 months of treatment for both females (left panel) and males (right panel). Three data points not displayed on the plot for females: 52.3 pM vs. 0.098/cm², 65.2 pM vs. 0.055 g/cm², and 67.9 pM vs. 0.146 g/cm². One data point not displayed on the plot for males: 59.7 pM vs. 0.053 g/cm².

### DETAILED DESCRIPTION

Monitoring the Effects of Parathyroid Hormone

The present invention relates to a method for monitoring one or more effects of administration of a parathyroid hormone by correlating levels of one or more markers of an activity of this hormone. Specifically, the present method monitors the response of a level of one or more markers bone formation and resorption early in treatment as well as a profiles of change intermittently throughout treatment.

Suitable markers of bone formation include one or more enzymes indicative of osteoblastic processes of bone formation and/or one or more products of collagen biosynthesis and turnover. Enzymes indicative of osteoblastic processes include alkaline phosphatase, preferably bone specific alkaline phosphatase (BSAP), and the like. Products of collagen biosynthesis collagen, preferably type I collagen, an N-terminal propeptide from a collagen, a C-terminal propeptide from a collagen, and the like. A preferred product of collagen biosynthesis is a procollagen I C-terminal propeptide (PICP).

Suitable markers of bone resorption and turnover include one or more products of collagen degradation. Products of collagen degradation include product from a crosslinking domain of a collagen fibril (e.g. a hydroxyproline, a hydroxylysine, a pyridinoline, or a deoxypyridinoline), a collagen telopeptide, or the like. Collagen telopeptides include an N-terminal telopeptide and a C-terminal telopeptide. A preferred collagen telopeptide is an N-terminal telopeptide (NTX).

In a preferred embodiment, the present method monitors the response of levels of markers bone formation and resorption including BSAP, PICP, NTX, or a combination thereof, particularly early in treatment and then as needed over time.

The nature of this response after administration of the parathyroid hormone to a subject can correlate with the effect of the hormone on the subject. Steady or changing levels of these markers can indicate whether the parathyroid hormone is having a desired effect, no or a neutral effect, or an undesirable effect. Desirable effects of administering parathyroid hormone to a subject include increasing bone toughness and stiffness, decreasing incidence of fracture, decreasing incidence of diabetes and/or cerebrovascular disorder, decreasing incidence of cancer, increasing bone marrow quality, and the like.

Monitoring the effects of administering parathyroid hormone can occur throughout the period during which the parathyroid hormone is administered, and may start before administration of the parathyroid hormone. For example, a level of a marker can be determined concurrent with or before initiation of administration of a parathyroid hormone to establish a control level for the subject. The period of or during administration can be considered in three general phases, first, a period just after initiation of administration, second, a period subsequent to initiation of administration, and, third, a period of continuing administration. Although these periods can overlap, they are also sequential in the order listed.

The period just after initiation of administration typically starts at the time of initiation of administration and lasts for about 2 to about 15 weeks. The period subsequent to initiation of administration typically starts at the time of initiation of administration and lasts for about 6 to about 18 months, preferably about 12 to about 15 months. This period can also be considered to start at the end of the period just after initiation of administration. The period of continuing administration typically starts about 8 to about 12 months, preferably about 12 months, after initiation of administration and lasts until about 18 to about 36 months, preferably about 24 months, after initiation. The duration of these periods can also be envisioned as corresponding approximately to the duration of bone remodeling cycles. For example, the period just after initiation of administration can correspond to about the first remodeling cycle after initiation. The period subsequent to initiation of administration can generally correspond to the first and second remodeling cycles after initiation, or primarily to the second remodeling cycle. The period of continuing administration can generally correspond to the second and/or third remodeling cycles after initiation. Monitoring may also be continued after discontinuation of PTH treatment, to determine whether and when effects of the treatment on bone markers subside or disappear.

A desirable effect of administering parathyroid hormone can correlate with an increase in the level of a product of collagen biosynthesis, such as PICP, to an elevated level in the period just after initiation of administration. The level of a product of collagen biosynthesis, such as PICP, will typically pea during this period and decline until it approaches, comes near to, and perhaps returns to control or baseline levels during the period subsequent to initiation of administration. Typically during the period of continuing administration, the level of a product of collagen biosynthesis, such as PICP, reaches baseline or control level. An increase in level of a product of collagen biosynthesis, such as PICP, refers to an increase relative to a relevant control level, such as a pretreatment level in the subject, or relative to a level in a suitable, untreated control population.

A desirable effect of administering parathyroid hormone can correlate with an increase in the level of an enzyme indicative of osteoblastic processes of bone formation, such as BSAP, to an increasing or elevated level in the period just after initiation of administration. The level of an enzyme indicative of osteoblastic processes of bone formation, such as BSAP, can continue to increase and typically reaches and maintains an elevated level during the period subsequent to initiation of administration and during the period of continuing administration. After cessation of treatment, the level of an enzyme indicative of osteoblastic processes of bone formation, such as BSAP, decreases from its maintained, elevated level(s) and rapidly approaches or reaches baseline or control level. An increase in level of an enzyme indicative of osteoblastic processes of bone formation, such as BSAP, refers to an increase relative to a relevant control level, such as a pretreatment level in the subject, or relative to a level in a suitable, untreated control population.

A desirable effect of administering parathyroid hormone can correlate with a substantially constant or slightly increased level of a product of collagen degradation, such as NTX, during the period just after initiation of administration. The level of a product of collagen degradation, such as NTX, can continue to increase and typically reaches and maintains an elevated level during the period subsequent to initiation of administration. Typically during the period of continuing administration, the level of a product of collagen degradation, such as NTX, maintains this elevated level. An increase in level of a product of collagen degradation, such as NTX, refers to an increase relative to a relevant control level, such as a pretreatment level in the subject, or relative to a level in a suitable, untreated control population.

During the period just after initiation of administration a desirable effect of parathyroid hormone can result in an elevated level of a product of collagen biosynthesis, such as PICP; an increasing and possibly elevated level of an enzyme indicative of osteoblastic processes of bone formation, such as BSAP; a substantially constant or only slightly increased level of a product of collagen degradation, such as NIX; or a combination thereof.

During the period subsequent to initiation of administration a desirable effect of parathyroid hormone can result in a level of a product of collagen biosynthesis, such as PICP, below its peak or elevated level, preferably at or near a control level; an increasing or elevated level of an enzyme indicative of osteoblastic processes of bone formation, such as BSAP; a substantially constant, increasing, or elevated, preferably increasing or elevated, level of a product of collagen degradation, such as NIX; or a combination thereof.

During the period of continuing administration a desirable effect of parathyroid hormone can result in a level of a product of collagen biosynthesis, such as PICP, at or near a control or baseline level; an elevated level of an en enzyme indicative of osteoblastic processes of bone formation, such as BSAP; an elevated level of a product of collagen degradation, such as NTX; or a combination thereof. Observation of a desirable effect of parathyroid hormone administration during the period of continuing administration typically indicates that therapy has run its course, that the subject is likely not to benefit from additional administration of parathyroid hormone, that the subject is nearing completion of their desired response, and/or that discontinuation or at least temporary withdrawal of administration is desirable.

Observing a marker level indicating the desired response to administering parathyroid hormone typically leads to a decision to continue administration of the parathyroid hormone. Obtaining the desired response to administering parathyroid hormone can also lead to the decision to discontinue other possibly less effective therapies, such as hormone replacement therapy or antiresorptive therapy. For example, a subject may have been taking hormone replacement therapy or an antiresorptive agent before starting administration of parathyroid hormone. Due to some possible benefit of these previous therapies, the caregiver or subject may be reluctant to discontinue the previous therapies until they have evidence of a beneficial effect of administering parathyroid hormone. The present method can provide such evidence and support a decision to discontinue these previous therapies.

Failure to observe a marker level indicating the desired response to administering parathyroid hormone typically leads to a decision to alter administration of the hormone. Altering administration of the parathyroid hormone can include discontinuing administration or, alternatively increasing the dose of parathyroid hormone in an attempt to induce a desirable response. For example, failure to observe a marker level indicating the desired response to administering parathyroid hormone can indicate that the subject is not responding to or cannot respond to this therapy, and that administration can be discontinued. Alternatively, failure to observe a marker level indicating the desired response to administering parathyroid hormone can indicate increasing the dose of parathyroid hormone, which can then provide the desired response. Still another alternative is that failure to observe a marker level indicating the desired response to administering parathyroid hormone can indicate lack of compliance with the treatment regimen which therefore also should be considered and investigated prior to changing the treatment regimen.

The marker level is determined in a suitable biological sample from the subject and according to methods known to those of skill in the art. For example BSAP is typically determined from a serum sample. NTX is typically determined from a urine sample. The marker is typically determined employing a reagent such as an antibody, preferably a monoclonal antibody, recognizing and/or specific for the marker.

The present invention also encompasses a kit including reagents and other materials for practicing the method of the present invention. The kit can contain one or more containers, such as a vial, which contain, for example, one or more reagents for detecting a level of an enzyme indicative of an osteoblastic process of bone formation, such as BSAP, a product of collagen biosynthesis, such as PICP, and/or a product of collagen degradation, such as NTX. The container can also include, as required, a suitable carrier, either dried or in liquid form. The kit further includes instructions in the form of a label on the vial and/or in the form of an insert included in a box in which the vial is packaged, for carrying out the method of the invention. The instructions can also be printed on the box in which the vial is packaged. The instructions contain information such as amounts of reagents, order of mixing of reagents, steps for carrying out the method, incubation times and temperatures, or the like. It is anticipated that a worker in the field encompasses any doctor, nurse, or technician who might work in a medical facility or laboratory that would monitor administration of PTH.

Distinguishing Effects of Other Agents

The present method can also distinguish administration of a parathyroid hormone from administration of other agents employed against osteoporosis, such as hormone replacement therapy or treatment with an antiresorptive agent.

Hormone Replacement Therapy

Hormone replacement therapy (HRT) results in different changes in markers of bone formation and resorption than administration of a parathyroid hormone. Hormone replacement therapy includes any of the various regimens know to those of skill in the art. Hormone replacement therapy includes, for example, continuous and/or combined estrogen and progestin therapy for subjects having an intact uterus, or estrogen therapy for subjects without an intact uterus. Estrogen preparations include oral Premarin (e.g. 0.625 mg/day). Progestin preparations include oral Provera (e.g. 2.5 mg/day).

Suitable markers of bone formation for distinguishing administration of a parathyroid hormone from HRT include one or more enzymes indicative of osteoblastic processes of bone formation and/or one or more products of collagen biosynthesis. Enzymes indicative of osteoblastic processes include alkaline phosphatase, preferably bone specific alkaline phosphatase, and the like. Products of collagen biosynthesis include collagen, preferably type I collagen, an N-terminal propeptide from a collagen, a C-terminal propeptide from a collagen, and the like. A preferred product of collagen biosynthesis is a procollagen IC-terminal propeptide.

Suitable markers of bone resorption for distinguishing administration of a parathyroid hormone from HRT include one or more products of collagen degradation. Products of collagen degradation include a product from a crosslinking domain of a collagen fibril (e.g. a hydroxyproline, a hydroxylysine, a pyridinoline, or a deoxypyridinoline), a collagen telopeptide, or the like. Collagen telopeptides include an N-terminal telopeptide and a C-terminal telopeptide. A preferred collagen telopeptide is an N-terminal telopeptide.

In a preferred embodiment, the present method monitors the response of levels of markers bone formation and resorption including BSAP, PICP, NTX, or a combination thereof.

The patterns in makers of bone formation and resorption resulting from hormone replacement therapy are distinctly different from the patterns described above as resulting from administration of a parathyroid hormone. Through the course of up to about six months of hormone replacement therapy, levels of BSAP decrease. The BSAP level remains diminished for about the subsequent 12 months. Similarly, levels of PICP decrease during the first about 36 months of administration of hormone replacement therapy. The PICP level is then approximately constant but diminished for about the subsequent 12 months. Levels of NTX increase during the first about 3-6 months after initiation of hormone replacement therapy, followed by approximately steady but elevated levels over the subsequent about 12 months.

Antiresorptive Therapy

Antiresorptive therapy results in different changes in markers of bone formation and resorption than administration of a parathyroid hormone. Antiresorptive therapy includes any of the various regimens known to those of skill in the art, such as, for example, administration of alendronate (Fosamax®) (e.g. at 10 mg/day).

Suitable markers of bone formation for distinguishing administration of a parathyroid hormone from antiresorptive therapy include one or more enzymes indicative of osteoblastic processes of bone formation and/or one or more products of collagen biosynthesis. Enzymes indicative of osteoblastic processes include to alkaline phosphatase, preferably bone specific alkaline phosphatase, and the like. Products of collagen biosynthesis include collagen, preferably type I collagen, an N-terminal propeptide from a collagen, a C-terminal propeptide from a collagen, and the like. A preferred product of collagen biosynthesis is a procollagen IC-terminal propeptide.

Suitable markers of bone resorption for distinguishing administration of a parathyroid hormone from antiresorptive therapy include one or more products of collagen degradation. Products of collagen degradation include a product from a crosslinking domain of a collagen fibril (e.g. a hydroxyproline, a hydroxylysine, a pyridinoline, or a deoxypyridinoline), a collagen telopeptide, or the like. Collagen telopeptides include an N-terminal telopeptides and a C-terminal telopeptides. A preferred collagen telopeptide is an N-terminal telopeptide.

In a preferred embodiment, the present method monitors the response of levels of markers bone formation and resorption including BSAP, PICP, NTX, or a combination thereof.

The patterns in markers of bone formation and resorption resulting from antiresorptive therapy are distinctly different from the patterns described above as resulting from administration of a parathyroid hormone. Through the course of up to about six months of antiresorptive therapy, levels of BSAP decrease. The BSAP level is then approximately constant but diminished for about the subsequent 12 months. Similarly, levels of PICP decrease during the first about 3-6 months of administration of antiresorptive therapy. The PICP level is then approximately constant but diminished for about the subsequent 12 months. Levels of NTX decrease slightly during the first about 3-6 months after initiation of antiresorptive therapy, followed by approximately steady but decreased levels over the subsequent about 12 months.

Bone Trauma

The method of the invention is of benefit to a subject that may suffer or have suffered trauma to one or more bones. The method can benefit mammalian subjects, such as humans, horses, dogs, and cats, in particular, humans. Bone trauma can be a problem for racing horses and dogs, and also for household pets. A human can suffer any of a variety of bone traumas due, for example, to accident, medical intervention, disease, or disorder. Metastasis of cancer to the bone can result in a bone defect that puts the bone at risk of trauma. In the young, bone trauma is likely due to fracture, medical intervention to repair a fracture, or the repair of joints or connective tissue damaged, for example, through athletics. Other types of bone trauma, such as those from osteoporosis, degenerative bone disease (such as arthritis or osteoarthritis), hip replacement, or secondary conditions associated with therapy for other systemic conditions (e.g., glucocorticoid osteoporosis, buns or organ transplantation) are found most often in older people.

Bone trauma can be a problem for subjects at risk or having insufficient bone toughness and stiffness, bone fracture, diabetes and/or cerebrovascular disorder, cancer, insufficient bone marrow quality, and the like. For example, many subjects with the bone or metabolic disorders described above also are at risk of, have some risk factors for, or actually have insufficient bone toughness and stiffness, bone fracture, diabetes and/or cerebrovascular disorder, cancer, insufficient bone marrow quality, and the like. In particular, many women with or at risk of osteoporosis are also at risk of or have insufficient bone toughness and stiffness, bone fracture, diabetes and/or cerebrovascular disorder, cancer, insufficient bone marrow quality, and the like. The method of the invention can benefit these types of subjects.

Preferred subjects include a human, at risk for or suffering from osteoporosis or osteopenia. Risk factors for osteoporosis are known in the art and include hypogonadal conditions in men and women, irrespective of age, conditions, diseases or drugs that induce hypogonadism, nutritional factors associated with osteoporosis (low calcium or vitamin D being the most common), smoking, alcohol, drugs associated with bone loss (such as glucocorticoids, thyroxine, heparin, lithium, anticonvulsants etc.), loss of eyesight that predisposes to falls, space travel, immobilization, chronic hospitalization or bed rest, and other systemic diseases that have been linked to increased risk of osteoporosis. Indications of the presence of osteoporosis are known in the art and include radiological evidence of at least one vertebral compression fracture, low bone mass (typically at least 1 standard deviation below mean young normal values), and/or atraumatic fractures.

The method of the invention can benefit subjects suffering form, or at risk of, osteoporosis by, for example, increasing bone toughness and stiffness, decreasing incidence of fracture, decreasing incidence of diabetes and/or cerebrovascular disorder, decreasing incidence of cancer, increasing bone marrow quality, and the like. The present invention provides a method, in particular, effective to benefit a subject with or at risk of progressing to osteoporosis or patients in which spinal osteoporosis may be progressing rapidly. A typical woman at risk for osteoporosis is a postmenopausal woman or a premenopausal, hypogonadal woman. A preferred subject is a postmenopausal woman who is not concurrently taking hormone replacement therapy (HRT), estrogen or equivalent therapy, or antiresorptive therapy. The method of invention can benefit a subject at any stage of osteoporosis, but especially in the early and advanced stages.

Other subjects can also be at risk of or suffer bone trauma and can benefit from the method of the invention. For example, a wide variety of subjects at risk of one or more of the fractures identified above, can anticipate surgery resulting in bone trauma, or may undergo an orthopedic procedure that manipulates a bone at a skeletal site of abnormally low bone mass or poor bone structure, or deficient in mineral. For example, recovery of function after a surgery such as a joint replacement (e.g. knee or hip) or spine bracing, or other procedures that immobilize a bone or skeleton can improve due to the method of the invention. The method of the invention can also aid recovery from orthopedic procedures that manipulate a bone at a site of abnormally low bone mas or poor bone structure, which procedures include surgical division of bone, including osteotomies, joint replacement where loss of bone structure requires restructuring with acetabulum shelf creation and prevention of prosthesis drift, for example. Other suitable subjects for practice of the present invention include those suffering from hypoparathyroidism or kyphosis, who can undergo trauma related to, or caused by, hypoparathyroidism or progression of kyphosis.

Parathyroid Hormone

As active ingredient, the composition or solution may incorporate the full length, 84 amino acid form of parathyroid hormone, particularly the human form, hPTH (1-84), obtained either recombinantly, by peptide synthesis or by extraction from human fluid. See, for example, U.S. Pat. No. 5,208,041, incorporated herein by reference. The amino acid sequence for hPTH (1-84) is reported by Kimura et al. in Biochem. Biophys. Res. Comm., 114(2):493.

The composition or solution may also incorporate as active ingredient fragments or variants of fragments of human PTH or of rat, porcine or bovine PTH is that have human PTH activity as determined in the ovariectomized rat model of osteoporosis reported by Kimmel et al., Endocrinology, 1993, 32(4):1577.

The parathyroid hormone fragments desirably incorporate at least the first 28 N-terminal residues, such as PTH(1-28), PTH(1-31), PTH(1-34), PTH(1-37), PTH(1-38) and PTH(1-41). Alternatives in the form of PTH variants incorporate from 1 to 5 amino acid substitutions that improve PTH stability and half-life, such as the replacement of methionine residues at positions 8 and/or 18 with leucine or other hydrophobic amino acid that improves PTH stability against oxidation and the replacement of amino acids in the 25-27 region with trypsin-insensitive amino acids such as histidine or other amino acid that improves PTH stability against protease. Other suitable forms of PTH include PTHrP, PTHrP(1-34), PTHrP(1-36) and analogs of PTH or PTHrP that activate the PTH1 receptor. These forms of PTH are embraced by the term “parathyroid hormone” as used generically herein. The hormones may be obtained by known recombinant or synthetic methods, such as described in U.S. Pat. Nos. 4,086,196 and 5,556,940, incorporated herein by reference.

The preferred hormone is human PTH(1-34). Stabilized solutions of human PTH(1-34), such as recombinant human PTH(1-34) (rhPTH(1-34), that can be employed in the present method are described in U.S. patent application Ser. No. 60/069,075, incorporated herein by reference. Crystalline forms of human PTH(1-34) that can be employed in the present method are described in U.S. patent application Ser. No. 60/069,875, incorporated herein by reference.

Administering Parathyroid Hormone

A parathyroid hormone can typically be administered parenterally, preferably by subcutaneous injection, by methods and in formulations well known in the art. Stabilized formulations of human PTH(1-34) that can advantageously be employed in the present method are described in U.S. patent Application Ser. No. 60/069,075, incorporated herein by reference. This patent application also describes numerous other formulations for storage and administration of parathyroid hormone. A stabilized solution of a parathyroid hormone can include a stabilizing agent, a buffering agent, a preservative, and the like.

The stabilizing agent incorporated into the solution or composition includes a polyol which includes a saccharide, preferably a monosaccharide or disaccharide, e.g., glucose, trehalose, raffinose, or sucrose; a sugar alcohol such as, for example, mannitol, sorbitol or inositol, and a polyhydric alcohol such as glycerine or propylene glycol or mixtures thereof. A preferred polyol is mannitol or propylene glycol. The concentration of polyol may range from about 1 to about 20 wt-%, preferably about 3 to 10 wt-% of the total solution.

The buffering agent employed in the solution or composition of the present invention may be any acid or salt combination which is pharmaceutically acceptable and capable of maintaining the aqueous solution at a pH range of 3 to 7, preferably 3-6. Useful buffering systems are, for example, acetate, tartrate or citrate sources. Preferred buffer systems are acetate or tartrate sources, most preferred is an acetate source. The concentration of buffer may be in the range of about 2 mM to about 500 mM, preferably about 2 mM to 100 mM.

The stabilized solution or composition of the present invention may also include a parenterally acceptable preservative. Such preservations include, for example, cresols, benzyl alcohol, phenol, benzalkonium chloride, benzethonium chloride, chlorobutanol, phenylethyl alcohol, methyl paraben, propyl paraben, thimerosal and phenylmercuric nitrate and acetate. A preferred preservative is m-cresol or benzyl alcohol; most preferred is m-cresol. The amount of preservative employed may range from about 0.1 to about 2 wt-%, preferably about 0.3 to about 1.0 wt-% of the total solution.

Thus, the stabilized PTH solution can contain mannitol, acetate and m-cresol with a predicted shelf-life of over 15 months at 5° C.

The parathyroid hormone compositions can, if desired, be provided in a powder form containing not more than 2% water by weight, that results from the freeze-drying of a sterile, aqueous hormone solution prepared by mixing the selected parathyroid hormone, a buffering agent and a stabilizing agent as above described. Especially useful as a buffering agent when preparing lyophilized powders is a tartrate source. Particularly useful stabilizing agents include glycine, sucrose, trehalose and raffinose.

In addition, parathyroid hormone can be formulated with typical buffers and excipients employed in the art to stabilize and solubilize proteins for parenteral administration. Art recognized pharmaceutical carriers and their formulations are described in Martin, “Remington's Pharmaceutical Sciences,” 15th Ed.; Mack Publishing Co., Easton (1975). A parathyroid hormone can also be delivered via the lungs, mouth, nose, by suppository, or by oral formulations.

The parathyroid hormone is formulated for administering a dose effective for increasing bone toughness and stiffness, decreasing incidence of fracture, decreasing incidence of diabetics and/or cerebrovascular disorder, decreasing incidence of cancer, increasing bone marrow quality, and the like. Preferably, a subject receiving parathyroid hormone also receives effective doses of calcium and vitamin D, which can enhance the effects of the hormone. An effective dose of parathyroid hormone is typically greater than about 5 μg/day although, particularly in humans, it can be as large at about 10 to about 40 μg/day, or larger as is effective for increasing bone toughness and stiffness, decreasing incidence of fracture, decreasing incidence of diabetes and/or cerebrovascular disorder, decreasing incidence of cancer, increasing bone marrow quality, and the like. A subject suffering from hypoparathyroidism can require additional or higher doses of a parathyroid hormone; such a subject also requires replacement therapy with the hormone. Doses required for replacement therapy in hypoparathyroidism are known in the art. In certain it relevant effects of PTH can be observed at doses less than about 5 μg/day, or even less than about 1 μg/day.

The hormone can be administered regularly (e.g., once or more each day or week), intermittently (e.g. irregularly during a day or week), or cyclically (e.g., regularly for a period of days or weeks followed by a period without administration). Preferably PTH is administered once daily for 1-7 days per week over a period ranging from 3 months for up to 3 years in osteoporotic patients. Preferably, cyclic administration includes administering a parathyroid hormone for at least 2 remodeling cycles and withdrawing parathyroid hormone for at least 1 remodeling cycle. Another preferred regime of cyclic administration includes administering the parathyroid hormone for at least about 12 to about 24 months and withdrawing parathyroid hormone for at least 6 months. Typically, the benefits of administration of a parathyroid hormone persist after a period of administration. The benefits of several months of administration can persist for as much as a year or two, or more, without additional administration.

Additional aspects of administration of a parathyroid hormone are described in U.S. patent application No. 60/099,746 and PCT Patent Application No. PCT/US99/18961, which claim priority to the U.S. application, the disclosure of which are incorporated herein by reference.

Uses of Formulations of a Parathyroid Hormone

A kit including the present pharmaceutical compositions can be used with the methods of the present invention. The kit can contain a vial which contains a formulation of the present invention and suitable carriers, either dried or in liquid form. The fit further includes ins ons in the form of a label on the vial and/or in the form of an inset included in a box in which the vial is packaged, for the use and administration of the compounds. The instructions can also be pled on the box in which the vial is packaged. The instructions contain information such as sufficient dosage and administration information so as to allow a worker in the field to administer the drug. It is anticipated that a worker in the field encompasses any doctor, nurse, or technician who might administer the drug.

A PTH pharmaceutical composition for administering in the present invention can include a formulation of one or more parathyroid hormones, such as human PTH(1-84) or human PTH(1-34), and that is suitable for parenteral administration. A formulation of one or more parathyroid hormones, such as human PTH(1-84) or human PTH(1-34), can be used for manufacturing a composition or medicament suitable for administration by parenteral administration. The PTH composition can be produced by any of a variety of methods for manufacturing compositions including a formulation of one or more parathyroid hormones, such as human PTH(1-84) or human PTH(1-34), in a form that is suitable for parenteral administration. For example, a liquid or solid formulation can be manufactured in several ways, using conventional techniques. A liquid formulation can be manufactured by dissolving the one or parathyroid hormones, such as human PTH(1-84) or human PTH(1-34), in a suitable solvent, such as water, at an appropriate pH, including buffers or other excipients, for example to form one of the stabilized solutions described hereinabove.

The examples which follow are illustrative of the invention and are not intended to be limiting.

### EXAMPLE 1

### Monitoring Administration of rhPTH(1-34) to Humans by Monitoring Markers of Bone Formation and/or Resorption

Number of Subjects:
   rhPTH(1-34): 1093 enrolled, 848 finished.
   Placebo: 544 enrolled, 447 finished.
Diagnosis and Inclusion Criteria: Women ages 30 to 85 years, postmenopausal for a minimum of 5 years, with a minimum of one moderate or two mild atraumatic vertebral fractures.
Dosage and Administration:
   Test Product (blinded)
   rhPTH(1-34): 20 μg/day, given subcutaneously
   rhPTH(1-34): 40 μg/day, given subcutaneously
   Reference Therapy (blinded)
   Placebo study material for injection
Duration of Treatment:
   rhPTH(1-34): 17-23 months (excluding 6-month run-in phase)
   Placebo: 17-23 months (excluding 6-month run-in phase)
Criteria for Evaluation: Spine x-ray; serum biological markers (calcium, bone-specific alkaline phosphatase, procollagen I carboxy-terminal propeptide); urine markers (calcium, N-telopeptide, free deoxypyridinoline); 1,25-dihydroxyvitamin D; bone mineral density: spine, hip, wrist, and total body: height; population pharmacokinetics; bone biopsy (selected study sites).

Patient Characteristics

| | Placebo | | PTH-20 | | PTH-40 | | |
|---|---|---|---|---|---|---|---|
| | (N = 544) | | (N = 541) | | (N = 552) | | p-value |
| Caucasian | 98.9% | | 98.9% | | 98.4% | | 0.672 |
| Age | 69.0 ± 7.0 | | 69.5 ± 7.1 | | 69.9 ± 6.8 | | 0.099 |
| Years post menopausal | 20.9 ± 8.5 | | 21.5 ± 8.7 | | 21.8 ± 8.2 | | 0.273 |
| Hysterectomized | 23.8% | | 23.1% | | 21.6% | | 0.682 |
| Uterus + 0 or 1 ovary | 57 | | 51 | | 58 | | |
| Uterus + 2 ovaries | 61 | | 57 | | 51 | | |
| Unknown | 11 | | 17 | | 10 | | |
| Previous osteoporosis | 14.9% | | 15.5% | | 13.0% | | 0.479 |
| drug use | | | | | | | |
| Baseline spine BMD | 0.82 ± 0.17 | | 0.82 ± 0.17 | | 0.82 ± 0.17 | | >0.990 |
| Baseline # of vert. fx | | | | | | | |
| 0 | 54 | (10.4%) | 45 | (8.8%) | 54 | (10.1%) | |
| 1 | 144 | (27.8%) | 159 | (31.1%) | 169 | (31.6%) | |
| 2 | 128 | (24.7%) | 128 | (25.0%) | 125 | (23.4%) | |
| 3 | 75 | (14.5%) | 67 | (13.1%) | 81 | (15.1%) | |
| 4 | 59 | (11.4%) | 49 | (9.6%) | 45 | (8.4%) | |
| 5 | 28 | (5.4%) | 31 | (6.1%) | 21 | (3.9%) | |
| 6 | 13 | (2.5%) | 20 | (3.9%) | 25 | (4.7%) | |
| 7 | 6 | (1.2%) | 7 | (1.4%) | 10 | (1.9%) | |
| 8 | 9 | (1.7%) | 5 | (1.0%) | 3 | (0.6%) | |
| 9 | 1 | (0.2%) | 0 | | 2 | (0.4%) | |
| 10 | 1 | (0.2%) | 1 | (0.2%) | 0 | | |
| Unspecified | 26 | | 29 | | 17 | | >0.990 |

Methods

Measures of BSAP, PICP, and NTX levels were determined for each patient through the course of therapy, for example, at 0, 1, 3, 6, 12, 21 and 24 months after the initiation of administration of parathyroid hormone. Parathyroid hormone treatment was discontinued after 17-23 months. The percent change (relative to the initial “0” month levels) for each marker was determined for each patient and is reported in the Figures. The overall changes observed in the 20 μg/day PTH-treated patient population, the 40 μg/day PTH-treated treated patient population, and the placebo patient population were determined by methods known to those of skill in the arts.

Results

Data from this clinical trial including a total of 1637 women treated with recombinant human parathyroid hormone (1-34), rhPTH(1-34) 0, 20, or 40 μg/day, and supplemented with vitamin D and calcium, for 17-23 months, showed results reported below.

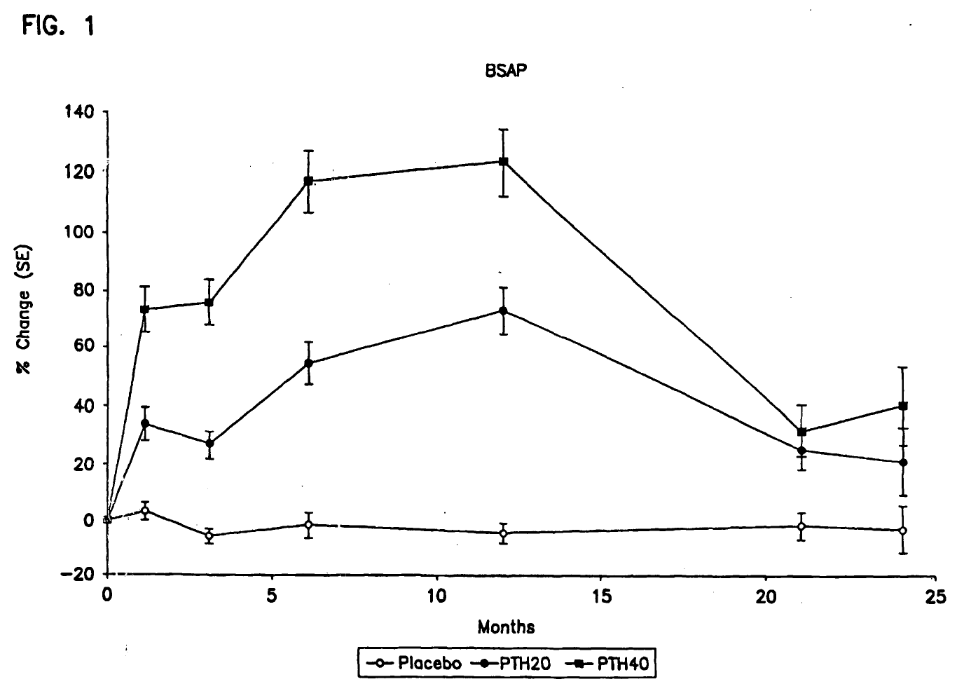

FIG. 1 illustrates data showing the percent change (and standard error, SE) over time of BSAP serum levels in patients administered 20 μg/day PTH, 40 μg/day PTH, and to placebo. BSAP is a marker for bone formation, and thus increases in BSAP levels correlate with increases in bone formation. As shown in FIG. 1, the percent change in BSAP levels began to increase as early as one month and continued to increase reaching a peak at about 6 to about 12 months after initiation of PTH treatment in both the 20 μg/day PTH and the 40 μg/day PTH populations, and then maintaining an elevated level. No such increase in BSAP level was observed in patients receiving placebo. At about 5-6 weeks following termination of PTH treatment (at 17-23 after initiation of treatment), which was about 21-24 months after initiation of PTH therapy, the level of BSAP in patients receiving PTH returned to a level at or slightly higher than placebo control levels (FIG. 1).

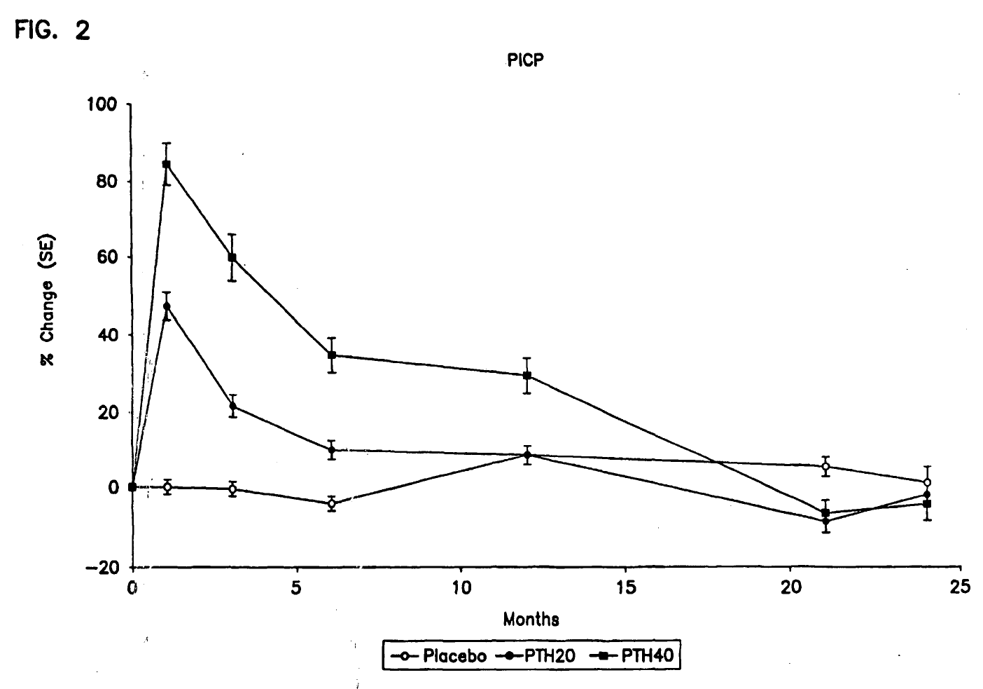

FIG. 2 illustrates data showing the percent change (and standard error, SE) over time of PICP serum levels in patients administered 20 μg/day PTH, 40 μg/day PTH, and to placebo. PICP is a marker for bone formation, and thus increases in PICP levels correlate with increases in bone formation. As shown in FIG. 2, the percent change in PICP levels increased rapidly and reached a peak within about one or two months after initiation of PTH treatment in both the 20 μg/day PTH and the 40 μg/day PTH populations. However, no such increase was observed in patients receiving placebo. After the PICP levels peaked, they slowly returned to levels at or near control levels, while maintaining elevated levels for some time. At about 12 months of treatment, the PICP levels in patients administered 20 μg/day PTH were at or near control levels. At about 5-6 weeks following termination of PTH treatment (at 17-23 after initiation of treatment), which was about 21-24 months after initiation of PTH therapy, the level of PICP in all PTH-treated patients returned to a level about the same as placebo controls.

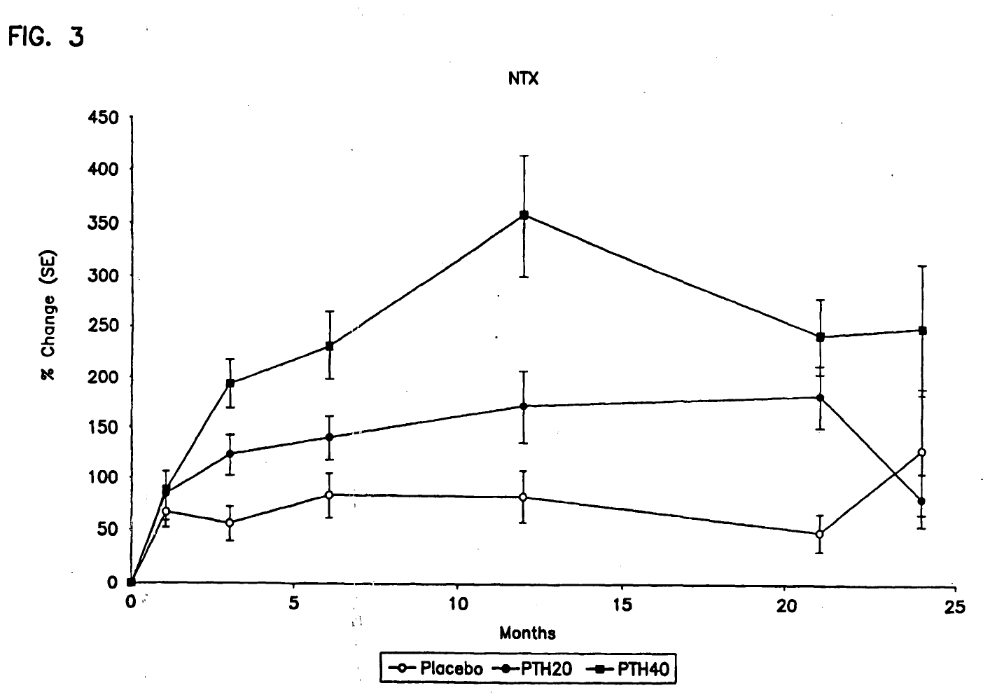

FIG. 3 illustrates data showing the percent change (and standard error, SE) over time of NTX urine levels in patients administered 20 μg/day PTH, 40 μg/day PTH, and placebo. NTX is a marker for bone resorption, and thus increases in NTX levels correlate with increases in bone resorption. As shown in FIG. 3, the percent change in NTX levels began to increase in both PTH treated and control-subjects as early as one month into the study. That is, all patients remained at control levels for at least about 1 month after treatment began. After one month the percent change in NTX in placebo patients did not further increase. However, in the 20 μg/day PTH and the 40 μg/day PTH populations, the percent change in NTX levels increased steadily until about 12 months after treatment initiation. At about 56 weeks following termination of PTH treatment (at 17-23 after initiation of treatment), which was about 21-24 months after initiation of PTH therapy, the percent change in NTX levels declined and returned to levels similar to those observed in the placebo treated group.

In summary, these data show that monitoring the selective regulation of one or more of 3 markers, an enzyme indicative of osteoblastic processes of bone formation, BSAP, a product of collagen biosynthesis, PICP, and a product of collagen degradation, NTX, can be used to determine responders and duration of treatment with parathyroid hormone.

Discussion

Based on the data presented above, monitoring markers of bone turnover and resorption including an enzyme indicative of osteoblastic processes of bone formation, BSAP, a product of collagen biosynthesis, PICP, and/or a product of collagen degradation, NTX, can be used to establish efficacy of treatment, identify responders, and determine duration of treatment. Changing profiles of bone markers can be used to establish efficacy of treatment or to monitor actions of PTH and to determine duration of therapy in patients whose skeletons are at risk of fracture. For example, early in treatment a rise in a product of collagen biosynthesis, PICP, no change in a product of collagen degradation, NTX, and/or some increase in an enzyme indicative of osteoblastic processes of bone formation, BSAP, can identify those patients that respond to treatment. By way of further example, a rise and maintained increase in an enzyme indicative of osteoblastic processes of bone formation, BSAP, normal level of a product of collagen biosynthesis, PICP, and/or normal or progressively increasing level of a product of collagen degradation, NTX over a period of months, can be used to confirm that patients continue to respond to PTH and that bone formation is active. Additionally, maintenance of elevated product of collagen degradation, NTX, after about 12-18 months; normal level of a product of collagen biosynthesis, PICP, and/or elevated enzyme indicative of osteoblastic processes of bone formation, BSAP can be used to signal that therapy has run its course.

### EXAMPLE 2

### Monitoring Administration of rhPTH(1-34) to Humans also Receiving Hormone Replacement Therapy by Monitoring Markers of Bone Formation and/or Resorption

Number of Subjects:
   rhPTH(1-34) plus hormone replacement therapy (HRT) (estrogen±progesterone): 122 enrolled, 91 finished.
   Control, hormone replacement therapy (estrogen±progesterone) without PTH: 125 enrolled, 105 finished.
Diagnosis and Inclusion Criteria: Women aged 62±8 years, postmenopausal for 15±8 years, selected for a baseline spine bone mineral density of 0.9±0.15 and a T value of −1.8.
Dosage and Administration:
   Test Product (blinded)
   rhPTH(1-34): 40 μg/day, given subcutaneously plus hormone replacement therapy (estrogen±progesterone). Subjects continued their prestudy hormone replacement therapy, maintained an HRT regimen consistent with local medical practices, took continuous/combined estrogen and progestin therapy using oral Premarin (0.625 mg/day) and oral Provera (2.5 mg/day) (intact uterus), or took estrogen therapy using oral Premarin (0.625 mg/day) (without intact uterus).
   Reference (Control) Therapy (Blinded)
   Hormone replacement therapy (estrogen±progesterone). Subjects continued their prestudy hormone replacement therapy, maintained an HRT regimen consistent with local medical practices, took continuous/combined estrogen and progestin therapy using oral Premarin (0.625 mg/day) and oral Provera (2.5 mg/day) (intact uterus), or took estrogen therapy using oral Premarin (0.625 mg/day) (without intact uterus).
Duration of Treatment:
   rhPTH(1-34): up to 18 months
   Control: up to 18 months
Criteria for Evaluation: Spine x-ray, serum biological makers (calcium, bone-specific alkaline phosphatase procollagen I carboxy-terminal propeptide); urine markers (calcium, N-telopeptide, fee deoxypyridinoline); 1,25-dihydroxyvitamin D; bone mineral density: spine, hip, wrist, and total body.

Patient Characteristics

| | Control | PTH-40 plus |
|---|---|---|
| | (HRT) | HRT |
| | (N = 125) | (N = 122) |
| Caucasian | 66.4% | 67.2% |
| Hispanic | 31.2% | 32.0% |
| Age | 61.1 ± 7.4 | 61.9 ± 7.6 |
| Years post menopausal | 14.5 ± 7.9 | 15.0 ± 8.1 |
| Hysterectomized | 40.0% | 48.4% |
| Uterus + 0 or 1 ovary | 20 | 34 |
| Uterus + 2 ovaries | 27 | 31 |
| Unknown | 3 | 4 |
| Previous osteoporosis drug | 48.8% | 50.0% |
| use | | |
| Baseline spine BMD | 0.91 ± 0.15 | 0.90 ± 0.15 |

Methods

Measures of BSAP, PICP, and NTX levels were determined for each patient through the course of therapy generally according to methods described above in Example 1.

Results

Data from this clinical trial including a total of 247 women treated with recombinant human parathyroid hormone (1-34), rhPTH(1-34) 0 or 40 μg/day plus hormone replacement therapy, and supplemented with vitamin D and calcium, for up to 18 months, showed results reported below.

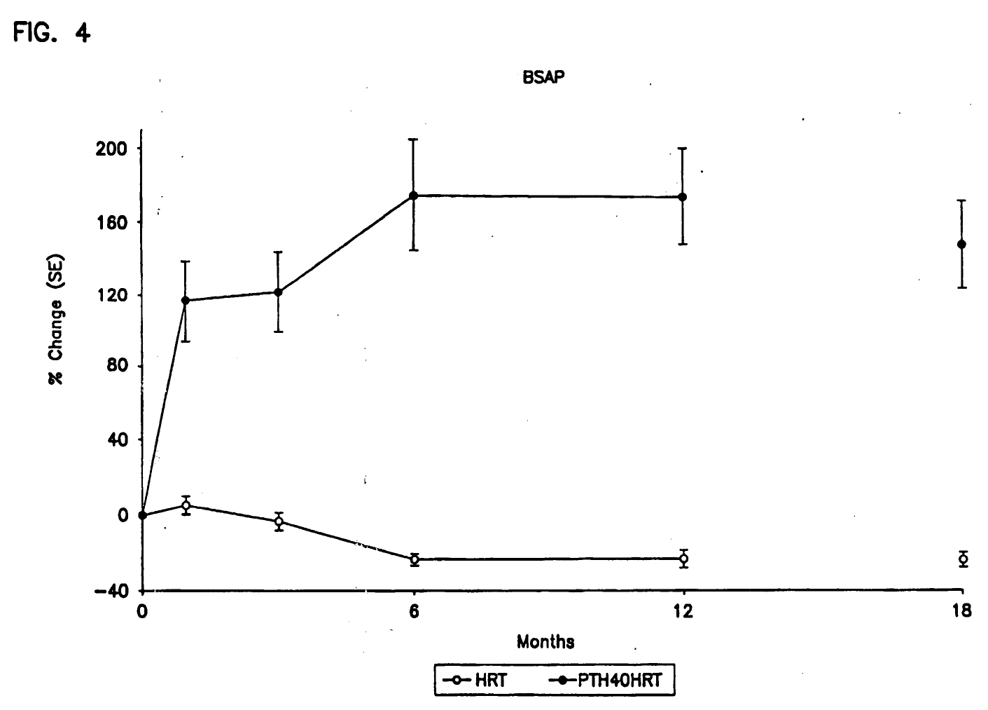

FIG. 4 illustrates data showing the percent change (and standard error, SE) over time of BSAP serum levels in patients administered 40 μg/day PTH plus HRT or just HRT. BSAP is a marker for bone formation, and thus increases in BSAP levels correlate with increases in bone formation. As shown in FIG. 4, the percent change in BSAP levels began to increase as early as one month and continued to increase reaching a peak at about 6 to about 12 months after initiation of PTH treatment in the 40 μg/day PTH population. At about 5-6 weeks following termination of PTH treatment (at 18 months from treatment initiation), BSAP in PTH-treated patients maintained an elevated level. No such increase in BSAP level was observed in patients receiving only HRT (FIG. 4).

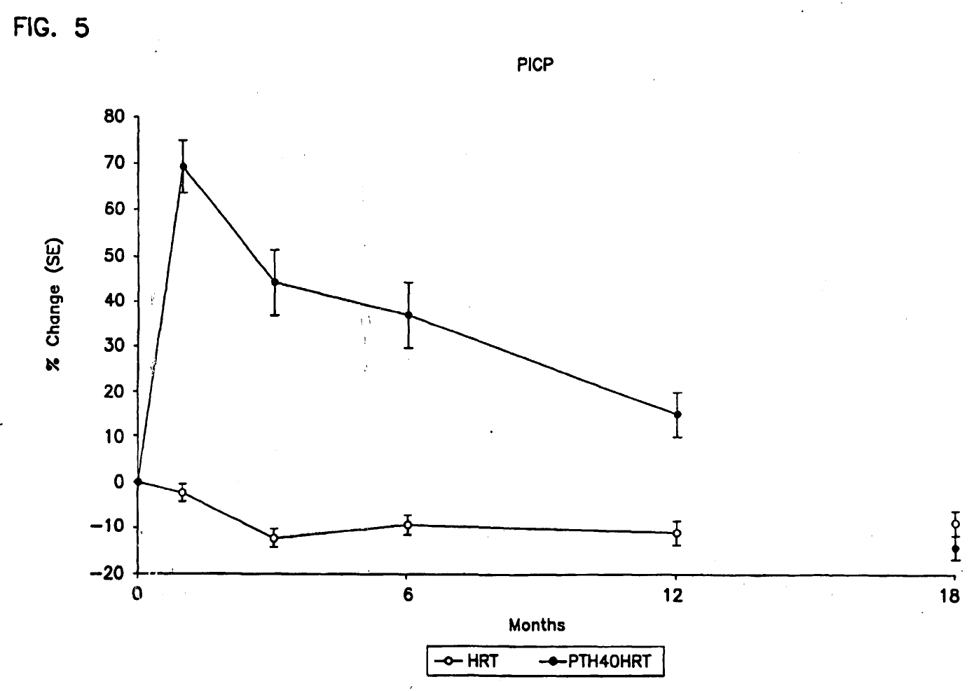

FIG. 5 illustrates data showing the percent change (and standard error, SE) over time of PICP serum levels in patients administered 40 μg/day PTH plus HRT, or just HRT. PICP is a marker for bone formation, and thus increases in PICP levels correlate with increases in bone formation. As shown in FIG. 5, the percent change in PICP levels increased rapidly and reached a peak within about one or two months after initiation of PTH treatment in the 40 μg/day PTH population. However, no such increase was observed in patients receiving only HRT. After, the PICP levels peaked, they slowly returned to levels at or near control levels, while maintaining elevated levels for some time. After about 12 months of tent, the PICP levels of PTH-treated patients approached control levels. At about 5-6 weeks following termination of PTH treatment (at 18 months from treatment initiation), PICP levels were the same as HRT controls (FIG. 5).

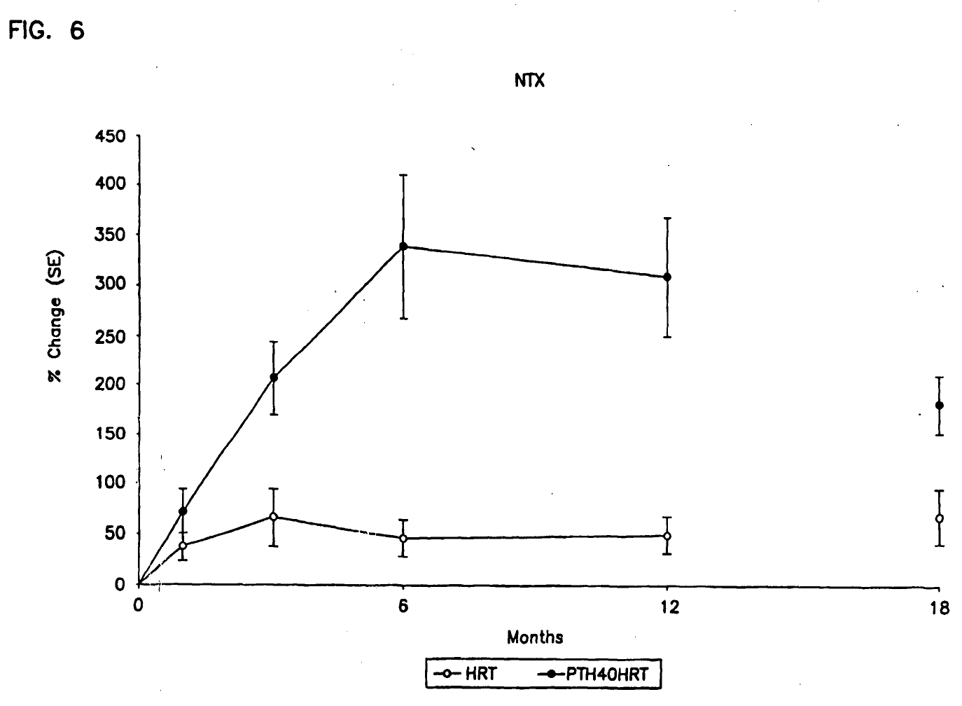

FIG. 6 illustrates data showing the percent change (and standard error, SE) over time of NTX urine levels in patients administered 40 μg/day PTH plus HRT, or is just HRT. NTX is a marker for bone resorption, and thus increases in NTX levels correlate with increases in bone resorption. As shown in FIG. 6, the percent change in NTX levels began to increase in both PTH treated and control subjects as early as one month into the study. That is, all patients remained at or near control levels for at least about 1 month after treatment began. After one month the percent change in NTX in control patients did not undergo significant further increase. However, in the 40 μg/day PTH population, the percent change in NTX levels increased steadily until about 6 months after treatment initiation and remained at about the same high level at 12 months. At about 5-6 weeks following termination of PTH treatment (at 18 months from treatment initiation), the percent change in NTX levels had declined and approached levels similar to those observed in the control group.

In summary, these data show that monitoring the selective regulation of one or more of 3 markers, an enzyme indicative of osteoblastic processes of bone formation, BSAP, a product of collagen biosynthesis, PICP, and/or a product of collagen degradation, NTX, can be used to determine responders and duration of treatment with parathyroid hormone. Further, these data show that monitoring the selective regulation one or more of 3 markers, an enzyme indicative of osteoblastic processes of bone formation, BSAP, a product of collagen biosynthesis, PICP, and/or a product of collagen degradation, NTX, can be used to distinguish administration of parathyroid hormone from administration of HRT.

Discussion

Based on the data presented above, monitoring of one or more markers of bone turnover including an enzyme indicative of osteoblastic processes of bone formation, BSAP, a product of collagen biosynthesis, PICP, and/or a product of collagen degradation, NTX, can be used to establish efficacy of treatment, identify responders, and determine duration of treatment for a regimen including administration of both PTH and hormone replacement therapy. This is in contrast to hormone replacement therapy, which resulted in significantly different patterns in these markers. Thus, the method distinguished between therapy with HRT and with parathyroid hormone. The method also effectively monitored administration of parathyroid hormone in patients also taking HRT.

Changing profiles of bone markers can be used during concurrent HRT to establish efficacy of treatment or to monitor actions of PTH and to determine duration of PTH therapy in patients whose skeletons are at risk of fracture. For example, early in treatment a rise in a product of collagen biosynthesis, PICP, no change in a product of collagen degradation, NTX, and/or some increase in an enzyme indicative of osteoblastic processes of bone formation, BSAP, can identify those patients that respond to PTH treatment. By way of further example, a rise and maintained increase in an enzyme indicative of osteoblastic processes of bone formation, BSAP, normal level of a product of collagen biosynthesis, PICP, and/or normal or progressively increasing product of collagen degradation, NTX, over a period of months, can be used to confirm that patients continue to respond to PTH and that bone formation is active. Additionally, maintenance of elevated product of collagen degradation, NTX, after about 12-18 months, normal level of a product of collagen biosynthesis, PICP, and/or elevated enzyme indicative of osteoblastic processes of bone formation, BSAP, can be used to signal that PTH therapy has run its course.

### EXAMPLE 3

### Monitoring Administration of rhPTH(1-34) to Humans by Monitoring Markers of Bone Formation and/or Resorption and Comparison to Treatment with an Antiresorptive

Number of Subjects:
   rhPTH(1-34): 73 enrolled, 51 finished.
   Alendronate (Fosamax®): 73 enrolled, 57 finished.
Diagnosis and Inclusion Criteria: Women aged 65±8 years, postmenopausal for 19±19 years, selected for a baseline spine bone mineral density of 0.8±0.1 and a T value of −2.2.
   Dosage and Administration:
   Test Product (blinded)
   rhPTH(1-34): 40 μg/day, given subcutaneously
   Reference (Control) Therapy (blinded)
   Alendronate (Fosamax®): 10 mg per patient per day
Duration of Treatment:
   rhPTH(1-34): 12-18 months, with follow up from time of withdrawal of drug to 18 months of study.
   Alendronate: 12-18 months, with follow up from time of withdrawal of drug to 18 months of study.
Criteria for Evaluation: Spine x-ray, serum biological markers (calcium, bone-specific alkaline phosphatase, procollagen I carboxy-terminal propeptide); urine makers (calcium, N-telopeptide, fir deoxypyridinoline); 1,25-dihydroxyvitamin D; bone mineral density spine, hip, wrist, and total body.

Patient Characteristics

| | Alendronate | PTH-40 |
|---|---|---|
| | (N = 125) | (N = 122) |
| Caucasian | 82.2% | 82.2% |
| Hispanic | 16.4% | 16.4% |
| Age | 64.9 ± 8.6 | 65.9 ± 7.8 |
| Years post menopausal | 19.2 ± 9.7 | 18.4 ± 9.1 |
| Hysterectomized | 34.2% | 19.2% |
| Uterus + 0 or 1 ovary | 13 | 7 |
| Uterus + 2 ovaries | 12 | 5 |
| Unknown | 0 | 2 |
| Previous osteoporosis drug | 5.5% | 11.0% |
| use | | |
| Baseline spine BMD | 0.79 ± 0.12 | 0.80 ± 0.11 |

Methods

Measures of BSAP, PICP, and NTX levels were determined for each patient through the course of therapy generally according to methods described above in Example 1.

Results

Data from this clinical trial including a total of 144 women treated with recombinant human parathyroid hormone (1-34), rhPTH(1-34) at 40 μg/day or treated with the antiresorptive alendronate (Fosamax®), and supplemented with vitamin D and calcium, for up to 18 months, showed results reported below.

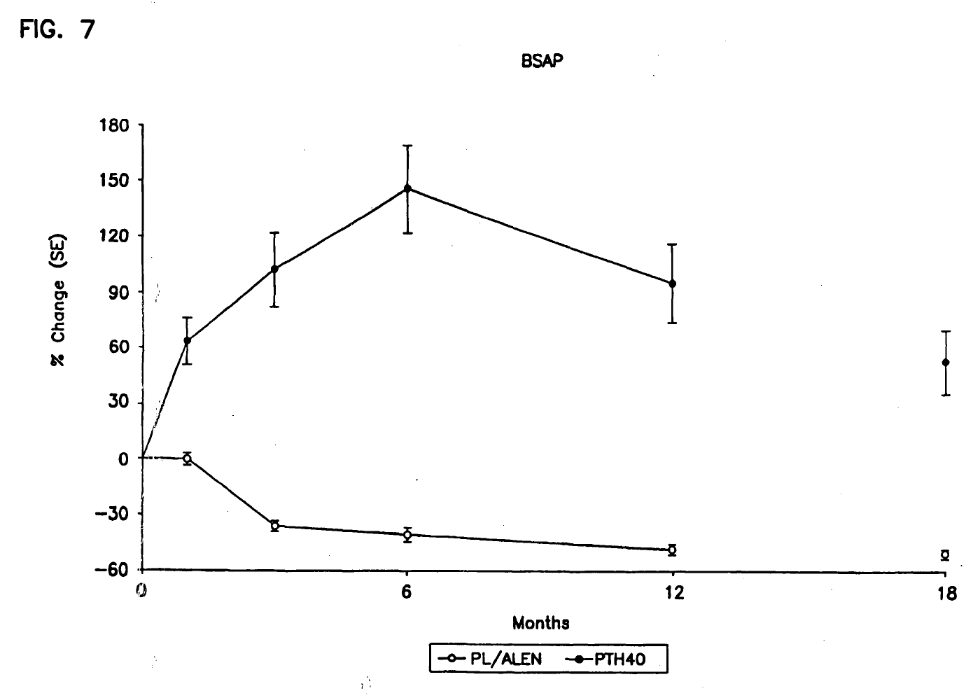

FIG. 7 illustrates daft showing the percent change (and standard error, SE) over time of BSAP serum levels in patients instead 40 μg/day PTH or alendronate. BSAP is a marker for bone formation, and thus increases in BSAP levels correlate with increases in bone formation A shown in FIG. 7, the present change in BSAP levels began to increase as early as one month and continued to increase reaching a peak at about 6 to about 12 months after initiation of PTH treatment in the 40 μg/day PTH population. At about 56 weeks following termination of PTH treatment (at 18 months from treatment initiation), BSAP remained at an elevated level. A decrease in BSAP level was observed in patients receiving alendronate after about 4 months (FIG. 7).

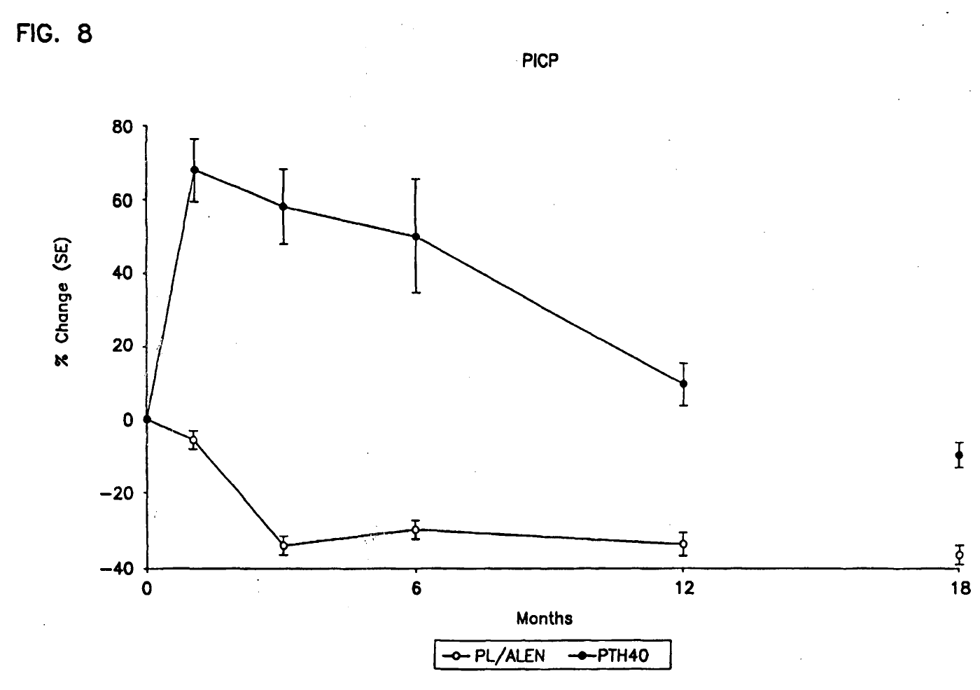

FIG. 8 illustrates data showing the percent change (and standard error, SE) over time of PICP serum levels in patients administered 40 μg/day PTH or alendronate. PICP is a marker for bone formation, and thus increases in PICP levels correlate with increases in bone formation. As shown in FIG. 8, the percent change in PICP levels increased rapidly and reached a peak within about one or two months after initiation of PTH treatment in the 40 μg/day PTH population. In contrast, a decrease in PICP was observed in patients receiving alendronate. After about 12 months of treatment, the PICP levels of PTH-treated patients approached control levels. At about 5-6 weeks following termination of PTH treatment (at 18 months from treatment initiation), PICP levels were the same pre-treatment levels, above alendronate-treated controls (FIG. 8).

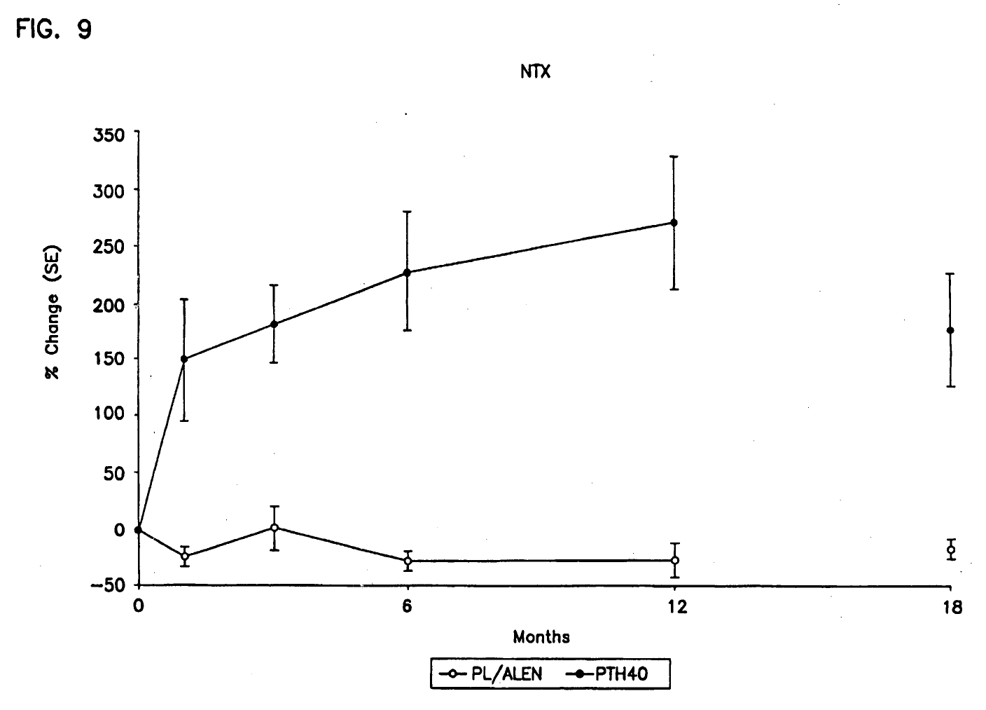

FIG. 9 illustrates data showing the percent change (and standard error, SE) over time of NTX urine levels in patients administered 40 μg/day PTH or alendronate. NTX is a marker for bone resorption, and thus increases in NTX levels correlate with increases in bone resorption. As shown in FIG. 9, the percent change in NTX levels began to increase in PTH treated subjects as early as one month into the study. In the 40 μg/day PTH population, the percent change in NTX levels increased steadily until about 12 months after treatment initiation. At about 5-6 weeks following termination of PTH treatment (at 18 months from treatment initiation), NTX levels had declined but remained elevated compared to pretreatment levels (FIG. 9). In alendronate treated group, NTX levels generally declined slightly during the first 6 months of the study and then remained diminished for the duration of the study (FIG. 9).

In summary, the data show that monitoring the selective regulation one or more of 3 markers, an enzyme indicative of osteoblastic processes of bone formation, BSAP, a product of collagen biosynthesis, PICP, and/or a product of collagen degradation, NTX, can be used to determine responders and duration of treatment with parathyroid hormone. Further, these data show that monitoring the selective regulation of one or more of 3 markers, an enzyme indicative of osteoblastic processes of bone formation, BSAP, a product of collagen biosynthesis, PICP, and/or a product of collagen degradation, NTX, can be used to distinguish administration of parathyroid hormone from administration of an antiresorptive.

Discussion

Based on the data presented above, monitoring one or more markers of bone turnover including an enzyme indicative of osteoblastic processes of bone formation, BSAP, a product of collagen biosynthesis, PICP, and/or a product of collagen degradation, NTX, can be used to establish efficacy of treatment, identify responders, and determine duration of treatment for a regimen including administration of PTH. This is in contrast to treatment with alendronate, which resulted in significantly different patterns in these markers. Thus, the method distinguished between therapy with an antiresorptive and with parathyroid hormone.

Changing profiles of bone markers can be used differentiate the effects of alendronate and/or to establish efficacy of treatment or to monitor actions of PTH and to determine duration of PTH therapy in patients whose skeletons are at risk of fracture. For example, early in treatment a rise in a product of collagen biosynthesis, PICP, no change in a product of collagen degradation, NTX, and/or some increase in an enzyme indicative of osteoblastic processes of bone formation, BSAP, can identify those patients that respond to PTH treatment. By way of fine example, a rise and maintained increase in an enzyme indicative of osteoblastic processes of bone formation, BSAP, normal level of a product of collagen biosynthesis, PICP, and/or normal or progressively increasing product of collagen degradation, NTX, over a period of months, can be used to confirm that patients continue to respond to PTH and that bone formation is active. Additionally, maintenance of elevated product of collagen degradation, NTX, after about 12-18 months, normal level of a product of collagen biosynthesis, PICP, and/or elevated enzyme indicative of osteoblastic processes of bone formation, BSAP can be used to signal that PTH therapy has run its course.

### EXAMPLE 4

### Biochemical Markers as Indicators of Bone Mineral Density Response to LY333334 (rhPTH(1-34)) in Women

Data from the studies described in Examples 1-3 above were further analyzed to develop more detailed models for the use of bone markers in monitoring and predicting effects of PTH on clinically significant correlates of efficacy in the treatment of osteoporosis, such as bone mineral density (BMD). Population pharmacodynamic (PD) models were developed to describe total lumbar spine and femoral neck bone mineral density (BMD) responses in ˜1500 postmenopausal women enrolled in a phase 3 study of LY333334 [rhPTH(1-34)]. Serum LY333334 (LY), procollagen 1 carboxy-terminal propeptide (PICP) and bone specific alkaline phosphatase (B SAP) concentrations, and urinary excretion of N-telopeptide (NTX) and free deoxypyridinoline (DPD) were also measured in a subset of ˜350 patients. LY dose, average steady-state LY concentration, and early changes in markers of bone turnover were each evaluated for their ability to predict subsequent changes in BMD. Overall, the PD model predicted a 10.5% and 2.9% increase in spine and femoral neck BMD, respectively, with LY 20 μg/day therapy for 21 months (actual increases from intent to treat analyses were 9.7% (spine) and 2.8% (femoral neck)). Response was greatest in patients with increased fracture risk (i.e., older women with low BMD, low body weight, and high bone turnover at baseline). In the subset analysis, PICP was the strongest indicator of BMD response; an increase >101 pM after 1 month of therapy was always associated with a gain in spine BMD. NTX was also a better predictor of increase in BMD than LY dose, but dose predicted BMD response better than LY, BSAP or DPD concentrations (p<0.001).

Methods Overview

Population pharmacodynamic models were developed individually for total lumber spine BMD, femoral neck BMD, procollagen 1 carboxy-terminal propeptide (PICP), bone specific alkaline phosphate (BSAP), urinary N-telopeptide (NTX), and urinary free deoxypyridinoline (DPD). These treatment-response models characterized change in the pharmacodynamic endpoints and identified significant patient factors influencing response to therapy.

The final treatment-response models for total lumbar spine and femoral neck BMD were used to calculate BMD values after 21 months of treatment for each patient, based on the individual's parameter estimates (empirical Bayesian estimates). Similarly, the final treatment-response models for each biochemical maker (PICP, BSAP, NTX, and DPD) were used to calculate concentration values after 1 month of treatment for each patient. These predicted BMD measurements were merged with the predicted biochemical marker concentrations for patients who completed at least 12 months of LY333334 therapy.

Biochemical marker response-indicator models were developed to characterize the relationship between the biochemical marker concentrations at 1 month and response to therapy, as measured by change in total lumbar spine and femoral neck BMD.

Results—Total Lumber Spine BMD

Patient Characteristics

The population pharmacodynamic evaluation of biochemical markers and total lumbar spine BMD included data from 276 postmenopausal women whose age ranged from 49 to 84 years at study entry and who weighed between 43.1 and 120 kg. Baseline measurements for spine BMD ranged from 0.38 to 1.31 g/cm². The range and mean values of age, weight and baseline spine BMD am shown in Table 1 (below).

TABLE 1
Demographics at Study Entry and Baseline
Spine Bone Mineral Density

| LY333334 | Age | Body Weight | Spine BMD |
|---|---|---|---|
| Treatment Group | (yr) | (kg) | (g/cm²) |
| 20-μg/day | | | |
| Range | 49-81 | 43.1-90.5 | 0.45-1.25 |
| Mean (% CV) | 68 (8.8%) | 65.2 (15.5%) | 0.81 (20.7%) |
| n^{a} | 143 | 143 | 143 |
| 40-μg/day | | | |
| Range | 50-84 | 45.0-120.0 | 0.38-1.31 |
| Mean (% CV) | 69 (10.1%) | 66.9 (17.7%) | 0.85 (20.3%) |
| n^{a} | 133 | 133 | 133 |

^{a}n = Number of patients included in the pharmacodynamic analysis.

The range and mean values for the biochemical markers at baseline are shown in Table 2 (below).

TABLE 2
Baseline Concentrations for Biochemical Markers

| LY333334 | | | | |
|---|---|---|---|---|
| Treatment | PICP | BSAP | NTX | DPD |
| Group | (pM) | (PM) | (nmBCE/L) | (nM) |
| 20-μg/day | | | | |
| Range | 52.0-255.0 | 2.0-43.6 | 7.7-143.2 | 2.2-16.1 |
| Mean | 116.7 (30.5%) | 12.5 (60.0%) | 48.2 (51.4%) | 7.1 (36.5%) |
| (% CV) | | | | |
| n^{a} | 143 | 143 | 143 | 143 |
| 40-μg/day | | | | |
| Range | 60.0-415.0 | 2.4-37.7 | 6.8-214.3 | 1.1-22.7 |
| Mean | 118.2 (34.0%) | 12.2 (58.1%) | 46.9 (61.7%) | 6.9 (41.0%) |
| (% CV) | | | | |
| n^{a} | 133 | 133 | 133 | 133 |

^{a}n = Number of patients included in the pharmacodynamic analysis.

Individual Predicted Biochemical Marker Concentrations and Change in Total Lumbar Spine BMD

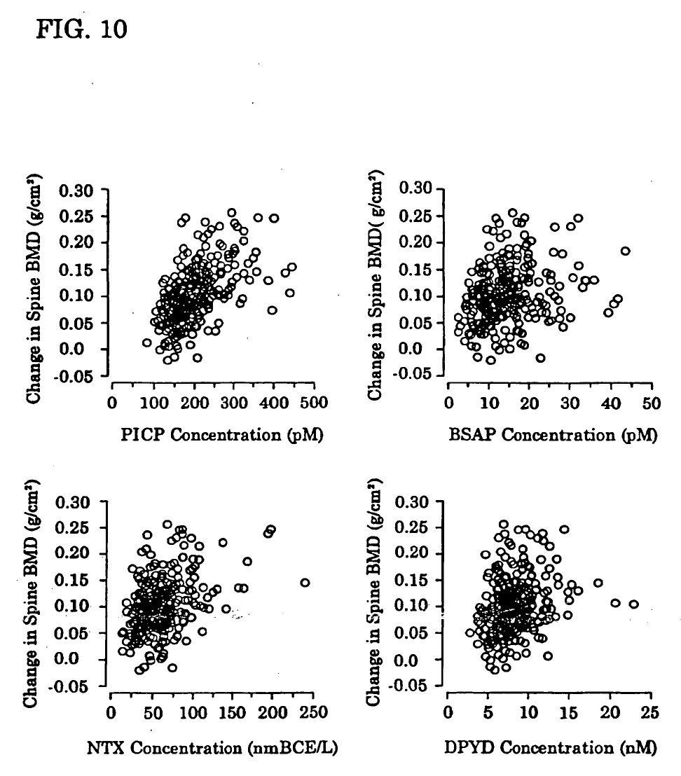

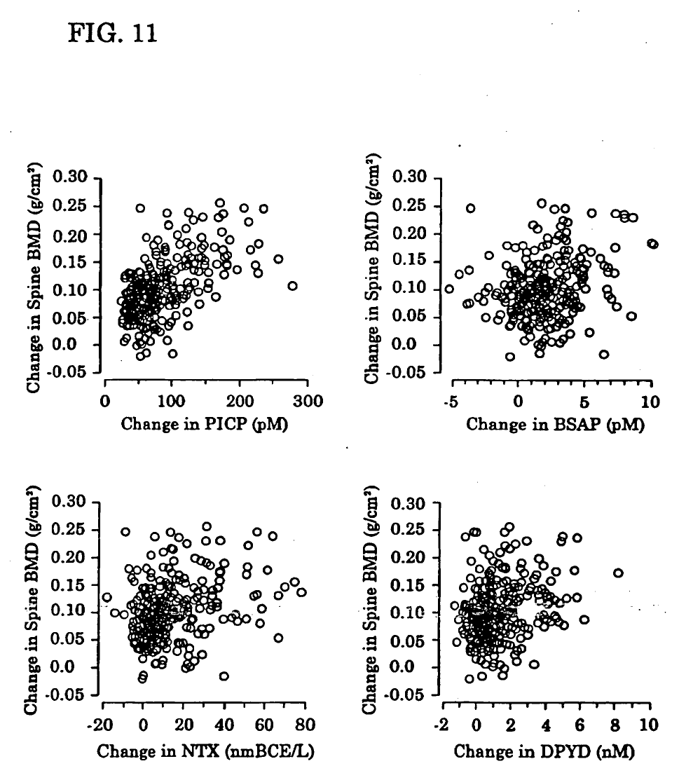

FIG. 10 illustrates the relationships between biochemical marker concentrations at 1 month and change in total lumbar spine BMD after 21 months of therapy. FIG. 11 shows the relationships between change from baseline for each biochemical marker at 1 month and change in total lumbar spine BMD after 21 months of therapy. Biochemical marker concentrations and spine BMD values are individual predictions from the final treatment-response model for each PD endpoint.

Individual Biochemical Markers Evaluations

A total of 276 individual predictions for spine BMD after 21 months of therapy were available for analysis. A base model was constructed which estimated the typical change in spine BMD after 21 months of LY333334 therapy and the associated inter-patient variability. This base model predicted a typical treated patient to have a 0.103 g/cm² (3.1% SEE) increase in spine BMD after 21 months. This corresponds to a 12.6% change from the mean baseline spine BMD of 0.82 gene. Inter-patient variability was estimated at 52.2% (9.1% SEE).

Treatment group was a significant predictor of change in spine BMD. The treatment group model predicted a change in spine BMD after 21 months of 0.086 g/cm² and 0.121 g/cm², respectively, for the 20-μg and 40-μg treatment groups. This corresponds to changes of 10.5% and 14.8% from the mean baseline spine BMD of 0.82 g/cm². Inter-patient variability was reduced to 48.6% (10.1% SEE).

Each biochemical marker was evaluated separately as an indicator of response to LY333334 treatment. The individual predicted biochemical marker concentrations at 1 month, as well as the resulting change from baseline, were tested as covariates on change in spine BMD after 21 months. Change in PICP from baseline was the strongest indicator of response to LY333334 therapy. Change in PICP at 1 month was a better predictor of change in spine BMD than LY333334 treatment group. The results of the individual biochemical marker evaluations are summarized in Table 3 (below).

TABLE 3
Individual Biochemical Marker Evaluations

| | Change In | Inter-Patient |
|---|---|---|
| Covariate | MOF | Variability |
| LY333334 Treatment Group | 46.818 | 48.6% (10.1% SEE) |
| Change in PICP at 1 Month | 103.322 | 44.8% (11.0% SEE) |
| NTX Concentration at 1 Month | 48.209 | 48.7% (9.7% SEE) |
| BSAP Concentration at 1 Month | 34.265 | 49.6% (9.4% SEE) |
| Change in DPD at 1 Month | 14.520 | 51.1% (9.6% SEE) |

Abbreviation: MOF = minimum value of objective function

Response-Indicator Model

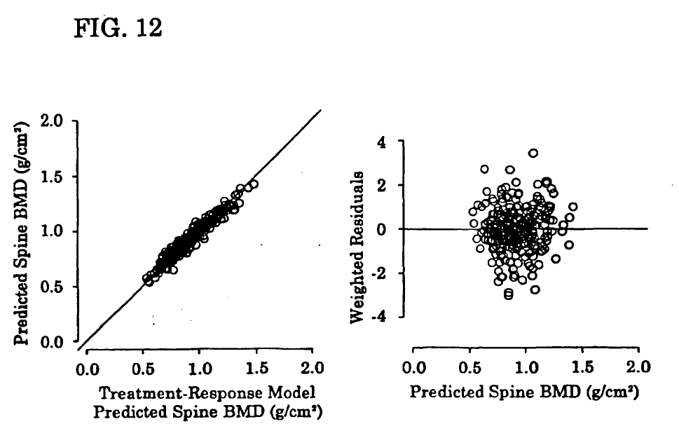

The individual biochemical marker evaluations were combined with patient factors identified in the final treatment-response model to produce the response-indicator model. The final response indicator model contained change in PICP at 1 month, BSAP concentration at 1 month, and age at study entry. Inclusion of these covariates decreased the between-patient variability to 42.5% (11.1% SEE). Goodness-of-fit of the final response indicator model is represented by agreement between predicted BMD values, as well as by weighted residuals (FIG. 12).

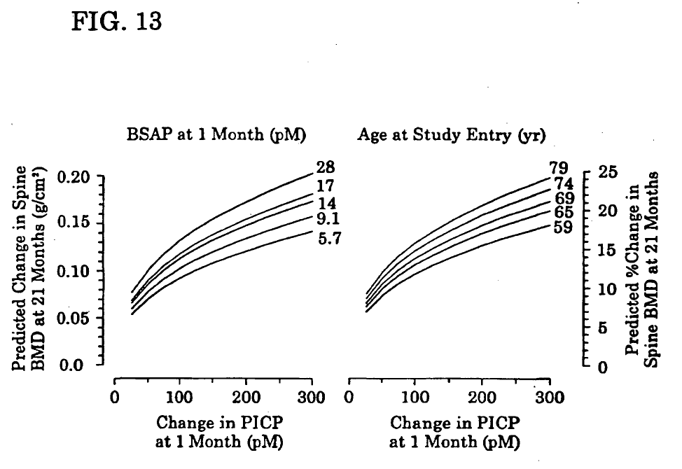

The predicted effect of each covariate on the change in spine BMD is described in Table 4 (below) and illustrated in FIG. 13. The model predicts a greater increase in spine BMD for patients with a larger change in PICP after 1 month of therapy. Patients with high BSAP concentrations at 1 month and older postmenopausal women were also predicted to have greater response to LY333334 therapy.

TABLE 4
Covariates in Final Response-Indicator Model,
Total Lumbar Spine Bone Mineral Density

| Covariate | Effect on Change in BMD | |
|---|---|---|
| Change in PICP at | Greater Increase | Greater increase |
| 1 Month | | in BMD |
| BSAP Concentration | Higher Concentration | [symbol] Greater increase |
| at 1 Month | | in BMD |
| Age at Study Entry | Older postmenopausal | [symbol] Greater increase |
| | women | in BMD |

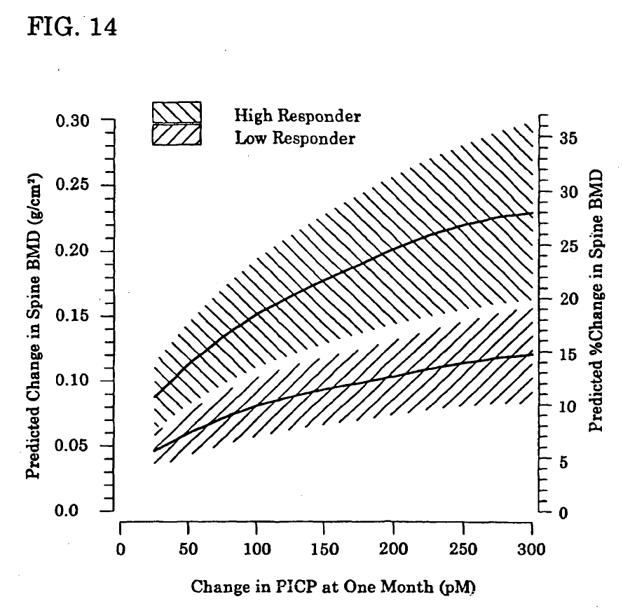

Change in PICP at 1 month and BSAP concentration at 1 month are both predicted to be indicators of response to LY333334 therapy. Age at study entry is also predicted to effect an individual patient's change in spine BMD. An older postmenopausal woman with high BSAP concentrations after 1 month of therapy would be predicted to have a greater increase in spine BMD for a given increase in PICP. A younger postmenopausal woman with low BSAP concentrations after 1 month would be predicted to have a lower increase in spine BMD. FIG. 14 shows the range of predicted response to LY333334 therapy for patients in these high and low responder categories.

Results—Femoral Neck BMD

Patient Characteristics

The population pharmacodynamic evaluation of biochemical markers and femoral neck BMD included data from 272 postmenopausal women whose age ranged from 49 to 84 years at study entry and who weighed between 45.0 and 120 kg. Baseline measurements for femoral neck BMD ranged from 0.40 to 0.88 g/cm². The range and mean values of age, weight and baseline femoral neck BMD are shown in Table 5 (below).

TABLE 5
Demographics at Study Entry and Baseline
Femoral Neck Bone Mineral Density

| LY333334 | Age | Body Weight | Spine BMD |
|---|---|---|---|
| Treatment Group | (yr) | (kg) | (g/cm²) |
| 20-μg/day | | | |
| Range | 49-81 | 45.6-90.5 | 0.40-0.88 |
| Mean (% CV) | 68 (8.8%) | 65.5 (15.1%) | 0.64 (15.1%) |
| n^{a} | 141 | 141 | 141 |
| 40-μg/day | | | |
| Range | 50-84 | 45.0-120.0 | 0.42-0.86 |
| Mean (% CV) | 69 (10.1%) | 66.9 (17.3%) | 0.65 (14.8%) |
| n^{a} | 131 | 131 | 131 |

^{a}n = Number of patients included in the pharmacodynamic analysis.

The range and mean values for the biochemical markers at baseline are shown in Table 6 (below).

TABLE 6
Baseline Concentrations for Biochemical Markers

| LY333334 | | | | |
|---|---|---|---|---|
| Treatment | PICP | BSAP | NTX | DPD |
| Group | (pM) | (pM) | (nmBCE/L) | (nM) |
| 20-μg/day | | | | |
| Range | 52.0-255.0 | 2.0-43.6 | 7.7-143.2 | 2.2-16.1 |
| Mean | 117.0 (30.5%) | 12.6 (59.4%) | 48.3 (51.5%) | 7.2 (36.4%) |
| (% CV) | | | | |
| n^{a} | 141 | 141 | 141 | 141 |
| 40-μg/day | | | | |
| Range | 60.0-415.0 | 2.4-37.7 | 6.8-214 | 1.1-22.7 |
| Mean | 118.1 (34.3%) | 12.1 (57.8%) | 47.3 (61.1%) | 6.9 (41.2%) |
| (% CV) | | | | |
| n^{a} | 131 | 131 | 131 | 131 |

^{a}n = Number of patients included in the pharmacodynamic analysis.

Individual Predicted Biochemical Marker Concentrations and Change in Femoral Neck BMD

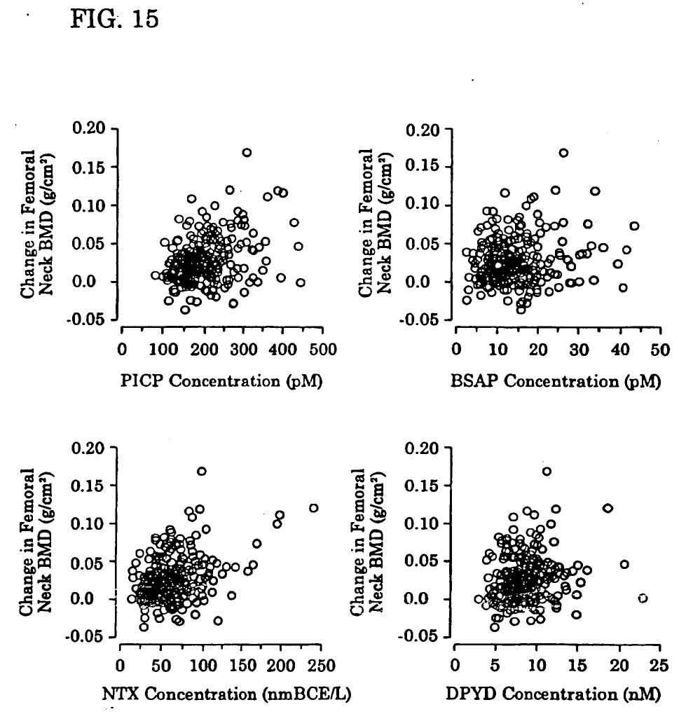

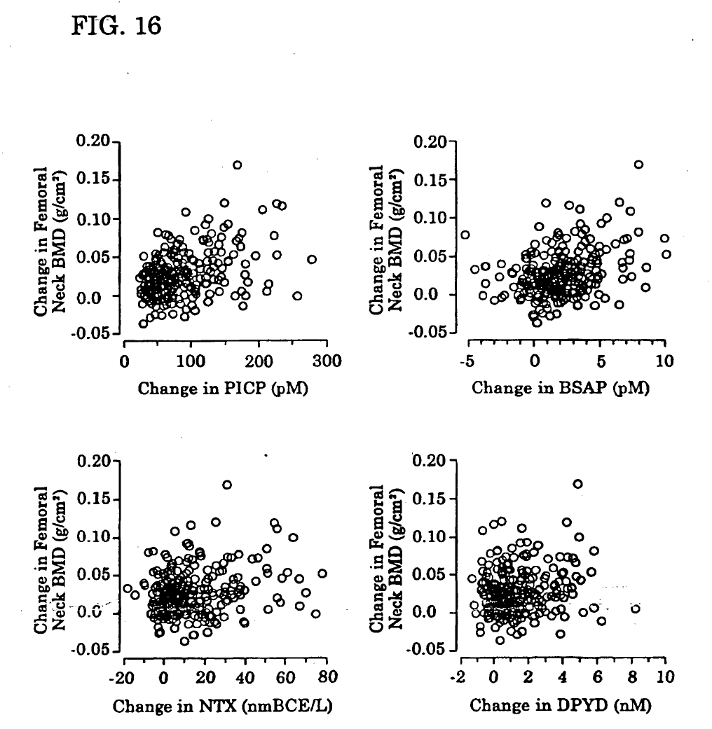

FIG. 15 illustrates the relationships between biochemical marker concentrations at 1 month and change in femoral neck BMD after 21 months of therapy. FIG. 16 shows the relationships between change from baseline for each biochemical marker at 1 month and change in femoral neck BMD after 21 months of therapy. Biochemical marker concentrations and femoral neck BMD values are individual predictions from the final treatment-response model for each PD endpoint.

Individual Biochemical Marker Evaluations

A total of 272 individual predictions for spine BMD after 21 months of therapy were available for analysis. A base model was constructed which estimated the typical change in femoral neck BMD after 21 months of LY333334 therapy and the associated inter-patient variability. This base model predicted a typical treated patient to have a 0.027 g/cm² (6.6% SEE) increase in femoral neck BMD after 21 months. This corresponds to a 4.2% change from the mean baseline BMD value of 0.64 g/cm². Inter-patient variability was estimated at 109.5% (12.5% SEE).

Treatment group was a significant predictor of change in femoral neck BMD. The treatment group model predicted a change in femoral neck BMD after 21 months of 0.018 g/cm² and 0.034 g/cm², respectively, for the 20-μg and 40-μg treatment groups. This corresponds to changes of 2.8% and 5.3% from the mean baseline BMD value of 0.64 g/cm². Inter-patient variability was reduced to 103.4% (14.1% SEE).

Each biochemical marker was evaluated separately as an indicator of response to LY333334 treatment. The individual predicted biochemical marker concentrations at 1 month, as well as the resulting change from baseline, were tested as covariates on change in femoral neck-BMD after 21 months. Change in PICP from baseline was the strongest indicator of response to LY333334 therapy. Change in PICP at 1 month was a better predictor of change in femoral neck BMD than LY333334 treatment group. The results of the individual biochemical marker evaluations are summarized in Table 7 (below).

TABLE 7
Individual Biochemical Marker Evaluations

| | Change | Inter-Patient |
|---|---|---|
| Covariate | in MOF | Variability |
| LY333334 Treatment Group | 73.873 | 103.4% (14.1% SEE) |
| Change in PICP at 1 Month | 82.054 | 103.0% (14.0% SEE) |
| NTX Concentration at 1 Month | 55.671 | 104.4% (12.8% SEE) |
| Change in BSAP at 1 Month | 38.200 | 106.8% (12.7% SEE) |
| DPD Concentration at 1 Month | 12.598 | 109.1% (12.5% SEE) |

Abbreviation: MOF = minimum value of objective function

Biochemical Marker Response Indicator Model

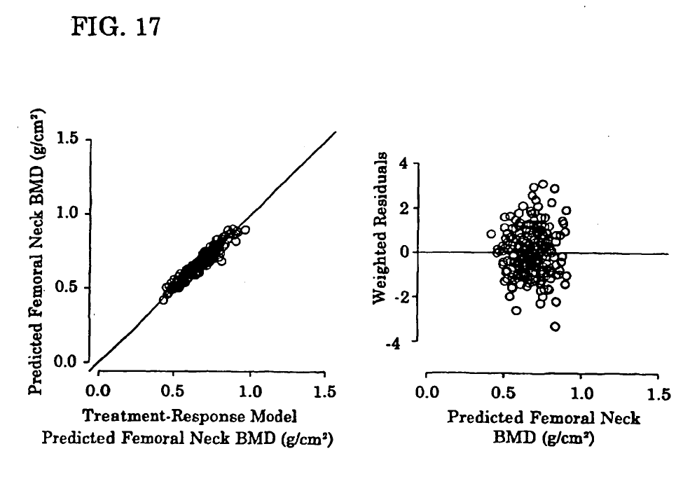

The individual biochemical marker evaluations were combined with patient factors identified in the final treatment-response model to produce the response-indicator model. The final response indicator model contained only change in PICP at 1 month. Inclusion of this covariates decreased the between-patient variability to 103.0% (14.0% SEE). Goodness-of-fit of the final response indicator model is represented by agreement between predicted BMD values, as well as by weighted residuals (FIG. 17).

The predicted effect of this covariate on the change in spine BMD is described in Table 8 (below). The model predicts a greater increase in spine BMD for patients with a larger change in PICP after 1 month of therapy.

TABLE 8
Covariates in Final Response-Indicator Model,
Femoral Neck Bone Mineral Density

| Covariate | Effect on Change in BMD | |
|---|---|---|
| Change in PICP | Greater Increase | Greater increase in BMD |
| at 1 Month | | |

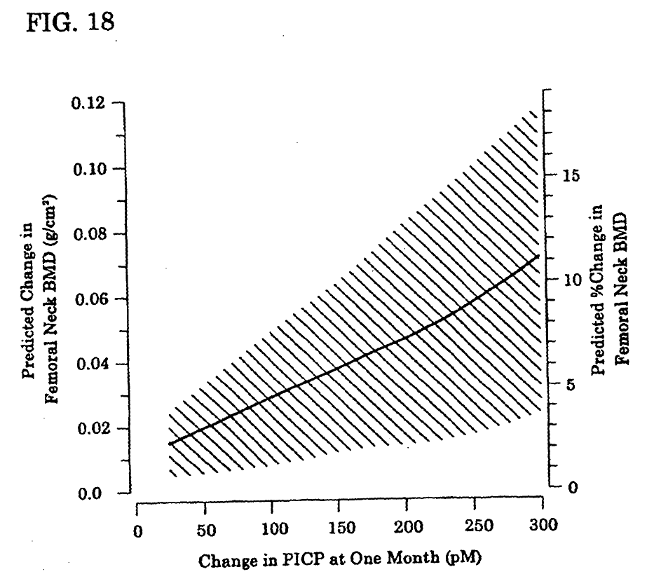

FIG. 18 shows the range of predicted response to LY333334 therapy from the final response-indicator model.

Discussion

This example provides pharmacodynamic analyses of the changes in bone mineral density and biochemical markers of bone formation and resorption, in response to LY333334 treatment, are also reported. The pharmacodynamic responses to LY333334 treatment were evaluated by population methods of analysis from data obtained in a setting that resembles clinical practice. Additional benefits of the population analyses include the ability to characterize the intra- and inter-subject variability in the pharmacodynamic parameters as well as patient factors (such as demographics and laboratory values) that could influence the disposition or response to the compound.

Population pharmacodynamic analyses were undertaken to evaluate the time course of the relationships between efficacy measures and LY333334 dose or LY333334 concentrations. The results of the GHAC efficacy trial (disclosed in PCT Patent Application No. PCT/US99/18961) showed that LY333334 treatment of postmenopausal osteoporotic women significantly increased bone mineral density in both the spine and hip regions and, furthermore, reduced the incidence of new vertebral fractures and non-vertebral fractures, compared to placebo. The population pharmacodynamic analyses of total lumbar spine and hip (femoral neck) BMD for patients receiving 20 or 40 μg/day LY333334 also showed increase in BMD over time. As a part of this assessment, a population placebo-response model describing the change in BMD in patients randomly assigned to placebo (supplemented with calcium and vitamin D) was first developed. Patient-specific factors that explained some of the variability of that model were identified and included in the model. A pharmacodynamic model describing the therapeutic response was then developed for patients randomly assigned to LY333334 treatment using the placebo-response model as the baseline function. Thus, the progression of bone loss that occurs in osteoporosis patients receiving only calcium and vitamin D supplementation was separated from the effects of LY333334 treatment.

The time course of biochemical marker response to LY333334 dose was extensively evaluated as part of the overall population pharmacodynamic analyses. Pharmacodynamic models were developed for four biochemical markers: PICP and BSAP (biochemical measures of bone formation); NTX and five deoxypyridinoline (biochemical measures of bone resorption). Patient-specific factors that explained some of the variability of each model were identified and included in the model. As an additional evaluation, the relationship between LY333334 exposure and PICP response was modeled. Finally, the biochemical markers were evaluated as potential indicators of response to therapy by modeling the relationship between a change in the biochemical endpoint after 1 month of treatment and the increase in spine and femoral neck BMD after 21 months of treatment. The final response-indicator model suggested that the increase in PICP after 1 month of treatment, relative to the baseline PICP concentration, was more accurate than either LY333334 dose or concentration in predicting the BMD response at 21 months. Additional patient-specific factors were identified, which further decreased the variability in this predictive model.

Total Lumbar Spine Bone Mineral Density

The population pharmacodynamic evaluation of total lumbar spine BMD included data from 1516 patients randomly assigned to receive LY333334 40 μg/day (n=504), LY333334 20 μg/day (n=502), or placebo (n=510). The placebo-response model demonstrated an insignificant increase in total lumbar spine BMD for the typical patient receiving placebo treatment (plus calcium and vitamin D supplementation). This suggests that patients who were randomly assigned to placebo treatment benefited from calcium and vitamin D supplementation since bone loss would have been expected over an 18 to 24-month period in this patient population. Nevertheless, the rate of change in total lumbar spine BMD varied between the patients. Younger women with osteoporosis simply maintained bone density in the spine, whereas the older patients actually increased bone density in the spine, as much as 3% for a patient who began therapy at 80 years of age.

Bone loss due to decease in estrogen production is the major cause of osteoporosis in postmenopausal women. Women lose bone more rapidly early after menopause, and the rate of bone loss tends to slow with advancing age. It has also been reported that women who are underweight have a higher risk for osteoporosis. Body weight, however, did not appear to influence the rate of change in total lumbar spine BMD in the placebo-treated patients. Nevertheless, dietary supplements of calcium and vitamin D are thought to contribute to the maintenance of total number spine BMD. Results from the current analysis clearly support these observations.

LY333334 increases both bone formation and resorption, thereby increasing the overall rate of bone turnover. The net effect is a significant increase in bone mineral density. The time course of change in total lumbar spine BMD for the LY333334-treated groups is best described by a curvilinear relationship. The population-predicted time course suggests that the rate of increase in BMD is greatest during the first year of treatment.

Bone status at baseline, as reflected by total lumbar spine BMD or by NTX, was a significant predictor of response to LY333334 therapy. Those patients having lower initial BMD and/or higher initial NTX concentrations were shown to have the greatest increase in total lumbar spine BMD. Age remained a significant predictor of response to therapy (retained from the placebo-response model) such that the therapeutic effect of LY333334 was greatest in older patients. Of note, the number of years since menopause did not effect the magnitude of the response to LY333334 treatment. Furthermore, although age and baseline BMD status were both found to influence the magnitude of the response to LY333334 therapy, the two covariates were not correlated.

Each of the three covariates shown to influence response to LY333334 treatment (increased age at study entry, increased baseline NTX excretion, and decreased spine BMD) are indicative of high bone turnover states, and therefore, an expanded pool of osteoblasts. Presumably, LY333334 acts upon the pool of osteoblasts to cause bone formation to exceed bone resorption, thereby increasing bone mass. Patients with an enhanced pool of available osteoblasts at study entry, are therefore, more responsive to LY333334 therapy. The pharmacodynamic model suggests that an older patient beginning therapy in an existing state of high bone turnover would have an increase in total lumbar spine BMD that is twice the amount achieved in a younger patient with low bone turnover status.

In order to explore the relationship between concentration and the effect on spine BMD, a pharmacokinetic/pharmacodynamic model was developed. This relationship was best described by a sigmoid Eₘₐₓ model with AUC₅₀ estimated at 170 pg·hr/mL. The post-hoc estimates of AUC from the pharmacokinetic model suggest that systemic exposure from the 20 μg dose (average AUC, 365 pg·hr/mL) and 40 μg dose (average AUC, 576 pg·hr/mL) produce an increase in spine BMD that is 82% and 92% of the maximum effect, respectively. While the Emax model improved the ability of the pharmacodynamic model to predict the increase in spine BMD after 21 months of therapy, the actual administered dose proved to be a better indicator of response. Thus, the final pharmacodynamic model which included treatment group rather than systemic exposure, predicted the increase in spine BMD in a patient of average age (˜69 years), baseline spine BMD (˜0.82 g/cm²), and baseline NTX concentration (˜48 nmBCE/L) to be approximately 10.5% and 14.6% after 21 months of 20 μg/day and 40 μg/day therapy, respectively.

Hip (Femoral Neck) Bone Mineral Density

The population pharmacodynamic evaluation of hip (femoral neck) BMD included data from 1466 patients randomly assigned to receive LY333334 40 μg/day (n=491), LY333334 20 μg/day (n 488), or placebo (n=487). The placebo response model indicated that an insignificant amount of bone density was lost during the treatment period but that the rate of bone loss was influenced by body weight. Patients with low body weight lost as much as 2.5% of their baseline femoral neck BMD.

The LY333334 treatment-response model indicated that LY333334 increased femoral neck BMD over the treatment period. The time course of change in femoral neck BMD for the LY333334-treated groups is best described by a linear relationship. As with total lumbar spine BMD, age and bone turnover status at study entry were significant predictors of change in femoral neck BMD. Body weight also remained a significant predictor of response to therapy (retained from the placebo-response model). Therefore, the therapeutic effect of LY333334 was greatest in older patients with low body weight and high urinary NTX excretion, i.e., high bone turnover, indicative of enhanced osteoblast availability at study entry. The pharmacodynamic model suggests that an older patient beginning therapy in an existing state of high bone turnover would have an increase in femoral neck BMD that is nearly seven times the amount achieved in a younger patient with low bone turnover status. Despite the identification of these patient factors which influenced change in femoral neck BMD response, the magnitude of the inter-patient in the final pharmacodynamic model was high, suggesting that additional, unidentified factors may also contribute to variability in response.

A pharmacokinetic/pharmacodynamic model was also developed for femoral neck BMD. The relationship was best described by a sigmoid Eₘₐₓ model with AUC₅₀ estimated at 283 pg·hr/mL. The higher AUC₅₀ for the hip BMD model suggests that greater LY333334 systemic exposure is required to reach a maximum response at the hip. Nevertheless, post-hoc estimates of AUC from the pharmacokinetic model suggest that systemic exposure from the 20 μg dose (average AUC, 365 pg·hr/mL) and 40 μg dose (average AUC, 576 pg·hr/mL) produce an increase in femoral neck BMD that is 56% and 67% of the maximum effect, respectively. While the Eₘₐₓ model improved the ability of the pharmacodynamic model to predict the increase in hip BMD after 21 months of therapy, the actual administered dose proved to be a better indicator of response. Thus, the final pharmacodynamic model which included treatment group rather than systemic exposure, predicted the increase in hip BMD in a patient of average age (˜69 years), body weight (˜66 kg), and baseline NTX concentration (˜48 mmBCE/L) to be approximately 2.8% and 5.2% after 21 months of 20 μg/day and 40 μg/day therapy, respectively.

Pharmacodynamics of Biochemical Markers of Bone Formation and Resorption

An extensive investigation was undertaken to evaluate the time course of biochemical markers of bone formation (PICP and BSAP) and resorption (NTX and DPD) during LY333334 therapy. The population pharmacodynamic evaluation of these biochemical markers included data from approximately 340 patients randomly assigned to receive LY333334 40 μg/day (n≅170) or LY333334 20 μg/day (n≅170). The biochemical markers did not appear to change from baseline during placebo treatment, therefore, patients assigned to receive placebo were not included in these datasets. Pharmacodynamic models based on linear, exponential, and spline functions of time were evaluated. With the exception of an initial elevation followed by an exponential decline function for PICP, spline models proved to best fit the data for the remaining three biochemical markers. These models reflect the complex time course of change in the underlying processes of bone formation and bone resorption occurring throughout the skeleton in response to the anabolic action induced by LY333334.

Biochemical Markers of Bone Formation

PICP increased rapidly, reaching a maximum at or before the first observation at 1 month, and then declined in an exponential fashion. The time course of BSAP response was slower, with BSAP concentrations demonstrating a peak response 6 months after initiation of treatment. This response was maintained even at 12 months, the last observation while patients were still on therapy. The time course for the biochemical markers of bone formation is consistent with the known anabolic effect of LY333334: PICP, a measure of collagen formation, responds more rapidly than BSAP, which is a measure of bone mineralization.

As with total lumbar spine BMD, the baseline value of each biochemical marker of bone formation served as a predictor of its own overall rate of change. Inclusion of the baseline parameter as a covariate accounted for a significant portion of between-patient variability in the final population model. Patients with high baseline values of BSAP, indicative of high bone turnover, experienced a greater increase in BSAP response. Baseline BSAP may reflect the number of osteoblasts at the onset of LY333334 treatment. Thus, a larger number of osteoblasts available at that the onset of therapy may expand the pool of osteoblasts to a greater extent than if that pool were smaller to begin with.

LY333334 had a nearly dose proportional effect on the magnitude of the response for both biochemical markers. The response in the 40 μg/day treatment group was 94% and 73% greater than the 20 μg/day treatment group for PICP and BSAP endpoints, respectively. Additionally, patients with larger increases in PICP concentrations were those with a lower body mass index and non-smokers. Although unproven, it is possible that the higher peak LY333334 concentrations, observed in patients with decreased body weight, are responsible for the more dramatic PICP response. In addition, smokers have lower estrogen concentrations, which may have diminished the response of osteoblast activity to LY333334. Insufficient data on baseline estrogen concentrations in this subset of patients did not allow for estrogen to be assessed as a potential covariate for either bone marker or BMD response. Patients with higher baseline concentrations of 1,25 dihydroxyvitamin D (a calcium-regulating hormone) demonstrated a slower rate of decline as PICP concentrations returned to baseline, than did patients with lower baseline concentrations. This observation may be related to the well-established dependency of PTH action on vitamin D status. Variability in the treatment-response models remained high, even with the identification of these covariates, suggesting that additional, unidentified factors may also contribute to variability in response.

A pharmacokinetic/pharmacodynamic model was developed for the PICP response. As with BMD, this relationship was best described by a sigmoid Eₘₐₓ model with AUC₅₀ estimated at 239 pg·hr/mL Post-hoc estimates of AUC from the pharmacokinetic model suggest that systemic exposure from the 20 μg dose (average AUC, 365 pg·hr/mL) and 40 μg dose (average AUC, 576 pg·hr/mL) produce an increase in PICP concentration at 1 month that is 70% and 85% of the maximum effect, respectively. While the Eₘₐₓ model improved the ability of the pharmacodynamic model to predict the increase in PICP concentration after 1 month of therapy, the actual administered dose proved to be a better indicator of response. Thus, LY333334 exposure was a less significant predictor of elevation in PICP concentrations than administered dose.

Biochemical Markers of Bone Resorption

In general, the time course of response for biochemical markers of bone resorption was slower than the response for the markers of formation. This is not unexpected for an anabolic agent and suggests that LY333334 stimulates bone formation first, followed by bone resorption. Peak urinary NTX excretion occurred at the last observation while on therapy, that is at 12 months. Urinary DPD concentrations peaked 6 months after initiation of treatment. This response was maintained even at 12 months, the last observation while patients were still on therapy.

A near dose proportional effect of the magnitude of response was also observed for the biochemical markers of bone resorption. The response in the 40 μg/day treatment pup was 87% and 83% greater than the 20 μg/day treatment group for NTX and DPD endpoints, respectively.

A interesting finding in the covariate analysis is that higher baseline concentrations of endogenous PTH were associated with a progressive decline in NTX excretion as a function of LY333334 therapy. This may reflect the fact that higher sustained concentrations of endogenous PTH could potentially down-regulate osteoblast receptors and desensitize those cells to the effects of the short-term exposure to exogenous PTH(1-34) concentrations achieved when LY333334 is administered.

Of note, high baseline concentrations of biochemical marker of bone formation and low baseline femoral neck BMD were associated with greater responses to LY333334 therapy for both NTX and DPD. Both types of covariances reflect increased bone turnover and increased numbers of osteoblasts. One explanation for the effect of bone formation markers on bone resorption activity is the requirement of the osteoblast to maintain osteoclastic bone resorption. That is, the enhanced pool of osteoblasts may stimulate osteoclastic bone resorption which, in turn, augments the LY333334 stimulation of bone resorption, as measured by increased NTX and/or DPD excretion. In any case, the results of the covariate analysis for biochemical markers of bone formation and resorption suggest that the anabolic effect of LY333334 is enhanced in patients who already have a high rate of bone turnover at initiation of LY333334 therapy. Nevertheless, variability in the treatment-response models remained high, even with the identification of these covariates, suggesting that additional, unidentified factors may also contribute to variability in response.

Biochemical Markers as Indicators of Bone Mineral Density Response to LY333334 Treatment in Postmenopausal Women

Biochemical-response indicator models were developed to characterize the relationship between biochemical marker concentrations at 1 month and response to therapy, as measured by change in total lumbar spine and hip (femoral neck) BMD. The objective of this analysis was to determine if the magnitude of the change in biochemical markers was an early indicator of the eventual change in total lumbar spine and femoral neck BMD after 21 months of treatment.

The magnitude of the change in PICP concentration at 1 month was shown to be a better predictor of the change in total lumbar spine or femoral neck BMD at 21 months than other biochemical markers. Furthermore, PICP was a better predictor of BMD response than dose, which predicted the magnitude of BMD response for the 2-μg/day and 40-μg/day treatment groups.

The change from baseline in PICP concentration at 1 month was more effective in predicting BMD outcome for total lumbar spine than for femoral neck. Variability in the response-indicator model for spine was further reduced by the inclusion of age and BSAP concentration (1 month after initiation of therapy) as covariates. For a given increase in PICP concentration at 1 month, relative to baseline, older patients and/or patients with a high BSAP concentration at 1 month are predicted to have a greater increase in total lumbar spine BMD after 21 months of therapy than younger patients and/or patients with a low BSAP concentration at 1 month.

While the biochemical-response indicator models cannot be used as presently developed to definitively predict which patients will or will not respond to LY333334 therapy, some useful correlations between bone markers and BMD are readily apparent. For instance, an increase in PICP concentration above baseline of at least about 101 pM, about 1 month after initiation of therapy, was clearly associated with a robust improvement in total lumbar spine BMD in all patients included in this analysis. Further, as seen in FIG. 11, only four subjects (from a total of 272) analyzed in the present study showed a negative BMD response (spine BMD<0.00 g/cm²). One of these slightly negative responders had a PICP level of about 100 pM, while the other three had PICP levels less than about 70 pM but above about 50 pM. Accordingly, only one of 272 subjects with a PCIP value above about 70 pM, and three, above about 50 pM, had a negative BMD response. In addition, about nine subjects with minimal positive BMD response (≦ about 0.02 g/cm²) also had PICP levels less than about 100 pM, with four of these at or below 50 pM. Finally, the minimum increase in PICP level in the entire study population was at least about 20 pM. Therefore, only four of 272 subjects with a PICP value above about 20 pM had a negative BMD response, and only about thirteen with such a PICP value had a BMD response below about 0.02 g/cm².

Accordingly, while inter-patient variability in femoral neck BMD response is too high to 100% accurately distinguish responders from non-responders based solely upon a change in PICP concentration at 1 month, PICP increment values at about 1 month of PTH treatment of at least about 20 pM, preferably at least about 50, and more preferably at least about 100 pM are associated with increasing probabilities of a strong BMD response indicative of significant clinical efficacy in the treatment of osteoporosis. Moreover, analyses of patterns of bone marker levels, including PICP and other bone markers described above, along with patient characteristics such as base level BMD, age and base level body weight, provides further guidance on treatment with PTH which is needed, for instance, to avoid or change ineffective dosing as soon as possible after initiation of treatment, and to terminate treatment after optimum clinical benefits are achieved.

Conclusions on Pharmacodynamic Responses to LY333334 Treatment in Women:

Placebo-response model (calcium and vitamin D supplementation)
The mean change in total lumbar spine and hip (femoral neck) bone mineral density (BMD) was insignificant over the observed treatment period (median duration of treatment, 21 months) in placebo-treated patients who were supplemented with calcium and vitamin D; nevertheless, the change varied between patients.
Older women with osteoporosis gained up to 3% total lumbar spine BMD whereas younger osteoporotic patients maintained bone density in the spine.
The rate of femoral neck BMD loss is greater in patients with low body weight.

LY333334 treatment-response model
Total lumbar spine BMD increases 10.5% and 14.6% with LY333334 20 μg/day and 40 μg/day treatment, respectively.
Hip (femoral neck) BMD increases 2.8% and 5.2% with LY333334 20 μg/day and 40 μg/day treatment, respectively.
Older women with osteoporosis had greater improvement in total lumbar spine BMD than younger women. Bone status (low spine BMD and/or high urinary N-telopeptide [NTX] concentration) at initiation of LY333334 treatment is also correlated with greater spine BMD response to LY333334.
Advanced age, increased body weight, and high NTX concentration at baseline were associated with greater femoral neck BMD response to LY333334.
   Biochemical markers of bone formation and resorption
Biochemical markers of bone formation (serum procollagen I carboxy-terminal propeptide [PICP] and bone-specific alkaline phosphatase [BSAP]) responded more rapidly to LY333334 treatment than did biochemical markers of bone resorption (NTX and DPD). Nevertheless, both sets of markers were sensitive measure of acute changes in bone metabolism.
A near dose proportional effect of the magnitude of response was observed for all biochemical markers of bone formation and resorption.
In general, higher baseline concentrations of biochemical markers of bone formation (indicative of increased bone turnover) were associated with a greater response to LY333334 treatment for all biochemical markers.
The increase in PICP concentration 1 month after initiation of therapy, is a better predictor than dose, of the ultimate increase in total lumbar spine and femoral neck BMD after 21 months of therapy. While the correlation of change in PICP at 1 month to change in spine BMD at 21 months cannot be used to definitively predict which patients will or will not respond to LY333334 therapy, an increase in PICP concentration of at least 101 pM was associated with a robust improvement in total lumbar spine BMD in all patients.

### EXAMPLE 5

### Increased Bone Density Upon Administration of rhPTH(1-34) to Human Males with Osteoporosis

Objectives: The primary objective of this study was to demonstrate an increase in vertebral BMD in men with pry osteoporosis following 2-year treatment with LY333334 (rhPTH(1-34)) 40 μg/day plus calcium and vitamin D or LY333334 20 μg/day plus calcium and vitamin D, compared with patients treated with calcium and vitamin D alone.
Methodology: This stay was a double-blind, calcium- and vitamin D-controlled, parallel, randomized study. Four hundred thirty seven men with primary osteoporosis were enrolled in the study. Approximately one-third of die patients were randomly assigned to LY333334 40 μg/day plus calcium and vitamin D, one-third of the patients were randomly assigned to LY333334 20 μg/day plus calcium and vitamin D, and one-third of the patients were randomly assigned to placebo plus calcium and vitamin D.
Number of Subjects:
   PTH: Male 437, Female 0, Total 437;
   LY333334 20 μg: Male: Total 151.
   LY333334 40 μg: Male: Total 139.
   Placebo: Male: Total 147.
Diagnosis and Inclusion Criteria: The study patients were men with primary osteoporosis, aged 30 to 85 years, inclusive. L-2 to L-4 vertebrae must have been intact without artifacts, crush fracture or other abnormalities which would have interfered with the analysis of the posterior-anterior lumbar spine bone mineral density (BMD) measurement which must have been at least 2.0 SD below that of young, healthy men.
   Dosage and Administration:
   Test Product
   LY333334: 20-μg/day, given once daily; 40-μg/day, given once daily Placebo, given once daily.
   Reference Therapy
   Calcium tablets 1000 mg/day, given once daily:
   Vitamin D tablets 400 IU given once daily
Duration of Treatment:
   LY333334:
   20-μg group: 297.5 days
   40-μg group: 282.6 days
   Placebo: 312.92 days
Criteria for Evaluation:
   The primary objective of this study was to demonstrate an increase in vertebral BMD in men with primary osteoporosis following 2-year treatment with LY333334 40 μg/day plus calcium and vitamin D or LY333334 20 μg/day plus calcium and vitamin D, compared with patients treated with calcium and vitamin D alone.
   An efficacious response was defined as a statistically significant difference in the change in vertebral BMD of the group receiving LY333334 compared with the group receiving placebo.
      Patient Demographic and Other Baseline Characteristics

The demographic characteristics (racial origin, age, height, weight and BMI) of the patients at study entry were not statistically significantly different among the three treatment groups at baseline (Table 9, below). The mean age at study entry was 58.68 years. Most of the patients were Caucasian (99.1%). The mean BMI at baseline was 25.15 kg/m².

The treatment groups were comparable at baseline with respect to smoking habits and alcohol and caffeine consumption. Of the 437 randomly assigned patients, 29.7% were smokers, 70% consumed more than 3 drinks daily, and 87.9% consumed caffeine.

No significant differences among treatment groups were observed in consumption of dietary calcium or any previous osteoporotic drug use at baseline. Treatment groups were comparable at baseline with respect to type of osteoporosis (51% idiopathic, 49% hypogonadal), previous nonvertebral fractures, and baseline vertebral BMD. Of the 437 randomly assigned-patients, 59% had a prevalent nonvertebral fracture and the mean baseline vertebral BMD was 0.87 g/cm².

TABLE 9
Patient Demographics and Baseline Characteristics-All Randomly Assigned Patients

| | Placebo | | PTH20 | | PTH40 | | Total | | |
|---|---|---|---|---|---|---|---|---|---|
| Characteristic | (N = 147) | | (N = 151) | | (N = 139) | | (N = 437) | | P-Value |
| Age (years) | 58.65 ± 12.87 | | 59.29 ± 13.40 | | 58.06 ± 12.68 | | 58.68 ± 12.98 | | 0.724 |
| (mean ± SD) | | | | | | | | | |
| Origin n (%) | | | | | | | | | 0.725 |
| Caucasian | 147 | (100) | 149 | (98.7) | 137 | (98.6) | 433 | (99.1) | |
| Asian | 0 | | 1 | (0.7) | 1 | (0.7) | 2 | (0.5) | |
| Other | 0 | | 1 | (0.7) | 1 | (0.7) | 2 | (0.5) | |
| Body mass index | 25.21 ± 3.61 | | 25.37 ± 3.72 | | 24.86 ± 3.60 | | 25.15 ± 3.64 | | 0.483 |
| (kg/m²) (mean ± | | | | | | | | | |
| SD)^{a} | | | | | | | | | |
| Height (cm) | 173.63 ± 7.40 | | 173.72 ± 7.34 | | 172.99 ± 7.45 | | 173.46 ± 7.39 | | 0.665 |
| (mean ± SD)^{b} | | | | | | | | | |
| Weight (kg) | 75.98 ± 11.54 | | 76.59 ± 12.25 | | 74.47 ± 12.16 | | 75.71 ± 11.99 | | 0.305 |
| (mean ± SD) | | | | | | | | | |
| Current smoker n | 47 | (32.0) | 45 | (29.8) | 38 | (27.3) | 130 | (29.7) | 0.693 |
| (% yes) | | | | | | | | | |
| Alcohol n (% yes) | 102 | (69.4) | 114 | (75.5) | 90 | (64.7) | 306 | (70.0) | 0.134 |
| Caffeine n (% | 130 | (88.4) | 128 | (84.8) | 126 | (90.6) | 384 | (87.9) | 0.425 |
| yes) | | | | | | | | | |
| Previous | 17 | (11.6) | 22 | (14.6) | 25 | (18.0) | 64 | (14.6) | 0.308 |
| osteoporosis | | | | | | | | | |
| drug user n (% | | | | | | | | | |
| yes) | | | | | | | | | |
| Osteoporosis type | | | | | | | | | 0.974 |
| n (%) | | | | | | | | | |
| Idiopathic | 74 | (50.3) | 78 | (51.7) | 71 | (51.1) | 223 | (51.0) | |
| Hypogonadal | 73 | (49.7) | 73 | (48.3) | 68 | (48.9) | 214 | (49.0) | |
| Previous | 79 | (53.7) | 100 | (66.2) | 79 | (56.8) | 258 | (59.0) | 0.139 |
| nonvertebral | | | | | | | | | |
| fracture n (% | | | | | | | | | |
| yes) | | | | | | | | | |

| | | | | | |
|---|---|---|---|---|---|
| Baseline vertebral | 0.85 ± 0.14 | 0.89 ± 0.15 | 0.87 ± 0.14 | 0.87 ± 0.14 | 0.053 |
| BMD (mean ± | | | | | |
| SD) | | | | | |
| Dietary calcium | 0.86 ± 0.57 | 0.84 ± 0.54 | 0.80 ± 0.50 | 0.84 ± 0.54 | 0.667 |
| (g/day) | | | | | |
| (mean ± SD) | | | | | |

Abbreviations: N = number of patients randomly assigned to each treatment group; PTH20 = LY333334 20 μg/day; PTH40 = LY333334 40 μg/day; SD = standard deviation; n = number of patients in a category; BMD = bone mineral density.
^{a}1 patient was excluded from the body mass index analysis because of a missing value.
^{b}1 patient was excluded from the height analysis because of a missing value.

Results

Compared to placebo, treatment with LY333334 20-μg/day and 40-μg/day in men with primary osteoporosis for a median follow-up of approximately 11 months resulted in statistically significant dose-related increases in lumbar spine bone mineral density (BMD) after only 3 months of treatment, and at all subsequent visits and endpoint (5% and 8%, respectively). Statistically significant increases in BMD compared with placebo were also found at the total hip, and the femoral neck, as well as the whole body. The distal ⅓ radius, containing primarily cortical bone, and the ultradistal radius showed no statistically significant changes in BMD compared with placebo.

Changes in biochemical markers of bone formation and resorption are consistent with positive, or anabolic, effects of LY333334 on bone. Significant and sustained increases in serum bone-specific alkaline phosphatase (BSAP) and significant increases in procollagen 1 carboxy-terminal propeptide (PICP), representative biochemical markers associated with bone formation, were seen after only 1 month of treatment with LY333334. There was evidence for a pharmacodynamic dose-response in marker concentration, and the maximal increase in PICP was observed within the first 3 months of treatment. Slightly delayed but significant increases in urinary N-telopeptide and urinary free deoxypyridinoline, the biochemical markers of resorption evaluated in this study, were observed for the 20-μg and 40-μg doses of LY333334. This was consistent with increased remodeling, or “recoupling” of bone formation and resorption a few months after the start of treatment.

Nonvertebral Fractures

Although the incidence of nonvertebral fractures was measured, it was not a specified efficacy endpoint. The number of patients reporting at least one incident nonvertebral fracture is tabulated by fracture location in Table 10 (below). The number of fractures was small, and there was no significant treatment difference in the proportion of patients having at least one incident nonvertebral fracture (p=0.670). At individual body sites, the number of fractures was insufficient for a meaningful statistical analysis.

TABLE 10
Nonvertebral Fracture Results-All Randomly Assigned Patients

| | Placebo | PTH20 | PTH40 |
|---|---|---|---|
| | (N = 147) | (N = 150) | (N = 139) |
| Radius | 0 | 1 | 0 |
| Ankle | 0 | 1 | 0 |
| Ribs | 1 | 1 | 0 |
| Other | 3 | 0 | 1 |
| Total Patients^{a} | 3 | 2 | 1 |

Abbreviations: PTH20 = LY333334 20 μg/day, PTH40 = LY333334 40 μg/day; N = nr randomized.
^{a}Patients may have sustained more than one fracture.

Bone Densitometry—Overview

Patients treated with LY333334 20 μg/day and 40 μg/day in study GHAJ had statistically significant increases in lumbar spine BMD of 5.7% and 8.8%, respectively, and significant increases in hip (femoral neck) BMD of 1.4% and 2.9%, respectively, at study endpoint. These increases were statistically significant compared with the approximately 0.5% increase in lumbar spine BMD and 0.4% increase in hip (femoral neck) BMD in the placebo group.

Mean change and mean percent change in BMD from baseline to endpoint (Month 12) for all skeletal sites evaluated for all randomly assigned patients is summarized in Table 11 (below).

Compared with the placebo group, LY333334-treated patients had a statistically significant increase in whole body BMD of approximately 0.5% in both the 20-μg and 40 μg groups that was statistically significant compared with a decrease of 0.3% in the placebo group. Compared with the placebo group, distal ⅓ radius (forearm) and ultradistal radius BMD was unchanged in both the 20-μg and 40-μg groups.

In the placebo group, about 39.9% of the patients had a decrease in lumbar spine BMD at study endpoint. A decrease in vertebral BMD was seen in only 7.1% and 6.2% of patients treated with LY333334 20 μg and 40 μg, respectively. An increase in lumbar spine BMD of 5% or more was observed in 9.8% of patients in the placebo group. In contrast, this increase in lumbar spine BMD was seen in 54.6% of patients in the LY333334 20-μg group and 70.5% of those in the LY333334 40-μg group.

Patients in the hypogonadal and idiopathic subgroups did not differ significantly in their lumbar spine BMD response to LY333334 treatment.

TABLE 11
Summary of Bone Mineral Density
Mean Actual Change and Mean Percent Change from Baseline to Endpoint ± Standard Deviation
All Randomly Assigned Patients

| | | | | P-Value (Treatment Comparison) | | | |
|---|---|---|---|---|---|---|---|
| | Placebo | PTH20 | PTH40 | | Placebo | Placebo | PTH20 |
| Variable | (N = 147) | (N = 151) | (N = 139) | Overall | vs PTH20 | vs PTH40 | vs PTH40 |
| Lumbar Spine (L-1 through L-4) | | | | | | | |
| n | 143 | 141 | 129 | — | — | — | — |
| Mean baseline (g/cm²) | 0.85 ± 0.14 | 0.89 ± 0.15 | 0.87 ± 0.14 | 0.016 | 0.005 | NS | NS |
| Mean change (g/cm²) | 0.01 ± 0.03 | 0.05 ± 0.04 | 0.07 ± 0.05 | <0.001 | <0.001 | <0.001 | <0.001 |
| Mean percent change | 0.54 ± 4.19 | 5.73 ± 4.46 | 8.75 ± 6.25 | <0.001 | <0.001 | <0.001 | 0.001 |
| Total Hip | | | | | | | |
| n | 137 | 135 | 125 | | | | |
| Mean baseline (g/cm²) | 0.83 ± 0.11 | 0.84 ± 0.10 | 0.83 ± 0.11 | NS | NS | NS | NS |
| Mean change (g/cm²) | 0.00 ± 0.02 | 0.01 ± 0.02 | 0.02 ± 0.03 | <0.001 | 0.017 | <0.001 | 0.017 |
| Mean percent change | 0.41 ± 2.77 | 1.14 ± 2.89 | 2.33 ± 4.51 | <0.001 | 0.040 | <0.001 | 0.011 |
| Femoral Neck | | | | | | | |
| n | 137 | 135 | 125 | — | — | — | — |
| Mean baseline (g/cm²) | 0.70 ± 0.11 | 0.71 ± 0.10 | 0.70 ± 0.11 | NS | NS | NS | NS |
| Mean change (g/cm²) | 0.00 ± 0.03 | 0.01 ± 0.03 | 0.02 ± 0.04 | <0.001 | 0.013 | <0.001 | 0.032 |
| Mean percent change | 0.36 ± 3.95 | 1.44 ± 3.61 | 2.85 ± 6.07 | <0.001 | 0.038 | <0.001 | 0.016 |
| Trochanter | | | | | | | |
| n | 137 | 135 | 125 | — | — | — | — |
| Mean baseline (g/cm²) | 0.65 ± 0.11 | 0.66 ± 0.10 | 0.65 ± 0.12 | NS | NS | NS | NS |
| Mean change (g/cm²) | 0.01 ± 0.02 | 0.01 ± 0.03 | 0.01 ± 0.03 | NS | NS | 0.024 | NS |
| Mean percent change | 0.95 ± 3.40 | 1.25 ± 4.15 | 1.98 ± 5.16 | NS | NS | 0.044 | NS |
| Intertrochanter | | | | | | | |
| n | 137 | 135 | 125 | — | — | — | — |
| Mean baseline (g/cm²) | 0.96 ± 0.13 | 0.98 ± 0.13 | 0.97 ± 0.14 | NS | NS | NS | NS |
| Mean change (g/cm²) | 0.00 ± 0.03 | 0.01 ± 0.03 | 0.02 ± 0.04 | <0.001 | 0.030 | <0.001 | 0.041 |
| Mean percent change | 0.48 ± 2.93 | 1.20 ± 3.07 | 2.32 ± 4.57 | <0.001 | NS | <0.001 | 0.024 |
| Ward's Triangle | | | | | | | |
| n | 137 | 135 | 125 | — | — | — | — |
| Mean baseline (g/cm²) | 0.51 ± 0.12 | 0.51 ± 0.11 | 0.50 ± 0.13 | NS | NS | NS | NS |
| Mean change (g/cm²) | 0.00 ± 0.04 | 0.01 ± 0.04 | 0.03 ± 0.05 | <0.001 | 0.044 | <0.001 | 0.003 |
| Mean percent change | 0.71 ± 8.64 | 2.48 ± 7.20 | 6.19 ± 10.21 | <0.001 | NS | <0.001 | 0.001 |
| Whole body^{a} | | | | | | | |
| n | 87 | 84 | 83 | — | — | — | — |
| Mean baseline (g/cm²) | 1.07 ± 0.09 | 1.08 ± 0.09 | 1.07 ± 0.08 | NS | NS | NS | NS |
| Mean change (g/cm²) | −0.00 ± 0.03 | 0.01 ± 0.03 | 0.01 ± 0.03 | 0.025 | 0.026 | 0.015 | NS |
| Mean percent change | −0.33 ± 2.51 | 0.50 ± 2.99 | 0.54 ± 2.45 | 0.039 | 0.039 | 0.021 | NS |
| Ultradistal Radius (Forearm)^{a} | | | | | | | |
| n | 93 | 89 | 85 | — | — | — | — |
| Mean baseline (g/cm²) | 0.43 ± 0.06 | 0.44 ± 0.07 | 0.43 ± 0.06 | NS | NS | NS | NS |
| Mean change (g/cm²) | −0.00 ± 0.01 | −0.00 ± 0.01 | 0.00 ± 0.02 | NS | NS | NS | NS |
| Mean percent change | −0.53 ± 3.28 | −0.40 ± 3.15 | 0.54 ± 5.98 | NS | NS | NS | NS |
| Distal Radius (Forearm)^{a} | | | | | | | |
| N | 93 | 89 | 85 | — | — | — | — |
| Mean baseline (g/cm²) | 0.78 ± 0.12 | 0.78 ± 0.12 | 0.77 ± 0.11 | NS | NS | NS | NS |
| Mean change (g/cm²) | −0.00 ± 0.02 | −0.00 ± 0.02 | −0.01 ± 0.02 | NS | NS | NS | NS |
| Mean percent change | −0.18 ± 2.03 | −0.47 ± 2.21 | −0.67 ± 2.36 | NS | NS | NS | NS |

Abbreviations: N = number of patients randomly assigned to each treatment group; PTH20 = LY333334 20 μg/day; PTH40 = LY333334 40 μg/day; vs = versus; n = maximum number of patients with a baseline and at least one postbaseline measurement; NS = not significant.
^{a}Whole body and radius bone mineral density were measured in a subset of patients.

Skeletal Site-Specific Results

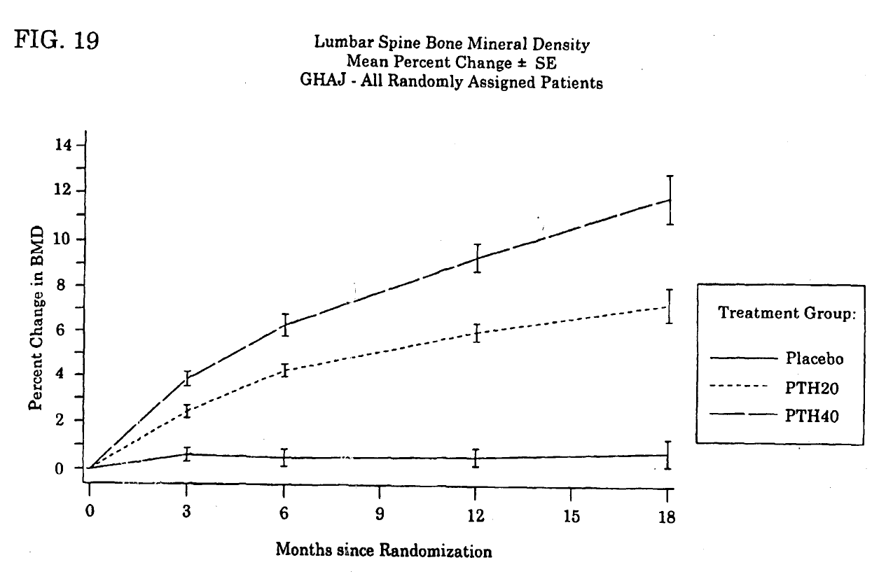

Total (L-1 through L-4) lumbar spine BMD mean percent changes from baseline by visit are graphically depicted in FIG. 19. A statistically significant difference was observed in lumber spine BMD among the treatment groups (p=0.016) at baseline. Unadjusted p-values from multiple comparison tests of the baseline measurements indicate that the placebo group had a lower BMD than the 20-μg group (p=0.005). An ANCOVA was performed on the endpoint BMD using baseline BMD as covariate. The ANCOVA showed significant difference for change-from-baseline BMD among the treatment groups after adjusting for baseline measurements (p<0.001).

BMD increased significantly (p<0.001) in both the 20-μg and 40-μg groups compared with placebo at Month 12, and at each visit where it was assessed (p<0.001 for all comparisons). The difference in BMD between the 20-μg group and placebo was 5.49% at Month 12. The difference between the 40-g group and placebo was 8.83% at Month 12. The LY333334 groups were statistically significantly different from each other at all times (p<0.001 for all visits).

As shown in FIG. 19, statistically significant increases in BMD occurred rapidly. In the placebo group, lumbar spine BMD increased significantly by 0.61% above baseline at Month 3 (p=0.030) but was not changed significantly at Month 12. The lumbar spine BMD increased significantly in the 20-μg group by 2.44% at Month 3 (p<0.001), 4.29/at Month 6 (p<0.001), and 6.07% at Month 12 (p<0.001). The lumbar spine BMD increased significantly in the 40-μL group by 3.87% at Month 3 (p<0.001), 6.33% at Month 6 (<0.001), and 9.41% at Month 12 (p<0.001).

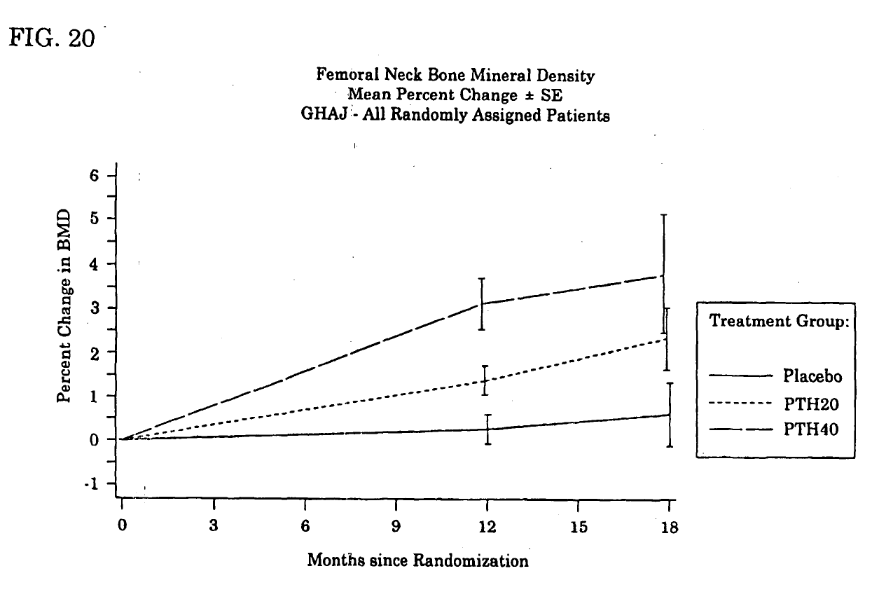

Femoral neck BMD mean percent changes from baseline by visit are graphically depicted in FIG. 20. There was no statistically significant difference among treatment groups for femoral neck BMD at baseline using ANOVA. The treatment group difference was statistically significant at Month 12 (p<0.001). In addition, each LY333334 group had significantly greater increases in femoral neck BMD than the placebo grout at Month 12 (p=0.339 for the 20-μg group and p<0.001 for the 40-μg group). The LY333334 groups were significantly different from each other at Month 12 (p=0.004).

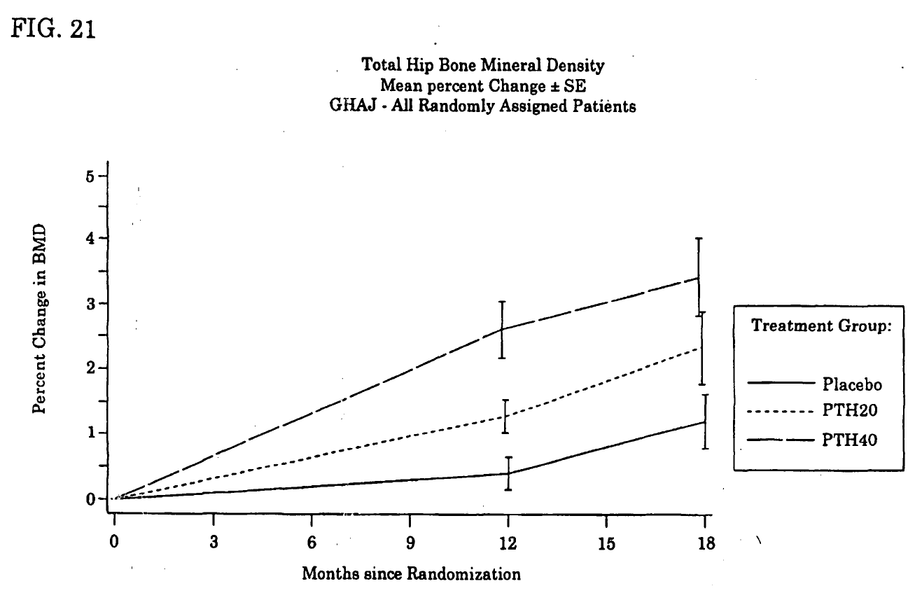

Total hip BMD mean percent changes from baseline by visit are graphically depicted in FIG. 21. There was no statistically significant difference among treatment groups for total hip BMD at baseline using ANOVA. The treatment group difference was statistically significant at Month 12 (p<0.001). In addition, each LY333334 group had significantly greater increases in total hip BMD than the placebo group at Month 12 (p=0.023 for the 20-μg group and p<0.001 for the 40-μg group). The LY333334 groups were significantly different from each other at Month 12 (p=0.006).

Biochemical Markers of Bone Formation and Resorption Serum Procollagen I Carboxy-Terminal Propeptide (Serum PICP).

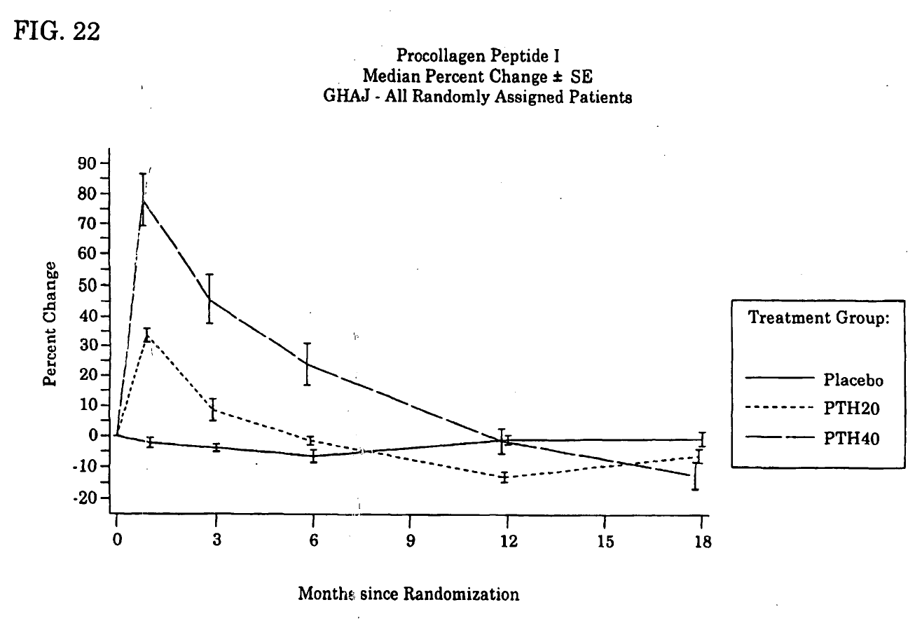

Percent changes in serum PICP are depicted graphically by visit and dose in FIG. 22. There was no statistically significant difference among treatment groups for serum PICP levels at baseline. There were overall statistically significant differences among the three treatment groups in PICP at Months 1, 3, 6, and 12 (p<0.001). The percent increase from baseline in serum PICP for the 20-μg group was statistically significantly larger than for the placebo group at Months 1 and 3 (p<0.001). At Month 12, PICP for the 20-μg group was decreased compared with baseline. This change was statistically significant compared with the placebo group (p<0.001). The percent increase from baseline for the 40-μg group was statistically significantly larger than for the placebo group at Months 1, 3, and 6 (p<0.001). At Month 12, serum PICP for the 40-μg group was slightly decreased compared with baseline. This change was not statistically significant compared with the placebo group. The change for the 40-μg group was statistically significantly greater than the 20-μg group at Months 1, 3, 6, and 12 (p≦0.001).

The LY333334 treatment groups showed a rapid increase in serum PICP to peak concentrations (33.7% above baseline for the 20-μg group and 78.0% above baseline for the 40-μg group) at Month 1 (p<0.001 for both comparisons). Overall, the timing and pattern of changes in this marker of bone formation in men treated with LY333334 were very similar to those observed in postmenopausal women.

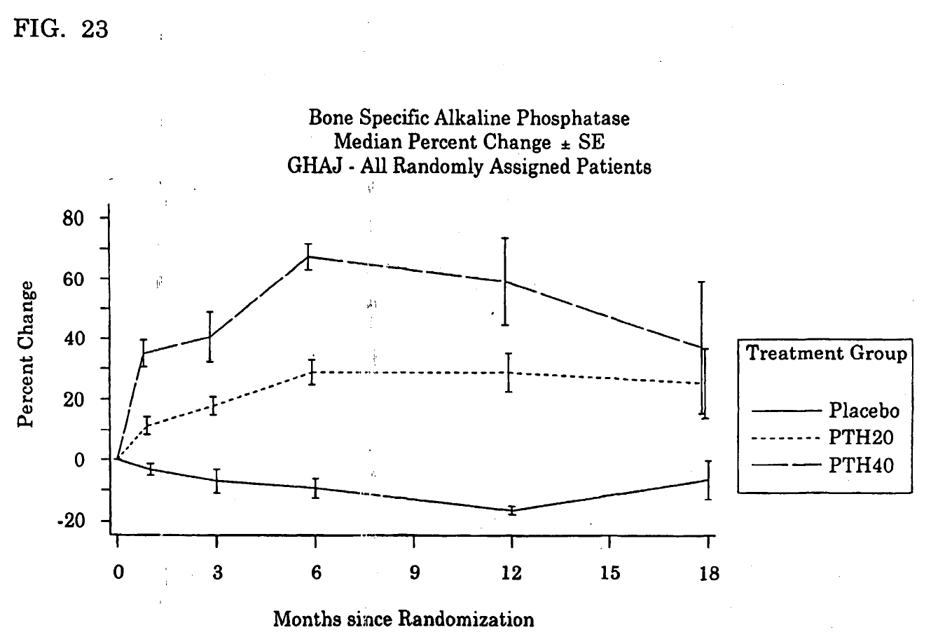

Serum Bone-Specific Alkaline Phosphatase (Serum BSAP). Percent changes in serum BSAP are depicted graphically by visit and dose in FIG. 23. There was no statistically significant difference among treatment groups for serum BSAP levels at baseline. There were overall statistically significant differences among the three treatment groups in percent change of serum BSAP at Months 1, 3, 6, and 12 (p<0.001 for all visits). Both doses of LY333334 produced statistically significantly larger increases in serum BSAP than placebo at Months 1, 3, 6, and 12 (p<0.001 for all visits). Moreover, the increase in the 40-μg group was statistically significantly larger than in the 20-μg group throughout the study (p<0.001 for all visits).

The LY333334 treatment groups showed a statistically significant increase in serum BSAP percent change from baseline at every scheduled visit (p<0.001 for all visits). The increase reached a plateau between Months 6 and 12. At Month 12, the serum BSAP concentration was increased by 28.8% for the 20-μg group (p<0.001) and 59.3% for the 40-μg group (p<0.001).

Overall, the timing and pattern of changes in this marker of bone formation in men treated with LY333334 were very similar to those observed in postmenopausal women.

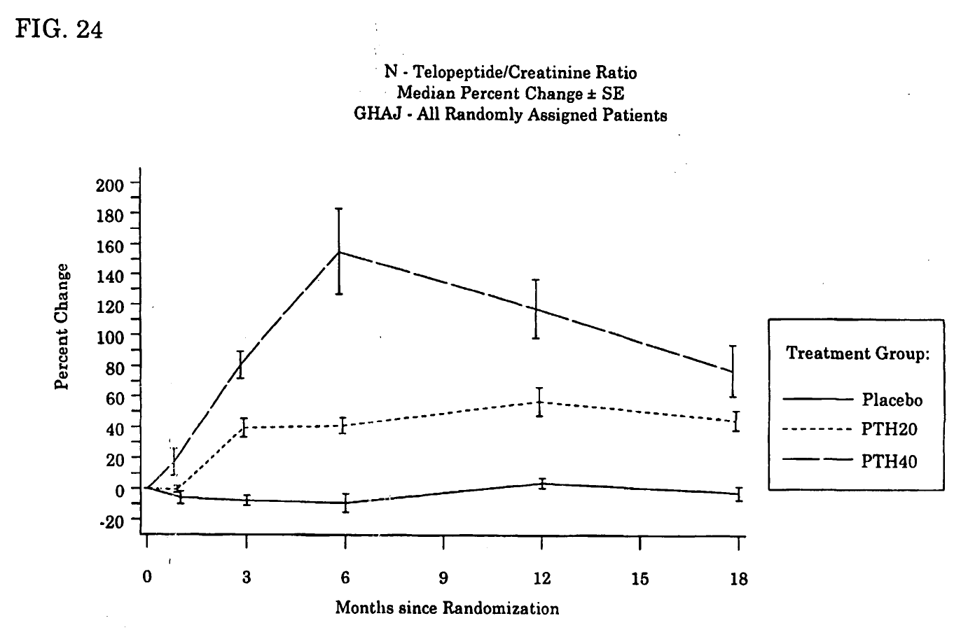

Urinary N-Telopeptide (NTX). Urinary NTX was reported as the ratio of N-telopeptide to creatinine. Percent changes in urinary NTX are depicted graphically by visit in FIG. 24. There was no statistically significant difference among treatment groups for urinary NTX levels at baseline. The overall treatment group differences for urinary NTX were statistically significant at all visits (p≦0.001). The difference between the 20-μg group and placebo was statistically significant at Months 1 through 12 (p=0.040 for Month 1 and p<0.001 for all other visits). The difference between the 40-μg and placebo groups was significant at Months 1 through 12 (p<0.001). The difference between the two LY333334 treatment groups was significant at all visits (p<0.001).

The 20-μg group showed a significant increase in urinary NTX percent change from baseline as early as Month 3 (p<0.001), peaking at approximately 57% at Month 12 (p<0.001). The 40-μg group also showed a significant increase in urinary NTX percent change from baseline at every visit and as early as Month 1 (p<0.001), peaking at approximately 155% at Month 6 (p<0.001). Urinary NTX levels subsequently declined thereafter to approximately 118% over baseline at Month 12 (p<0.001).

Overall, the timing and pattern of changes in this marker of bone resorption in men treated with LY333334 were very similar to those observed in postmenopausal women.

Height

There were no statistically significant differences among treatment groups in mean height at baseline (approximately 173 cm) or at study endpoint. Patients in the placebo, 20-μg, and 40-μg groups showed a mean height decrease of 1.90, 2.20, and 3.25 mm, respectively, at endpoint (all p≦0.001 compared with baseline). Similarly, the by-visit analysis also did not show any statistically significant treatment differences at any visit.

### SUMMARY AND CONCLUSIONS

The efficacy of LY333334 20-μg and 40-μg once daily was demonstrated in is this double-blind, placebo-controlled clinical study in 437 men with osteoporosis. LY333334 and placebo were administered in conjunction with 1000 mg of calcium per day and 400 IU of vitamin D per day supplementation.

Change in BMD was evaluated in patients treated daily for up to 14 months. Vertebral fractures were not assessed, but investigators distinguished nonvertebral fragility fractures from nonvertebral traumatic fractures that would have occurred in an otherwise healthy person. Bone densitometry and measurements of height and bone marker concentration were obtained at scheduled intervals between baseline and endpoint. No statistically significant effects on nonvertebral fracture or height loss were observed in this relatively brief study.

The efficacy of treatment with LY333334 20 μg and 40 μg once daily for up to 15 months was shown by increases in lumbar spine BMD of 5.73% and 8.75%, respectively, increases in hip BMD of 1.14% and 2.33%, respectively, and increases in femoral neck BMD of 1.44% and 2.85%, respectively, at study endpoint. These changes were statistically significant relative to placebo and baseline. Patients in the hypogonadal and idiopathic subgroups did not differ significantly in their lumbar spine BMD response to LY333334 treatment.

As observed in postmenopausal women with osteoporosis, treatment with LY333334 did not significantly increase radius BMD. Compared with the placebo group, distal ⅓ radius(forearm) and ultradistal radius BMD was unchanged in both the 20 μg and 40 μg groups. Nevertheless, treatment of postmenopausal women with osteoporosis with LY333334 under the same conditions has been shown to concurrently reduce the risk of both vertebral and non-vertebral bone fracture. Given the similarities in responses to LY33334 of men and women, in terms of both spinal and non-spinal BMD increases, as well as in bone marker responses described herein, concurrent reductions in the risk of both vertebral and non-vertebral bone fracture similar to those observed in women with osteoporosis are also expected in men with osteoporosis when the women and men are similarly treated with parathyroid hormone.

For LY33334 (i.e., hPTH(1-34)) in particular, in studies by the present applicant the lowest tested dose found to be effective for stimulation of bone formation in human subjects, as indicated by bone markers as disclosed herein, was about 15 μg; 6 μg was found to produce no significant effects. Therefore, treatment of osteoporosis in men or women with hPTH(1-34) preferably should use a daily dose greater than about 6 μg, more preferably at least about 15 μg. Daily doses of hPTH(1-34) of both 20 μg and 40 μg were found to be similarly effective against osteoporosis in both men and women. Higher daily doses of hPTH(1-34) have been used in human subjects previously, although parathyroid hormone has never been shown to reduce the risk of fracture reduction in nonvertebral bone in human subjects, and hPTH(1-34) has not even been shown to reduce vertebral fractures when used without an antiresorptive agent other than calcium or vitamin D (e.g., without gonadal hormone replacement therapy). Therefore, any daily dose of hPTH(1-34) in the range of greater than about 6 μg to at least about 40 μg would be effective for reduction of the risk of both vertebral and nonvertebral fractures, according to the present method of using this form of parathyroid hormone. However, this applicant has found that a daily dose of about 20 μg produced-fewer undesirable side effects in human subjects than a daily dose of about 40 μg. Hence, daily doses above about 40 μg are less preferred than doses of 40 μg of less; and a daily dose of about 20 μg is more preferred than any higher dose from this perspective.

Accordingly, the present findings provide a rational basis for a method for concurrently reducing the risk of both vertebral and non-vertebral bone fracture in a male human subject at risk of or having hypogonadal and idiopathic osteoporosis comprising administering to the subject a parathyroid hormone. Preferably, the parathyroid hormone consists of amino acid sequence 1-34 of human parathyroid hormone; and this hormone is administered without concurrent administration of an antiresorptive agent other than vitamin D or calcium, in a daily dose in the range of about 15 μg to about 40 μg, for at least about 12 months up to about 3 years.

The DXA measured bone mineral area increased significantly in the lumbar spine in both the 20-μg and 40-μg groups when compared with placebo (p<0.001). This increased the denominator for calculated lumbar spine BMD. Comparison of total lumbar spine BMD and BMC results suggest that DXA measurements of change in BMD are conservative estimates of the skeletal effects of treatment with LY333334. Compared with the placebo group, patients treated with LY333334 20 μg/day and 40 μg/day had significant increases in lumbar spine BMC of 7% and 10%, respectively, and increases in hip (femoral neck) BMC of 1% and 3% respectively, at study endpoint. Increases in hip (femoral neck) BMC and in total body BMC at study endpoint were significantly greater than placebo in the 40 kg group but not in the 20 μg group. Compared with the placebo group, ultradistal radius BMD was unchanged in both the 200 μg and 40 μg groups, and the distal ⅓ radius (forearm) BMD was unchanged in the 20 μg group, but was significantly decreased by 1.0% in the 40 μg group. Compared with the placebo group, the LY333334-treated patients had an increase in whole body BMC of approximately 0.9% in the 20 μg group and a statistically significant increase of 1.3% in the 40 μg group.

The changes in biochemical markers of bone formation and resorption were consistent with the known anabolic effects of PTH treatment on bone remodeling. Significant and sustained increases in serum BSAP and serum PICP, markers associated with osteoblast activity and active bone formation, were observed after the first month of treatment with LY333334. The levels of all bone markers tended to regress towards baseline after discontinuation of LY333334, although only serum PICP levels had returned to baseline by the closeout visit. Despite the variable interval between discontinuation of treatment and this visit, the data suggest that the anabolic effect of LY333334 treatment on bone metabolism does not continue after treatment is withdrawn.

### EXAMPLE 6

### Prediction of Bone Mineral Density Response to LY333334 Treatment in Women and Men by Monitoring Biochemical Markers

Data from studies in Examples 1 and 5 above were further analyzed to develop more detailed models for the use of bone markers in monitoring and predicting effects of PTH on clinically significant correlates of efficacy in the treatment of osteoporosis, such as bone mineral density (BMD). Population pharmacodynamic (PD) models were developed for total lumbar spine BMD, and the following biochemical markers of bone formation and resportion: PICP, BSAP, NTX, and DPD. The final treatment-response model for total lumbar spine was used to calculate BMD values at 12 months of treatment for each patient, based on the individual's parameter estimates (empirical Bayesian estimate). These predicted BMD measurements were merged with the observed BCM values, at baseline, 1 month, and 3 months of treatment, for patients who completed at least 12 months of LY333334 treatment. A neural network was developed to characterize the relationship between BCM values at 1 and 3 months and response to treatment, as measured by change in total lumbar spine BMD.

Methods

Table 12 (below) lists covariates examined in pharmacodynamic analyses.

TABLE 12
Patient Factors Assessed in the Population
Pharmacodynamic Analyses

| | |
|---|---|
| LY333334 treatment | 25-hydroxyvitamin D at screening |
| group | |
| Gender | 1,25-dihydroxyvitamin D ^{a} |
| Injection site | Bone-Specific Alkaline Phosphatase ^{a} |
| (abdomen or thigh) | |
| Age | Urinary Free Deoxypyridinoline/Creatinine ratio^{a} |
| Years postmenopausal | Urinary N-telopeptide/Creatinine ratio ^{a} |
| Ethnic origin | Thyroid-stimulating Hormone at screening |
| Body weight | Endogenous PTH (1-84) at screening |
| Body Mass Index | Procollagen I Carboxy-Terminal Propeptide^{a} |
| Alcohol use | Total lumbar spine bone mineral density ^{a} |
| Smoking status | Free Testosterone ^{a} |

^{a} Only baseline value used in pharmacodynamic covariate analyses.

Datasets for Pharmacodynamic Analyses

Bone mineral density and biochemical marker measurements were combined with demographic data and clinical laboratory test results using SAS® to produce the datasets used in the population pharmacodynamic analyses.

Datasets were prepared for the population analysis of total lumbar spine BMD, and biochemical markers of bone formation and resorption (BCM). The BCMs for bone formation were serum concentrations of procollagen I carboxy-terminal propeptide (PICP) and bone-specific alkaline phosphatase (BSAP); the BCMs for bone resorption were urinary excretion of N-telopeptide (NTX) and free deoxypyridinoline (DPD), normalized for creatinine excretion. Patients with missing baseline values for a pharmacodynamic endpoint were omitted from the respective dataset. Table 13 (below) provides a summary of patients and observations included in the pharmacodynamic datasets.

TABLE 13
Data Included in the Pharmacodynamic Analyses

| | | Number | Number of | |
|---|---|---|---|---|
| Pharmacodynamic | LY333334 | of | Obser- | Patients |
| Endpoint | Treatment Groups | Patients | vations | Excluded^{a} |
| Total Lumbar Spine | Placebo, 20-μg, | 1927 | 6724 | 34 |
| BMD | and 40-μg | | | |
| Procollagen I | 20-μg and 40-μg | 623 | 2683 | 15 |
| Carboxy-terminal | | | | |
| Propeptide | | | | |
| Bone-specific | 20-μg and 40-μg | 621 | 2673 | 17 |
| Alkaline | | | | |
| Phosphatase | | | | |
| Urinary N- | 20-μg and 40-μg | 616 | 2625 | 18 |
| telopeptide | | | | |
| Urinary free | 20-μg and 40-μg | 613 | 2608 | 20 |
| Deoxypyridinoline | | | | |

^{a}Due to missing baseline value for pharmacodynamic endpoint

Data Analysis Methods

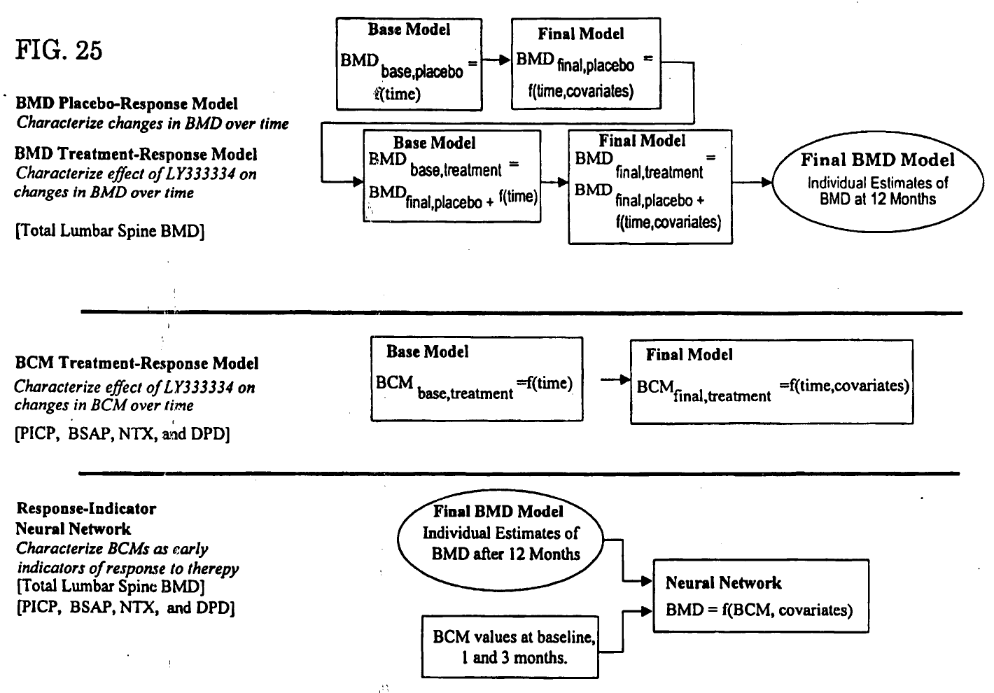

An outline of the pharmacodynamic analyses performed is provided in FIG. 25. The spine BMD placebo-response model characterized change in total lumbar spine BMD over time in osteoporotic patients taking calcium and vitamin D supplements. The BMD treatment-response model was used to characterize change in total lumbar spine BMD during the course of treatment and to identify patient factors influencing response to therapy. This model was also used to provide individual estimates of change in BMD at 12 months. The BCM treatment-response models characterized changes in PICP, BSAP, NTX, and DPD, during the course of treatment.

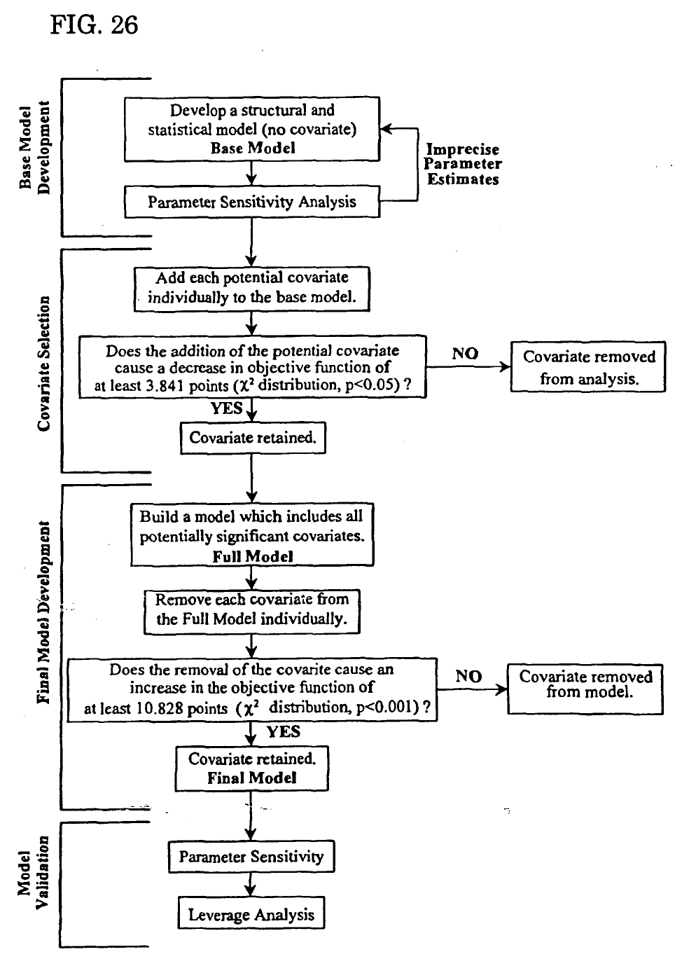

The general process used for pharmacodynamic model development in each of these analyses is shown in FIG. 26. The individual estimates of change in spine BMD from the final treatment-response model were combined with observed BCM values to develop the response-indicator neural network. The neural network was used to evaluate change in the biochemical markers as early indicators of change in total lumbar spine BMD.

BCM Response-Indicator Neural Network

Change in total lumbar spine BMD at 12 months of treatment was calculated from the post-hoc BMD estimates for each patient from the final spine BMD treatment-response model. These BMD estimates were combined with observed BCM values at baseline, 1, and 3 months for all patients completing at least 12 months of LY333334 therapy.

Neural networks were used to evaluate the biochemical markers as potential indicators of bone mineral density response to LY333334 treatment. The relationship between change in biochemical marker values and change in spine BMD is complex and the appropriate model structure is unknown. The neural network approach was chosen to avoid the a priori assumption of a model form. A proprietary artificial neural network program developed at Eli Lilly and Company (described in Wikel J, Dow E, Heathman M. 1996. Interpretive Neural Networks for QSAR. Network Science [available on-line]) was used to evaluate the BCM values as predictors of change in spine BMD. Other back-propagation networks which are known in the art and commercially available also would provide similar results. The BCM values, as well as significant patient factors from the final spine BMD treatment-response model, were used as inputs to the neural network. The network was trained to predict change in total lumbar spine BMD.

Results

The increase in PICP concentration at 1 month after initiation of treatment was the most significant predictor of increase in total lumbar spine BMD at 12 months. Higher PICP concentrations at baseline were also associated with a greater increase in spine BMD. High BSAP concentrations at 3 months and increased age were both predictive of greater increase in spine BMD for postmenopausal women. LY333334 treatment group also influenced response to therapy, with patients in the 40-μg having a greater increase in spine BMD.

Many patients with modest increases in PICP at 1 month showed substantial increases in BMD. However, all patients with baseline PICP concentrations greater than 100 pM and an increase in PICP concentration greater than 100 pM, showed at least a 4.3% increase in total lumbar spine BMD. The mean increase in these patients was 13.6%, compared to 8.2% for patients who did not meet these criteria.

Total Lumbar Spine BMD

Patient Characteristics. The neural network evaluation of biochemical markers and total lumbar spine BMD included data from 276 postmenopausal women whose age ranged from 49 to 84 years at study entry and who weighed between 43.1 and 120 kg. Baseline measurements for spine BMD ranged from 0.38 to 1.31 g/cm². The analysis also included data from 210 osteoporotic men whose age ranged from 32 to 84 years at study entry and who weighed between 47.2 and 120.9 kg. Baseline measurements for spine BMD ranged from 0.59 to 1.34 g/cm². The range and mean values of age, weight, baseline spine BMD and for the biochemical markers at baseline are shown in Table 14 (below).

TABLE 14
Demographics at Study Entry, Baseline Spine Bone Mineral Density Values, and Baseline Biochemical Markers Values

| | | | | Body | Spine | | | | | | NTX | DPD |
|---|---|---|---|---|---|---|---|---|---|---|---|---|
| | LY333334 | Age | | Weight | BMD | | PICP | | BSAP | | (nmBCE/ | (nmol/ |
| Study | Treatment Group | (yr) | | (kg) | (g/cm²) | | (pM) | | (pM) | | mmol) | mmol) |
| GHAC | 20-μg/day | | | | | | | | | | | |
| | Range | 49-81 | | 43.1-90.5 | 0.45-1.25 | | 52-255 | | 2.0-43.6 | | 7.7-143.2 | 2.2-16.1 |
| | Mean (% CV) | 68 | (8.8%) | 65.2 (15.5%) | 0.81 | (20.7%) | 116.7 | (30.5%) | 12.5 | (60.1%) | 48.2 (51.4%) | 7.1 (36.6%) |
| | 5^{th}-95^{th} Percentiles | 59-78 | | 49.7-82.0 | 0.55-1.09 | | 74-180 | | 3.4-26.8 | | 18.2-88.4 | 3.3-12.2 |
| | n^{a} | 143 | | 143 | 143 | | 143 | | 143 | | 143 | 143 |
| | 40-μg/day | | | | | | | | | | | |
| | Range | 50-84 | | 45.0-120.0 | 0.38-1.31 | | 60-415 | | 2.4-37.7 | | 6.8-214.3 | 1.1-22.7 |
| | Mean (% CV) | 69 | (10.1%) | 66.9 (17.7%) | 0.85 | (20.3%) | 118.2 | (34.0%) | 12.2 | (58.1%) | 46.9 (61.7%) | 6.9 (41.0%) |
| | 5^{th}-95^{th} Percentiles | 57-79 | | 50.0-88.5 | 0.60-1.12 | | 73-181 | | 4.5-26.0 | | 16.7-92.2 | 3.8-10.8 |
| | n^{a} | 133 | | 133 | 133 | | 133 | | 133 | | 133 | 133 |
| GHAJ | 20-μg/day | | | | | | | | | | | |
| | Range | 32-84 | | 47.2-102.5 | 0.60-1.29 | | 55-294 | | 2.9-34.9 | | 8.8-131.5 | 0.5-11.3 |
| | Mean (% CV) | 59 | (22.2%) | 76.2 (14.8%) | 0.90 | (17.2%) | 128.7 | (33.4%) | 11.0 | (45.6%) | 39.2 (55.8%) | 4.8 (39.4%) |
| | 5^{th}-95^{th} Percentiles | 37-80 | | 60.0-94.2 | 0.68-1.20 | | 80-197 | | 3.8-19.1 | | 14.7-80.3 | 2.7-8.1 |
| | n^{a} | 112 | | 112 | 112 | | 112 | | 112 | | 112 | 112 |
| | 40-μg/day | | | | | | | | | | | |
| | Range | 32-82 | | 47.6-120.9 | 0.59-1.34 | | 55-235 | | 2.0-25.9 | | 9.2-136.6 | 0.3-12.6 |
| | Mean (% CV) | 57 | (21.5%) | 74.9 (16.7%) | 0.86 | (15.9%) | 125.5 | (31.1%) | 11.5 | (44.9%) | 36.7 (60.8%) | 4.5 (41.2%) |
| | 5^{th}-95^{th} Percentiles | 36-75 | | 58.7-94.9 | 0.66-1.07 | | 78-190 | | 4.2-20.3 | | 14.7-82.0 | 1.7-7.6 |
| | n^{a} | 98 | | 98 | 98 | | 98 | | 98 | | 98 | 98 |

Abbreviation: BMD = bone mineral density; PICP = procollagen I carboxy-terminal propeptide; BSAP = bone-specific alkaline phosphatase; NTX = urinary N-telopeptide; DPD = urinary free deoxypyridinoline; CV = coefficient of variation.
^{a}n = Number of patients included in the neural network analysis.

Neural Network Analysis. A total of 486 individual estimates of spine BMD at 12 months were available for analysis from patients for whom biochemical marker values were available. The biochemical marker evaluations at baseline, 1 month, and 3 months were combined with the significant patient factors identified in the final treatment response model; LY333334 treatment group, gender, baseline spine BMD, age at study entry, endogenous PTH(1-84) at screening. Thus, 17 patient factors and BCM values were included in the neural network analysis. A full network was first constructed containing all 17 patient factors. The network was then re-evaluated with each patient factor removed individually from the full network. The least significant patient factor was then removed and the process repeated. The final neural network contains only those patient factors whose removal significantly degrades the network fit.

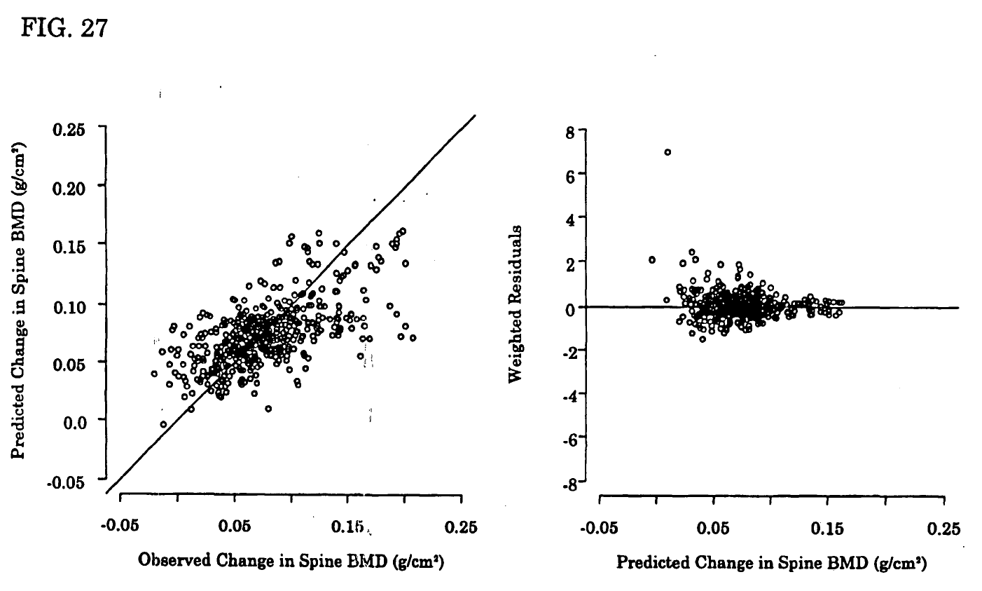

Final Neural Network. The final neural network contained LY333334 treatment group, gender, age at study entry, PICP concentration at 1 month, PICP concentration at baseline, and BSAP concentration at 3 months. Goodness-of-fit of the final network is represented by agreement between predicted and observed BMD values, as well as by weighted residuals (FIG. 27).

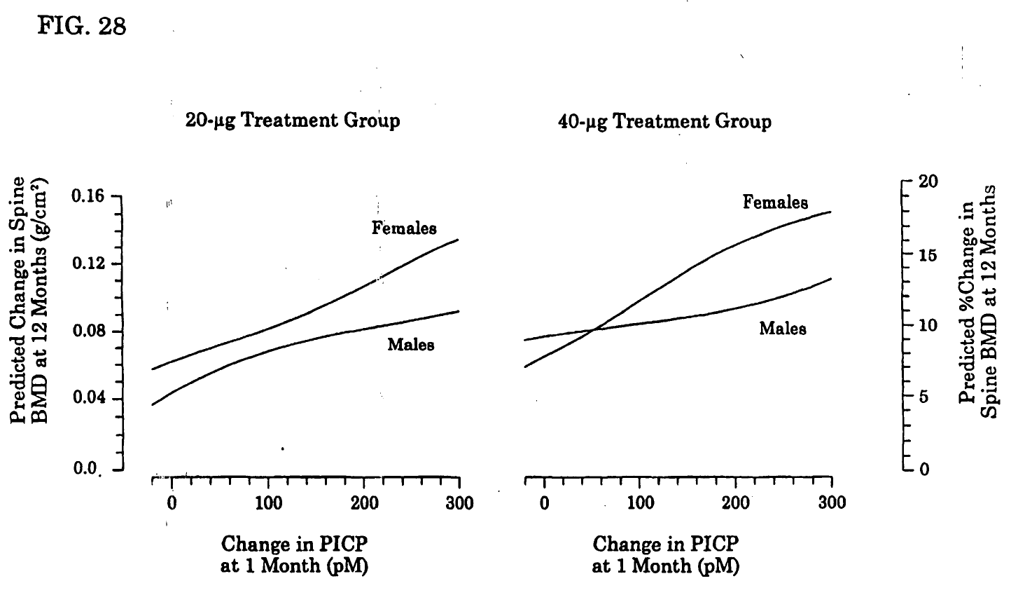

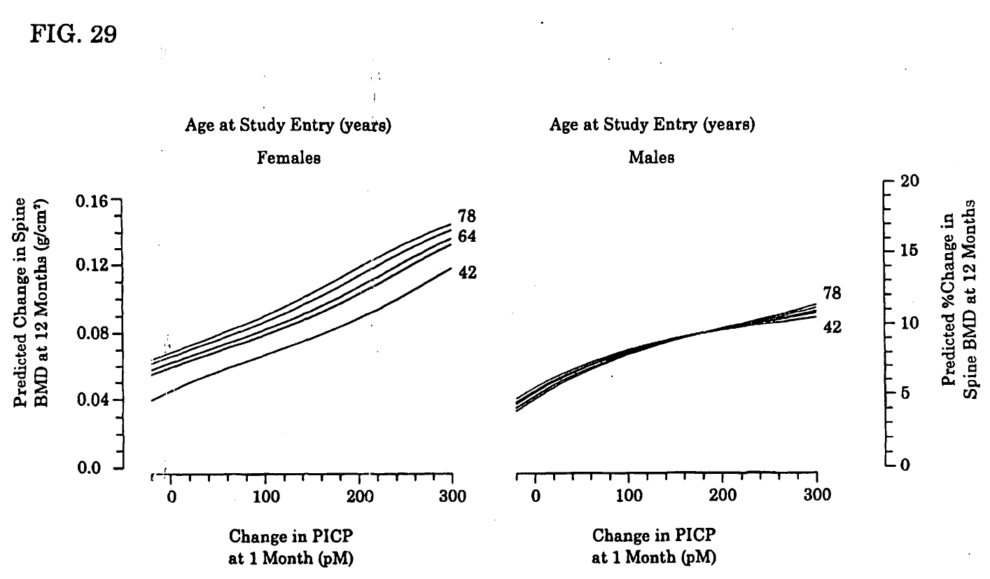

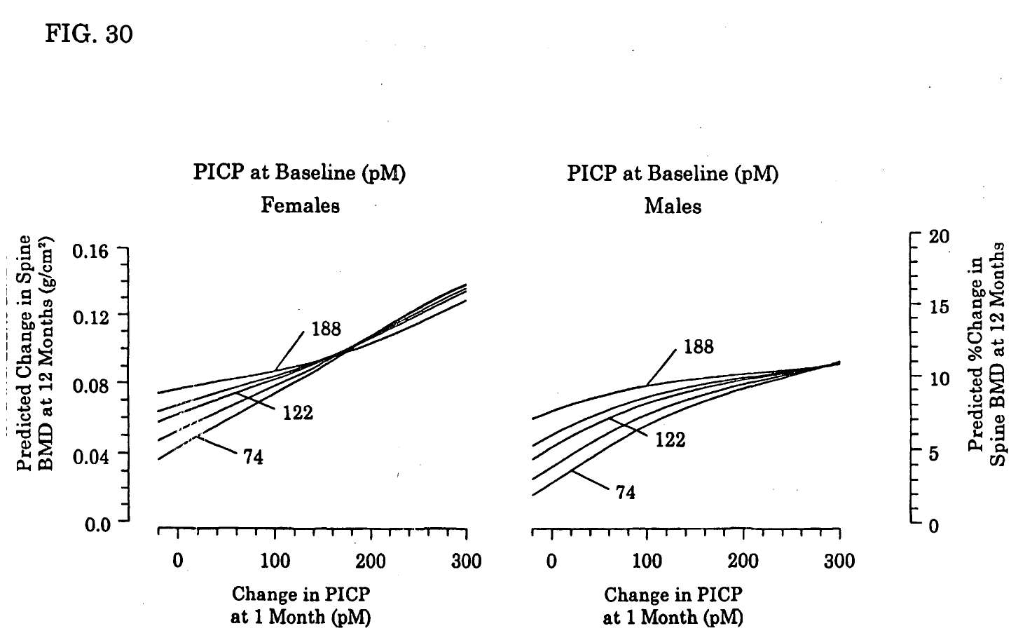

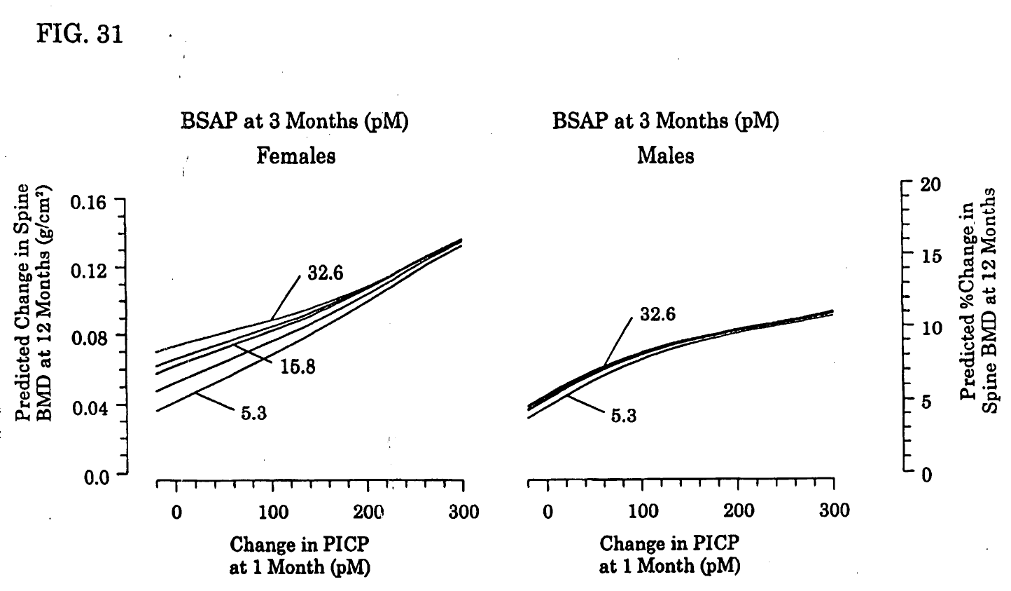

The predicted effect of each patient factor on the change in spine BMD is described in Table 15 and illustrated in FIGS. 28-31. In summary, the network predicts a greater increase in spine BMD for patients with a larger increase in PICP at 1 month of treatment. This relationship is more pronounced in female patients. Patients with higher baseline PICP concentrations are also predicted to have a greater increase in spine BMD. Postmenopausal women with high BSAP concentrations at 3 months and older postmenopausal women were predicted to have greater response to LY333334 treatment.

TABLE 15
Patient factors in Final Neural Network,
Total Lumbar Spine Bone Mineral Density

| Patient Factor | Effect on Change in BMD | |
|---|---|---|
| Change in PICP at | Greater Increase | Greater increase in BMD |
| 1 Month | | |
| PICP Concentra- | Greater concentration | [symbol] Greater increase in BMD |
| tion at Baseline | | |
| BSAP Concentra- | Higher Concentration | [symbol] Greater increase in BMD |
| tion at 3 Months | in postmenopausal | |
| | women | |
| Age at Study | Older postmenopausal | [symbol] Greater increase in BMD |
| Entry | women | |
| LY333334 Treat- | 40-μg Dose | [symbol] Greater increase in BMD |
| ment Group | | |

Abbreviations: BMD = bone mineral density; PICP = procollagen I carboxy-terminal propeptide; BSAP = bone-specific alkaline phosphatase.

Significance of Patient Factors in Final Network. The relative significance of the patient factors in the final neural network was assessed by removing each individually to construct a set of reduced networks. The mean-squared-error (MSE) of the network predictions was calculated for each reduced network and compared to the final network. The results are summarized in Table 16.

TABLE 16
Significance of Patient factors in Final Neural
Network, Total Lumbar Spine Bone Mineral Density

| | Change in MSE of Network |
|---|---|
| Patient Factor | Predictions |
| LY333334 Treatment Group | 0.0000945 |
| Gender | 0.0001183 |
| Age at Study Entry | 0.0001082 |
| PICP at Baseline | 0.0001280 |
| PICP at 1 Month | 0.0001801 |
| BSAP at 3 Months | 0.0000512 |

Abbreviations: PICP = procollagen I carboxy-terminal propeptide; BSAP = bone-specific alkaline phosphatase; MSE = mean-squared-error.

The most significant patient factors in the final network were PICP at 1 month, and PICP at baseline. This suggests that the change from baseline in PICP at 1 month is the most significant factor in predicting change in total lumbar spine BMD.

Relationship between Biochemical Markers of Bone Formation and Change in Total Lumbar Spine BMD. The increase in PICP concentration at 1 month after initiation of treatment was the most significant predictor of increase in total lumbar spine BMD at 12 months. Higher PICP concentrations at baseline were also associated with a greater increase in spine BMD. High BSAP concentrations at 3 months were also predictive of greater increase in spine BMD for postmenopausal women.

Procollagen I Carboxy-terminal Propeptide. Many patients with modest increases in PICP at 1 month showed substantial increases in BMD. However, all postmenopausal women with baseline PICP concentrations greater than 100 pM and an increase in PICP concentration greater than 100 pM, showed at least a 5.9% increase in total lumbar spine BMD. The mean increase in these women was 16.0%, compared to 8.8% for women who did not meet these criteria. All male patients with baseline PICP concentrations greater than 100 pM and an increase in PICP concentration greater than 100 pM, showed at least a 4.3% increase in total lumbar spine BMD. The mean increase in these patients was 10.8%, compared to 7.4% for men who did not meet these criteria.

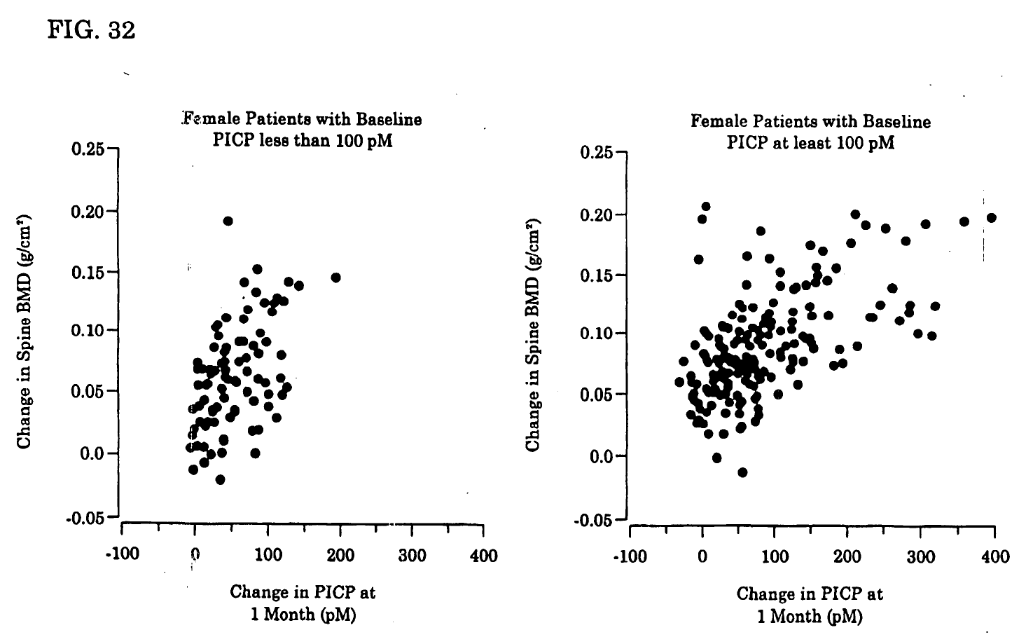

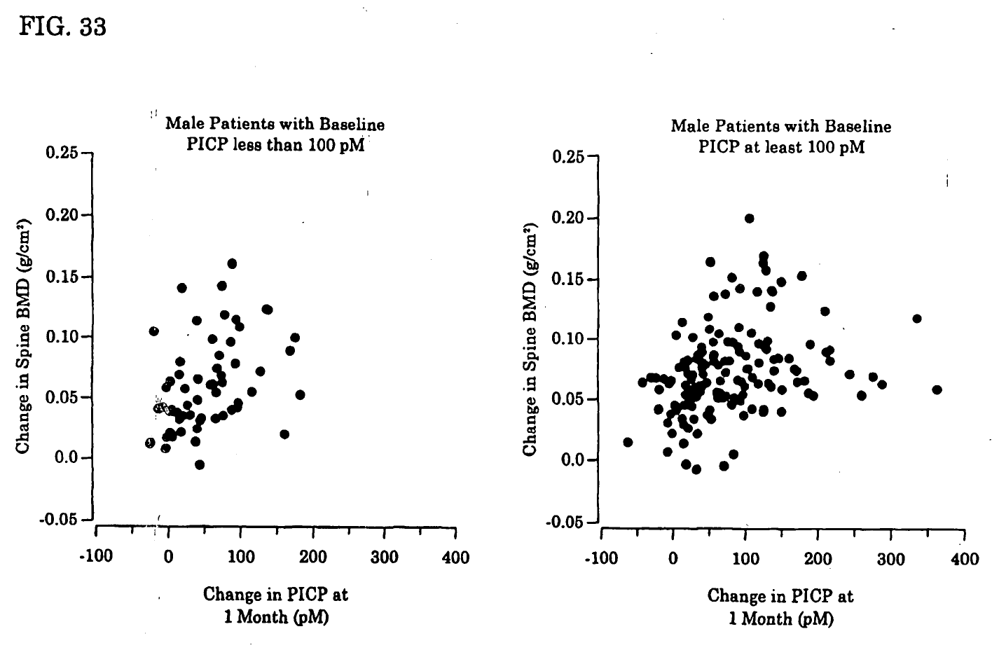

The relationship between change in PICP at 1 month and change in spine BMD at 12 months is shown in FIGS. 32 and 33 for both female and male patients (respectively) with baseline PICP values above and below 100 pM. The effect of PICP on change in spine BMD is further illustrated in Table 17.

TABLE 17
Effect of PICP on Change In Spine Bone Mineral Density

| | | % Change in Spine BMD at 12 Months | | | |
|---|---|---|---|---|---|
| | Change In PICP at 1 | Baseline PICP | | Baseline PICP | |
| Gender | Month | <100 pM | | ≧100 pM | |
| Females | <50 pM | | | | |
| | 5^{th}-95^{th} | −0.7-12.4 | | 2.9-17.3 | |
| | Percentiles | | | | |
| | Mean (% CV) | 5.7 | (81.4%) | 9.0 | (62.0%) |
| | N | 47 | | 70 | |
| | 50-99 pM | | | | |
| | 5^{th}-95^{th} | 2.2-19.0 | | 3.6-18.8 | |
| | Percentiles | | | | |
| | Mean (% CV) | 9.5 | (54.2%) | 10.4 | (45.2%) |
| | N | 26 | | 58 | |
| | 100-149 pM | | | | |
| | 5^{th}-95^{th} | 2.9-16.1 | | 6.9-19.7 | |
| | Percentiles | | | | |
| | Mean (% CV) | 10.4 | (46.0%) | 12.6 | (32.2%) |
| | N | 13 | | 23 | |
| | ≧150 pM | | | | |
| | 5^{th}-95^{th} | — | | 11.9-31.1 | |
| | Percentiles | | | | |
| | Mean (% CV) | 13.7 | (—) | 18.5 | (34.2%) |
| | N | 1 | | 33 | |
| Males | <50 pM | | | | |
| | 5^{th}-95^{th} | 1.2-11.7 | | 1.5-11.7 | |
| | Percentiles | | | | |
| | Mean (% CV) | 4.8 | (71.2%) | 6.9 | (48.2%) |
| | N | 33 | | 58 | |
| | 50-99 pM | | | | |
| | 5^{th}-95^{th} | 4.6-18.1 | | 3.6-15.8 | |
| | Percentiles | | | | |
| | Mean (% CV) | 9.4 | (49.1%) | 8.7 | (55.0%) |
| | N | 20 | | 39 | |
| | 100-149 pM | | | | |
| | 5^{th}-95^{th} | 7.2-14.0 | | 4.6-18.6 | |
| | Percentiles | | | | |
| | Mean (% CV) | 11.1 | (29.7%) | 11.3 | (50.5%) |
| | N | 4 | | 29 | |
| | ≧150 pM | | | | |
| | 5^{th}-95^{th} | 2.7-20.8 | | 6.0-18.0 | |
| | Percentiles | | | | |
| | Mean (% CV) | 10.7 | (75.0%) | 10.2 | (46.1%) |

| | | |
|---|---|---|
| n | 5 | 217 |

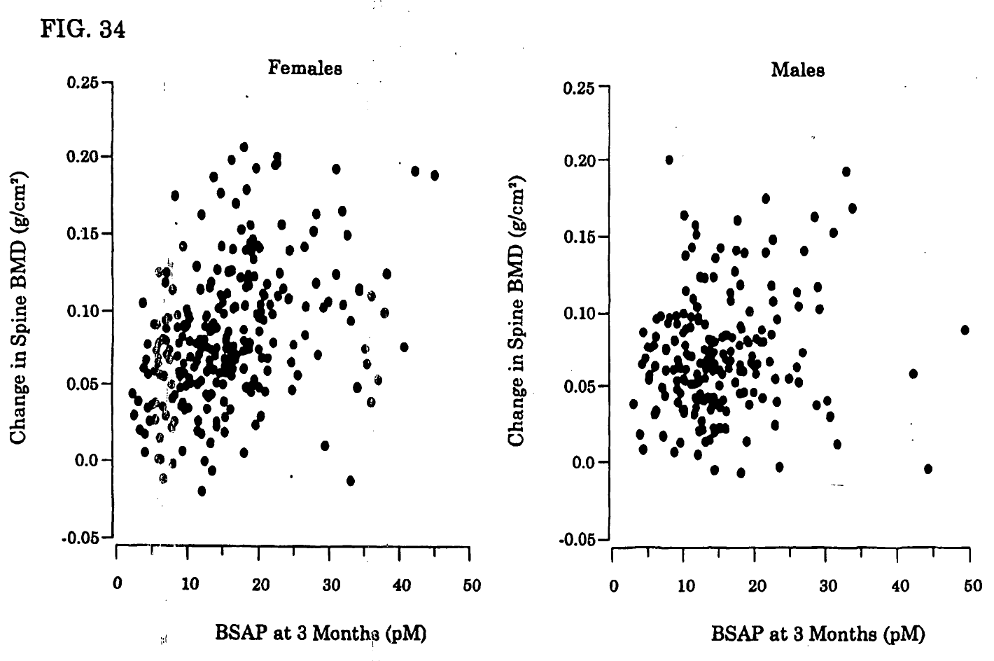

Bone-Specific Alkaline Phosphatase. High BSAP concentrations at 3 months are predictive of greater increase in total lumbar spine BMD at 12 months. This relationship seems to be more pronounced in postmenopausal women than in male patients. The relationship between BSAP concentration at 3 months and change in spine BMD is illustrated in FIG. 34 and Table 18 (below).

TABLE 18
Effect of BSAP on Change in Spine Bone Mineral Density

| | % Change in Spine BMD at 12 Months | | |
|---|---|---|---|
| BSAP at 3 Months | | Female Patients | Male Patients |

| <10 pM | | | | |
|---|---|---|---|---|
| 5^{th}-95^{th} | 0.5-15.3 | | 1.6-13.0 | |
| Percentiles | | | | |
| Mean (% CV) | 7.2 | (63.5%) | 7.1 | (64.9%) |
| n | 74 | | 45 | |
| 10-14.99 pM | | | | |
| 5^{th}-95^{th} | 1.5-18.9 | | 1.9-14.9 | |
| Percentiles | | | | |
| Mean (% CV) | 9.4 | (65.4%) | 7.9 | (59.5%) |
| N | 62 | | 78 | |
| 15-19.99 pM | | | | |
| 5^{th}-95^{th} | 3.4-24.7 | | 3.0-15.0 | |
| Percentiles | | | | |
| Mean (% CV) | 12.2 | (52.8%) | 8.3 | (46.2%) |
| N | 71 | | 45 | |
| ≧20 pM | | | | |
| 5^{th}-95^{th} | 4.6-20.9 | | 1.6-19.6 | |
| Percentiles | | | | |
| Mean (% CV) | 12.9 | (50.4%) | 10.0 | (59.6%) |

| | | |
|---|---|---|
| n | 65 | 40 |

Change in PICP and BSAP concentrations during LY333334 are correlated. BSAP concentrations at 3 months provide additional information, which is predictive of change in spine BMD for female patients. The indicator-response network shows that BSAP concentrations in male patients are not predictive of change in spine BMD, once the change in PICP concentration is taken into account.

Discussion

In view of the above correlations, the present invention provides a method for using change in a biochemical marker of bone formation for predicting subsequent change in spine bone mineral density resulting from repetitive administration of a parathyroid hormone to a human subject. In this method the biochemical marker of bone formation is an enzyme indicative of osteoblastic processes of bone formation or a product of collagen biosynthesis. This method comprises the steps of:
(a) determining the amount of difference for the subject between the level of the biochemical marker in a biological sample taken from the subject prior to administration of the hormone and the level in a sample taken after administration of hormone begins;
(b) comparing the amount of difference for the subject determined in step (a) with known amounts of difference for other human subjects determined as in step (a) to find a known amount of difference for other human subjects that is about the same as said that for the subject, wherein the parathyroid hormone has been administered to the other human subjects under the same conditions as for the subject of interest, and correlated amounts of subsequent change in spine bone mineral density resulting from administration of parathyroid hormone under these conditions are known for the known amounts of difference for other human subjects; and
(c) determining the known correlated amount of subsequent change in spine bone mineral density for the difference for the subject, thereby predicting that the subsequent change in spine bone mineral density (dBMD) due to administration of a parathyroid hormone to the subject will be that known correlated amount of subsequent change in spine bone mineral density.

In a preferred embodiment of this method, the repetitive administration is daily administration, the parathyroid hormone is hPTH(1-34), the biochemical marker of bone formation is the product of collagen biosynthesis in serum known as procollagen I C-terminal peptide (PICP) and the biological sample taken after administration of said hormone begins is taken about one month after administration of said hormone begins. This method may be used to predict change in spinal bone mineral density (dBMD) at a period of months or years, preferably about one year, after administration of the hormone begins. According to the invention, based on the correlations described in this Example, the method of predicting change in spine bone mineral density may further comprise a step in which the predicted dBMD determined in step (c) is adjusted for dose of PTH (e.g., 20 μg or 40 μg), for gender and age of the subjects, for base line PICP level of the subjects before administration of said hormone begins, and/or for a the concentration of bone-specific alkaline phosphatase determined at about 3 moths after administration of hormone begins. As one of ordinary skill would appreciate, such adjustments to the predicted dBMD determined in step (c) may be by reference to tables of correlated data (such as Tables 17 and 18, above), graphical displays of correlated date (such as FIGS. 28-31 herein). Such corrections also may be made using computer algorithms embodying correlations provided by the present invention.

The invention has been described with reference to various specific and preferred embodiments and techniques. However, it should be understood that many variations and modifications may be made while remaining within the spirit and scope of the invention. All publications and patent applications in this specification are indicative of the level of ordinary skill in the art to which this invention pertains.

1-46. (canceled)

47. A method for monitoring an effect of administration of a parathyroid hormone to a subject, comprising:
determining a level of an enzyme indicative of an osteoblastic process of bone formation, a product of collagen biosynthesis, a product of collagen degradation, or a combination thereof in a biological sample from the subject; and
correlating the level determined with an effect of administration of a parathyroid hormone.

48. The method of claim 47, wherein the enzyme indicative of an osteoblastic process of bone formation comprises a bone specific alkaline phosphatase.

49. The method of claim 48, further comprising:
determining an elevated level of the bone specific alkaline phosphatase in a period subsequent to initiation of administration of the parathyroid hormone to the subject;
correlating the elevated level of the bone specific alkaline phosphatase in the subject with a desired response to administration of the parathyroid hormone.

50. The method of claim 49, wherein the period subsequent to initiation of administration of the parathyroid hormone comprises a period of 0 to about 15 months after initiation of administration.

51. The method of claim 50, further comprising:
determining an elevated level of the procollagen I C-terminal propeptide in a period just after initiation of administration of the parathyroid hormone to the subject;
correlating the elevated level of the procollagen I C-terminal propeptide in the subject with a desired response to administration of the parathyroid hormone.

52. The method of claim 51, wherein the elevated level of procollagen I C-terminal propeptide correlates with the response of spinal bone mineral density to administration of the parathyroid hormone.

53. The method of claim 52, further comprising:
determining that the level of the procollagen I C-terminal propeptide has incerased to a peak level and subsequently declined to at or near control levels in the period subsequent to initiation of administration; and
correlating the increase to a peak level and subsequent decline with the effect of the subject undergoing a desired response to administration of the parathyroid hormone.

54. The method of claim 47, wherein the product of collagen degradation comprises an N-telopeptide.

55. The method of claim 54, further comprising:
determining that the level of N-telopeptide remains substantially constant in the period just after initiation of administration; and
correlating the substantially constant level with the effect on the subject undergoing a desired response to administration of the parathyroid hormone.

56. The method of claim 47, wherein the subject is a woman at risk of osteoporosis.

57. A kit for monitoring an effect of administration of a parathyroid hormone to a subject, comprising in a container a regent for determining a level of an enzyme indicative of an osteoblastic process of bone formation, a reagent for determining a level of a product of collagen biosynthesis, a reagent for determining a level of a product of collagent degradation, or a combination thereof; and instructions for said monitoring.

58. A method for using change in a biochemical marker of the formation for predicting subsequent change in spine bone mineral density resulting from repetitive administration of a parathyroid hormone to a human subject, wherein said biochemical marker of bone formation is a prouct of collgen biosynthesis, said method comprising the steps of:
(a) determining the difference for said subjet between the level of said biochemical marker in a biological sample taken from said subject prior to administration of said hormone and the level of said biochemical marker in a sample taken from said subject after administration of said hormone begins;
(b) comparing the difference for said subject determinied in step (a) with known differences fro other human subjects determined as in step (a) to find a known difference for other human subjects that is about the same as said amount of difference for said subject, wherein
said parathyroid hormone has been administered to said other human subjects under the same or similar conditions as for said subject, and
correlated amounts of subsequent change in spine bone mineral density resulting from administration of said parathryoid hormone under said same conditions are known for said known difference for other human subjects; and
(c) determining the known correlated amount of subsequent change in spine bone mineral density for said difference for said subject, thereby predicting that the subsequent change in spine bone mineral density due to said repetitive administration of a parathyroid hormone to said subject will be said known correlated amount of subsequent change in spine bone mineral density.

59. An article of manufacture comprising packaging material and a pharmaceutical composition contained within said packaging material, said composition comprising a parathyroid hormone consisting of amino acid sequence 1-34 of human parathyroid hormone and
said packaging material comprising printed matter which indicates that
said composition is effective for concurrently reducing the risk of both verebral and non-vertebral bone fracture in a male human subject at risk of or having osteoporosis when administered to said subject such that said parathyroid hormone is administered
without concurrent administration of an antiresorptive agent other than vitamin D or calcium,
in a daily dose of at least about 15 μg to about 40 μg for at least about 12 months to about 3 years.
